(19) United States
(12) Patent Application Publication
(10) Pub. No.: US 2025/0120852 A1
(43) Pub. Date: Apr. 17, 2025

(54) DEVICES FOR BLEEDING REDUCTION AND METHODS OF MAKING AND USING THE SAME

(71) Applicant: Bio 54, LLC, Chapel Hill, NC (US)

(72) Inventors: Michael Bruce WIGGIN, Raleigh, NC (US); Anton Travis MANASCO, Chapel Hill, NC (US); Peter Calvin COSTELLO, Raynham, MA (US); Kenneth Allen FOCHT, Needham, MA (US); Daniel P. SMITH, Portsmouth, RI (US); Michael W. METZ, Minneapolis, MN (US); John MURPHY, North Reading, MA (US)

(73) Assignee: Bio 54, LLC, Chapel Hill, NC (US)

(21) Appl. No.: 18/821,990

(22) Filed: Aug. 30, 2024

Related U.S. Application Data
(63) Continuation of application No. PCT/US2023/063606, filed on Mar. 2, 2023.
Application No. PCT/US2023/063606 is a continuation-in-part of application No. 17/728,894, filed on Apr. 25, 2022, now Pat. No. 11,622,893.
Application No. 17/728,894 is a division of application No. 17/522,736, filed on Nov. 9, 2021, now Pat. No. 11,654,057.
Application No. 17/522,736 is a continuation of application No. PCT/US2021/053641, filed on Oct. 5, 2021.
Application No. PCT/US2021/053641 is a continuation-in-part of application No. PCT/US2021/026714, filed on Apr. 9, 2021.
(60) Provisional application No. 63/315,915, filed on Mar. 2, 2022.
(60) Provisional application No. 63/087,532, filed on Oct. 5, 2020.
(60) Provisional application No. 63/090,768, filed on Oct. 13, 2020.
(60) Provisional application No. 63/007,543, filed on Apr. 9, 2020.

(51) Int. Cl. A61F 13/00; A61F 13/01; A61K 31/197

(52) U.S. Cl. CPC A61F 13/00085; A61F 13/00063; A61F 13/01042; A61K 31/197; A61F 2013/00285

(57) ABSTRACT

In some embodiments, a system includes an applicator pad, an application device (e.g., a grip), and a reservoir. The grip can be releasably couplable to the applicator pad and configured to dispose the applicator pad against a wound of a subject such that pressure can be transferred to the wound via the applicator pad to enhance hemostasis. The reservoir can be configured to contain medication to be released to the wound via the applicator pad.

### CROSS-REFERENCE TO RELATED APPLICATIONS

This application is a continuation of International Patent Application No. PCT/US2023/063606, entitled “Devices for Bleeding Reduction and Methods of Making and Using the Same,” filed Mar. 2, 2023, which claims priority to and the benefit of U.S. Provisional Patent Application Ser. No. 63/315,915, entitled “Devices for Bleeding Reduction and Methods of Making and Using the Same,” filed on Mar. 2, 2022, the disclosure of each of which is incorporated by reference herein in its entirety.

International Patent Application No. PCT/US2023/063606 is a continuation-in-part of U.S. patent application Ser. No. 17/728,894, entitled “Devices for Bleeding Reduction and Methods of Making and Using the Same,” filed on Apr. 25, 2022, which is a divisional of U.S. patent application Ser. No. 17/522,736, entitled “Devices for Bleeding Reduction and Methods of Making and Using the Same,” filed on Nov. 9, 2021, which is a continuation of International PCT Application No. PCT/US2021/053641, entitled “Devices for Bleeding Reduction and Methods of Making and Using the Same,” filed on Oct. 5, 2021, which is a continuation-in-part of International PCT Application No. PCT/US2021/026714, entitled “Devices for Bleeding Reduction and Methods of Making and Using the Same,” filed on Apr. 9, 2021, which claims priority to U.S. Provisional Patent Application Ser. No. 63/007,543, entitled “Devices for Bleeding Reduction and Methods of Making and Using the Same,” filed on Apr. 9, 2020, the disclosure of each of which is incorporated by reference herein in its entirety.

International PCT Application No. PCT/US2021/053641 also claims priority to U.S. Provisional Patent Application Ser. No. 63/087,532, entitled “Methods for Self-Treatment or Home Care Provider Treatment of Minor Wounds,” filed on Oct. 5, 2020, and U.S. Provisional Patent Application Ser. No. 63/090,768, entitled “Methods for Self-Treatment or Care Provider Treatment of Minor Wounds,” filed on Oct. 13, 2020, the disclosure of each of which is incorporated by reference herein in its entirety.

### BACKGROUND

Patients with minor injuries seek medical attention at primary care clinics, urgent care centers, and hospital emergency rooms. Many urgent care centers are not open twenty-four hours per day and seven days per week (24/7). Rural areas may have limited availability of these urgent care centers. A common type of visit to a hospital emergency room or urgent care center is a wound requiring cessation of bleeding, especially patients on anticoagulant or antiplatelet medications. As the population ages, more patients require these medications for a variety of conditions.

Better methods and devices are needed to allow patients, family members, and/or caretakers to safely treat at home, or at the place of the injury, minor wounds requiring the cessation of bleeding.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are devices, methods of using devices, and methods of making devices for treatment of bleeding, e.g. from minor wounds. In some embodiments, a system includes an applicator pad, an application device (e.g., a grip), and a reservoir. The grip can be releasably couplable to the applicator pad and configured to dispose the applicator pad against a wound of a subject such that pressure can be transferred to the wound via the applicator pad to enhance hemostasis. The reservoir can be configured to contain medication to be released to the wound via the applicator pad.

Additional features, aspects and/or advantages will be recognized and appreciated upon further review of a detailed description of the illustrative embodiments taken in conjunction with the accompanying drawings.

### BRIEF DESCRIPTION OF THE DRAWINGS

FIG. 1 is a cross-sectional view of a skin portion, showing the structures of the epidermis, derma, hypodermis, and subcutaneous layer.

FIG. 2 is a schematic illustration of a bleeding treatment system, according to an embodiment.

FIG. 3A is a schematic illustration of the bleeding treatment system of FIG. 3, with packaging removed from the bleeding treatment device, shown disposed near a wound on the skin of a patient.

FIG. 3B is a schematic illustration of the bleeding treatment device of FIG. 3A with an optional applicator seal removed, shown with a portion of the bleeding treatment device disposed adjacent to the wound on the skin of the patient.

FIG. 3C is a schematic illustration of the bleeding treatment device of FIG. 3A with the grip removed.

FIG. 3D is a schematic illustration of the bleeding treatment device of FIG. 3A, shown separated from the wound of the patient.

FIG. 4 is a flow chart of a method of using the bleeding treatment system of FIGS. 2 and 3A to 3D to treat a wound requiring cessation of bleeding.

FIG. 5 is a schematic illustration of a bleeding treatment system, according to an embodiment.

FIGS. 6A-6C are schematic illustrations of a bleeding treatment system, according to an embodiment.

FIGS. 7A-7D are schematic illustrations of a bleeding treatment system, according to an embodiment, in an initial configuration.

FIGS. 7E-7H are schematic illustrations of a bleeding treatment system in a wetting configuration.

FIG. 8 is a schematic illustration of a bleeding treatment system, according to an embodiment.

FIGS. 9A-9C are schematic illustrations of a bleeding treatment system, according to an embodiment.

FIGS. 10A-10C are schematic illustrations of a bleeding treatment system, according to an embodiment.

FIGS. 11A and 11B are schematic illustrations of a bleeding treatment system, according to an embodiment.

FIG. 12 is a schematic illustration of an applicator pad and a blood absorption pad, according to an embodiment.

FIG. 13 is a schematic illustration of a bleeding treatment system, according to an embodiment.

FIG. 14 is a flow chart of a method of using a bleeding treatment system, according to an embodiment.

FIGS. 15A-15E are schematic illustrations of a bleeding treatment system in various stages of use, according to an embodiment.

FIGS. 16A-16C are schematic illustrations of a bleeding treatment system in a wetting configuration, according to an embodiment.

FIGS. 16D-16F are schematic illustrations of the bleeding treatment system of FIGS. 16A-16C in a separated configuration.

FIGS. 17A and 17B are schematic illustrations of the bleeding treatment system of FIGS. 16A-16C, according to an embodiment.

FIG. 18 is a schematic illustration of a bleeding treatment system of FIGS. 17A-17B, according to an embodiment.

FIG. 19A is a schematic illustration of a bleeding treatment system, according to an embodiment.

FIG. 19B is a schematic illustration of a portion of the bleeding treatment system of FIG. 19A.

FIG. 19C is a schematic illustration of a portion of the bleeding treatment system of FIG. 19A, according to an embodiment.

FIG. 20 is a schematic illustration of a bleeding treatment system, according to an embodiment.

FIG. 21 is a schematic illustration of a bleeding treatment system, according to an embodiment.

FIGS. 22A and 22B are schematic illustrations of cross-sectional and top views of a bleeding treatment system, according to an embodiment.

FIGS. 23A-23C are schematic illustrations of a bleeding treatment system, according to an embodiment.

FIG. 24 is a schematic illustration of a bleeding treatment system, according to an embodiment.

FIGS. 25A-25C are schematic illustrations of a sequence of steps performed using a kit, according to an embodiment.

FIGS. 26A-26C are schematic illustrations of a sequence of steps performed using a kit, according to an embodiment.

FIG. 27 is a schematic illustration of a bleeding treatment system, according to an embodiment.

FIGS. 28A-28F are various views of a bleeding treatment system, according to an embodiment.

FIGS. 29A-29H are various views of a bleeding treatment system, according to an embodiment.

FIGS. 30A-30E are various views of a bleeding treatment system, according to an embodiment.

FIGS. 31A-31D are various views of a bleeding treatment system, according to an embodiment.

FIGS. 32A-32L are various views of a bleeding treatment system, according to an embodiment.

FIGS. 33A and 33B are views of an applicator pad of a bleeding treatment system, according to an embodiment.

FIG. 34 is an example of an applicator pad according to an embodiment.

FIGS. 35A-35D are various views of a bleeding treatment system, according to an embodiment.

FIG. 36A is an example of an applicator pad containing microneedles, according to an embodiment.

FIGS. 36B-36D are views of a region of an applicator pad in a vicinity of a microneedle, according to an embodiment.

FIG. 36E is a schematic illustration of another implementation of a microneedle, according to an embodiment.

FIG. 37 is a perspective view of an embodiment of a bleeding treatment system, according to an embodiment.

FIG. 38A is an exploded view of the embodiment of the bleeding treatment system, as shown in FIG. 37.

FIGS. 38B and 38C are perspective views of various components of the bleeding treatment system, according to the embodiment shown in FIG. 38A.

FIGS. 38D and 38E are top views of various components of the bleeding treatment system, according to the embodiment shown in FIG. 38A.

FIGS. 39 and 40 are cross-sectional views of the bleeding treatment system prior the use of the bleeding treatment system, according to the embodiment shown in FIG. 38A.

FIGS. 41 and 42 are cross-sectional views of the bleeding treatment system during the use of the bleeding treatment system, according to the embodiment shown in FIG. 38A.

FIGS. 43-45 show components of the bleeding treatment system, according to the embodiment shown in FIG. 38A.

FIGS. 46 and 47 are cross-sectional views of the bleeding treatment system after a drive plunger moves into reservoirs to urge medicine from the reservoirs, according to the embodiment shown in FIG. 38A.

FIGS. 48 and 49 are cross-sectional views of the bleeding treatment system after an applicator cap is removed from a main portion of the bleeding treatment system, according to the embodiment shown in FIG. 38A.

FIG. 50 show the applicator pad containing channels, according to the embodiment shown in FIG. 38A.

FIGS. 51-55 are various views of a bleeding treatment system, according to an embodiment.

FIGS. 56A-56J, 57A-B, 58A-58D, 59A-59D, 60A-60C, 61A-61C, 62A-62D, 63A-63F, 64A-64F, 65A-65E, and 66A-66F are various views of a bleeding treatment system, according to an embodiment.

### DETAILED DESCRIPTION

The detailed description herein serves to describe non-limiting embodiments or examples involving various inventive concepts and uses reference numbers for ease of understanding these examples. Common reference numbers between the figures refer to common features and structure having the same or similar functions, as will be understood. While various figures will have common reference numbers referring to such common features and structure, for purposes of conciseness, later figure descriptions will not necessarily repeat a discussion of these features and structure.

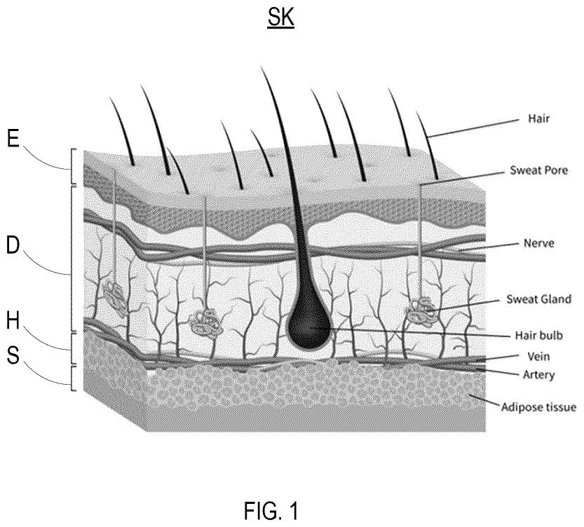

For reference, FIG. 1 illustrates a cross-sectional view of a skin portion SK of a human, e.g., of a subject to be treated. The skin portion SK includes an epidermis E, a derma D, a hypodermis H, and a subcutaneous layer S.

The systems and methods described herein can be configured to treat a wound and/or assist with cessation of bleeding of a wound that extends through the epidermal and/or dermal layer, is non-arterial, is not resulting in pulsatile bleeding, and has no subcutaneous fat or muscle exposed. In some embodiments, the systems and methods described herein can be self-administered (e.g., the administrator of the system and/or method is also the subject having the wound). In some embodiments, the systems and methods described herein can be administered to a subject having a wound by a person other than the subject, such as a caregiver (e.g., a non-medical home care provider such as a family member) or clinician. In some embodiments, the systems and methods described herein can be administered to a non-human patient such as a pet dog or a pet cat, and the method steps can be performed by a pet owner or veterinarian. In some embodiments, target wounds treatable by the systems and methods described herein may be less than 3 centimeters in length. In some embodiments, target wounds treatable by the systems and methods described herein in a home use setting may be wounds not requiring stitches or sutures to close. In some embodiments, target wounds treatable by the systems and methods described herein in a clinical setting may be wounds for which bleeding can first be attempted to be stopped using the systems and methods described herein before utilizing stitches or sutures if the systems and/or methods described herein are not effective after one use.

Potential patient populations that can be treated by the systems and methods described herein include any person with topical bleeds (i.e., bleeding from the skin). For example, target patient populations can include people with naturally-induced, drug-induced, or procedurally-induced increased susceptibility to bleeding and/or resistance to blood clotting. Naturally-induced susceptibility to bleeding and/or resistance to blood clotting can arise from a chronic condition such as hemophilia, Von Willebrand disease, vascular disorders (e.g., Osler-Weber-Rendu), coagulopathies, kidney failure, liver failure, bone marrow suppression (pathologically or medication-induced) platelet disorders, age (older people can have more friable skin), or conditions (e.g., a genetic condition) that render the person more accident prone or difficult to treat (including age and/or motor or cognitive deficits). Drugs that can induce susceptibility to bleeding and/or resistance to clotting can include: anticoagulation medications such as warfarin, heparin, factor Xa inhibitor, thrombin inhibitors, low-molecular weight heparin, dabigatran, argatorban, hirudin, rivaroxaban, apixaban, edoxaban, fondaparinux, enoxaparin, dalteparin, and bivalirudin; and antiplatelet medications such as platelet aggregation inhibitors such as aspirin (acetylsalicylic acid or ASA), cangrelor, ticagrelor, lopidogrel, prasugrel, cilostazol, clopidogrel, dipyridamole, ticlopidine, glycoprotein platelet inhibitors such as epifibatide, tirofiban, abciximab, and protease-activated receptor-1 antagonists such as vorapaxar. Anticoagulation medications can be taken for a wide variety of indications, such as deep venous thrombosis, pulmonary embolus, and atrial fibrillation. Medical procedures that can induce susceptibility to bleeding and/or resistance to clotting can include renal replacement therapy (e.g., hemodialysis), cardiopulmonary bypass, extra-corporeal membrane oxygenation (ECMO), chemical thrombolysis (with tissue plasminogen activating factor (TPA)), cardiac catheterization, peripheral vascular procedures (e.g., femoral-popliteal bypass, arterial thrombectomy), mechanical thrombectomy, angiography, and other neuro-interventional procedures and interventions. In some embodiments, any of the systems, methods, or devices herein can be used to treat melasma and/or sun spots. Although systems described herein are referred to as bleeding treatment systems and described with respect to treating wounds, any of the systems and devices described herein, such as the system 100 described below, can also be configured or used as a system or device for topically applying any therapeutic or other substance to any suitable type of target treatment area of a user that may benefit from application of the substance.

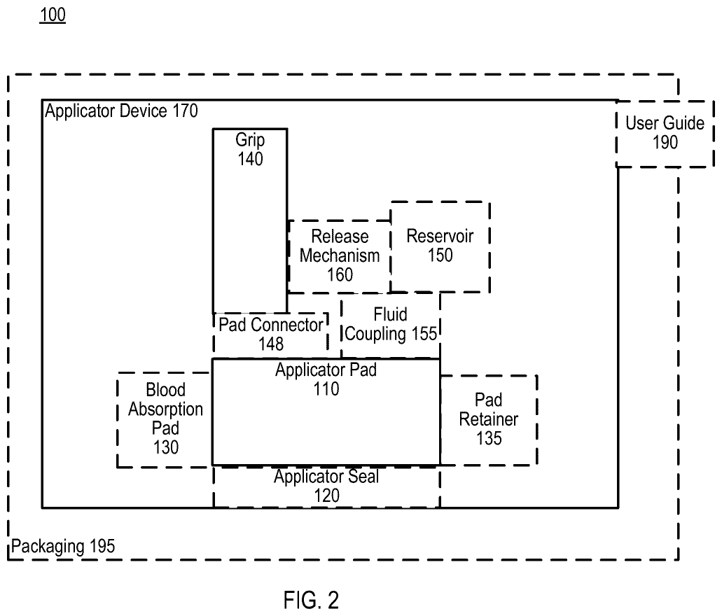

A bleeding treatment system 100 that can be used to treat a wound requiring cessation of bleeding is illustrated schematically in FIG. 2. As shown in FIG. 2, the bleeding treatment system 100 includes an applicator pad 110, an optional applicator seal 120 coupled to the applicator pad 110, an optional blood absorption pad 130 coupled to the applicator pad 110, one or more optional reservoirs 150 fluidically coupled to the applicator pad 110 via a fluid coupling 155 and containing one or more medications, an applicator device 170 releasably couplable to the applicator pad 110 and including a grip 140, an optional release mechanism 160 coupled to the reservoir 150, the fluid coupling 155, and the grip 140, an optional pad retainer 135 coupled to the applicator pad 110, an optional user guide 190 associated with the applicator device 170, and optional packaging 195 to contain the other components of the bleeding treatment system 100. The grip 140 can be releasably couplable to the applicator pad 110 via an optional pad connector 148.

The applicator pad 110 (also referred to as a topical applicator pad) is sized, configured, and formed of material, suitable for covering a wound treatable by the system 100. The applicator pad 110 may be formed of any one or more materials having suitable physical properties. For example, the material is preferably capable of absorbing blood. The material may also swell or expand upon absorbing fluid such as blood. The material may also be capable of containing, absorbing, wicking, or otherwise transporting one or more medications for application to a surface of the wound WD and/or the skin SK surrounding the wound. It should be biologically compatible with the skin SK and wound WD. Preferably the one or more materials are also non-reactive, or otherwise compatible with any medication(s) to be transported by the applicator pad 110, e.g., not alter the composition, delivery, or efficacy of the medication(s) or degrade or otherwise lose any of its desired physical properties upon exposure to the medication(s) over the maximum duration of the wound treatment. The material is preferably sufficiently stiff, resilient, etc. to be capable of applying a sufficient amount of pressure against the wound treatment to aid in achieving hemostasis, i.e., function as a hemostat. At least a bottom or distal surface of the applicator pad 110 (e.g., any surface exposed prior to use of the bleeding treatment system 100) can be covered or enclosed by the removable applicator seal 120 to prevent contamination of the applicator pad 110 and/or prevent undesired release of any medication(s) that may be contained in the applicator pad 110. The applicator pad 110 can be formed of a sponge material. The applicator seal 120 can be formed as a film or other thin layer of material non-reactive with the applicator pad 110. The applicator seal 120 can be removed from the applicator pad 110 prior to use (e.g., via peeling).

Suitable materials for the applicator pad 110 include polymers or other compositions, such as polyvinyl alcohol (PVA), polyurethane (hydrophilic or otherwise), polypropylene, which may be formed into foams (open or closed cell) (e.g., a porous expandable foam), natural fibers such as cotton, linen, wool, etc. in woven or non-woven (e.g. felt) form, and/or layered matrices of foam and/or gauze packing. The applicator pad 110 may be formed monolithically of a single material, or may be formed as a composite or other aggregation of different materials. For example, the applicator pad 110 may be primarily formed of one material, and have a relatively thin covering of a second material. The materials may achieve the desired functions in different ways. For example, absorption of blood may be achieved mechanically, e.g. by capillary wicking, and/or chemically, such as by absorption into, for example, molecular sieves or other desiccants, or combination with materials such as clays, e.g. kaolin, bentonite, montmorillonite, saponite, polygorskite, attapulgite, and/or sepiolite. In some embodiments, clay may be dispersed in a liquid medium. In some embodiments, the applicator pad 110 can include a hemostatic patch and a flexible insulating or wicking mechanism (also referred to as a backing portion or layer). In some embodiments, the hemostatic patch can include liquid medication, and the flexible insulating or wicking mechanism can isolate a hand applying pressure to the applicator pad 110 from the wound site and the liquid medication.

As explained in more detail below, the applicator pad 110 may include, incorporate, or embody a reservoir for medication(s), and a different material may be used to form or bound such a reservoir. In some embodiments, the applicator pad 110 can include a reservoir for dry or lipophilized medication that may be made flowable via fluid released from a reservoir of the applicator device 170 and/or blood from the wound. For example, the applicator pad 110 and/or a backing layer coupled to the applicator pad 110 can define a recessed or internal area within which powdered medication can be disposed in an initial configuration of the applicator pad 110. In some embodiments, the applicator pad 110 can define or include internal structures or passages to aid flow of medication through the applicator pad 110 and/or preferentially direct flow toward target wound tissue. In some embodiments, the applicator pad 110 can include a colored dye.

The bleeding treatment system 100 is preferably configured to deliver one or more medications to target wound tissue during treatment, e.g., via the applicator pad 110. The medication(s) may be contained in one or more reservoirs 150 (which, as noted above, may be separate from or incorporated into the applicator pad 110), may be selectively released from the reservoir(s) 150 by one or more release mechanisms 160, and may be conveyed from the reservoir(s) 150 to the applicator pad 110 by one or more fluid couplings 155. For example, the reservoir 150 may be implemented as a container (ampoule or the like) having a volume sufficient to contain a therapeutically effective amount of the medication and formed of material impermeable to, and non-reactive with, the medication or its constituents, such as glass, metal, plastic, polymer (e.g., a rigid polymer such as polyethylene, polypropylene, polyamide, polycarbonate), etc. In some embodiments, the reservoir 150 may be formed of a material that can be broken or punctured to release the contents of the reservoir 150 (e.g., plastic or glass). The reservoir 150 may have an opening through which the medication can be introduced into the reservoir 150 and/or selective selectively released therefrom. In some embodiments, the reservoir 150 can include a vent.

The grip portion 140 can be formed of any suitable material, with at least the distal end (e.g., the optional plate) being formed of a relatively rigid material capable of applying suitable pressure to the applicator pad 110. For example, the grip portion 140 (or at least a body of a first portion of the grip 140) can be formed of a rigid polymer such as polyethylene, polypropylene, polyamide, and/or polycarbonate.

The reservoir 150 may be contained, in whole or in part, within the applicator pad 110, or may be coupled thereto via a fluid coupling 155, such as a tube, wick, etc. The medication(s) may be selectively released from the reservoir 150 so that the medication(s) can be received in the applicator pad 110, e.g. by the release mechanism 160. The release mechanism 160 may be, for example a valve, which may be opened to establish fluidic communication between the reservoir 150 and the applicator pad 110, directly or via fluid coupling 155, and may also be selectively closed to fluidically isolate the medication(s) in the reservoir 150. The release mechanism 160 may be a removable or frangible cap or other closure closing an opening in reservoir 150. The reservoir 150 may itself be frangible, e.g., formed of glass that may be readily broken or at least partially of a material that bursts when the contents of the reservoir 150 are above a threshold pressure, and the medication(s) can be released from the reservoir 150 by causing or allowing the reservoir 150 to be broken. As noted above, in some embodiments the reservoir 150 may be a part of the applicator pad 110. For example, the material of the applicator pad 100 may be soaked or saturated with medication(s), and enclosed with the applicator seal 120 (e.g., a film or other thin layer of material impermeable to and non-reactive with the medication(s)). The medication(s) can be released from the reservoir 150 by removing the applicator seal 120, exposing the surface of the applicator pad 110 so that the medication(s) can be delivered to the wound tissue with which the surface of the applicator pad 110 is placed in contact.

In some embodiments, volume and material of the applicator pad 110 can be selected such that the amount of medication(s) contained in one or more reservoirs 150, whether separate from the applicator pad 110 or incorporated into the applicator pad 110, or the amount of medication(s) can be pre-soaked into the applicator pad 110 such that the applicator pad 110 is about 35% saturated (i.e., contains about 35% of the amount of the medication(s) that it is capable of containing). In some embodiments, the applicator pad 110 can be configured to be between about 67 and about 100% saturated by the amount of medication(s). In some embodiments, the applicator pad 110 is configured to be between about 25 and about 50% saturated by the amount of medication(s). In some embodiments, the applicator pad 110 can be pre-soaked or filled to hold a metered dose of medication. In some embodiments, the applicator pad 110 can be configured such that the volume of medication(s) is effective to treat the wound but such that the medication does not travel across the user's skin outside of the treatment area (e.g., down a user's arm or leg) (e.g., configured to be about 35% saturated or between about 25% and about 50% saturated).

The applicator pad 110 can be any suitable shape and/or size that is, preferably, sufficient to cover the target wound. For example, the perimeter of the applicator pad 110 can be formed as a circle, an oval, an ellipse, a square, a rounded square, a rectangle, a rounded rectangle, a triangle, a pentagon, a hexagon, or any other suitable shape. As described above, target wound sizes to be covered by the applicator pad 110 may be, for example, less than about 3 centimeters in length and/or width. In some embodiments, for example, the applicator pad 110 can have a square perimeter having side lengths of about 1.5 inches. In some embodiments, the applicator pad 110 can have a smaller area. In some embodiments, the applicator pad 110 can have a surface area between about 3.0 cm² and about 7.0 cm². In some embodiments, the applicator pad 110 can have a surface area between about 7.0 cm² and about 12.0 cm². In some embodiments, the applicator pad 110 can have a surface area of between about 2 cm² and about 25 cm². In some embodiments, the applicator pad 110 can have a surface area between about 3 cm² and about 10 cm² or between about 10 cm² and about 20 cm². For example, the surface area may be about 2 cm², about 3 cm², about 4 cm², about 5 cm², about 6 cm², about 7 cm², about 8 cm², about 9 cm², about 10 cm², about 11 cm², about 12 cm², about 13 cm², about 14 cm², about 15 cm², about 16 cm², about 17 cm², about 18 cm², about 19 cm², about 20 cm², about 21 cm², about 22 cm², about 23 cm², about 24 cm², or about 25 cm².

The applicator device 170 may be any suitable device manipulable by a user to dispose the applicator pad 110 into a desired location, e.g., adjacent a wound WD, and to deposit the applicator pad 110 in the desired location. For example, the applicator device 170 may be a mechanical syringe that includes a barrel having a distal end suitable for attachment to the applicator pad 110 and a proximal end that may be grasped by the user. In some embodiments, the reservoir 150, the release mechanism 160, and/or the fluid coupling 155 can be disposed within the barrel (e.g., between a plunger and the applicator pad 110). In some embodiments, the applicator device 170 can include a plunger movable relative to the barrel and to which a user can apply distally directed force to engage the distal end of the plunger with the reservoir 150 and to cause the contents of the reservoir 150 to be urged distally through the barrel, out of the distal end of the barrel, and into contact with the applicator pad 110. For example, the reservoir 150 can include a breakable neck portion that separates from a body portion of the reservoir 150 to release liquid from the reservoir 150 in response to the reservoir 150 being urged against an internal ramp or incline. As another example, a neck portion of the reservoir 150 can be broken from the reservoir due to engagement between a deformable sidewall of the applicator device 170 and the neck portion due to manipulation of the sidewall by a user.

In some embodiments, the reservoir 150 can include a weakened or pre-scored area (e.g., a circumferential region or sidewall region) configured to preferentially break when a breaking force is applied to the weakened or pre-scored area. For example, in some embodiments, the weakened or pre-scored area can be at a mid-point location of the reservoir 150 along a central axis of the reservoir 150, adjacent a shoulder of the reservoir 150, and/or can be on or near a first (e.g., a distal) or a second (e.g., a proximal) end of the reservoir 150. The reservoir 150 can be configured to break (e.g., at a weakened or pre-scored area) due to a rotational, bending, and/or orthogonal force (relative to or along the central axis of the reservoir 150) applied to the reservoir 150. In some embodiments, such a rotational, bending, and/or orthogonal force can be applied via twisting and/or bending the grip 140 within which the reservoir 150 is disposed. In some embodiments, the breaking force can be applied via displacing (e.g., squeezing) a portion of the grip (e.g., a deformable sidewall portion) into sufficiently forceful contact with a sidewall of the reservoir 150 to break the reservoir 150 and release the contents of the reservoir 150. In some embodiments, the breaking force can be applied via rotating or translating a second portion of the grip 140 relative to a first portion of the grip 140 to apply the breaking force to the reservoir 150.

In some embodiments, the release mechanism 160 can include a force concentrating component (also referred to as a stress concentrating component) (e.g., a ball such as a steel ball or projection such as a convex or sharp tipped projection like a carbide tip) disposed on an inner surface of the grip 140 and configured to be urged toward a sidewall of the reservoir 150 (e.g., as a result of bending of the grip 140, squeezing or pushing an exterior portion of the grip 140 toward the reservoir 150, or rotating a second portion of the grip 140 relative to a first portion of the grip 140) to concentrate a breaking force against a sidewall or end of the reservoir 150 and break (e.g., shatter) the reservoir 150. In some embodiments, the grip 140 can include a notched feature in a sidewall of the grip 140 such that the grip 140 can be more easily bent about a preferential axis or plane including the notched feature.

In some embodiments, the reservoir 150 and/or the grip 140 can be disposed in a vertical orientation during a wetting of the applicator pad 110 with the contents of the reservoir 150 and/or during application of pressure to the applicator pad 110 when disposed on a wound of the subject such that the reservoir 150 is elongated along a longitudinal axis intersecting a plane containing the applicator pad 110 in the initial configuration of the system 100. In some embodiments, the contents of the reservoir 150 can be configured to flow from the reservoir 150 to the applicator pad 110 through an open distal end of the grip 140. In some embodiments, the reservoir 150 and/or the grip 140 can be disposed in a horizontal orientation during a wetting of the applicator pad 110 with the contents of the reservoir 150 and/or during application of pressure to the applicator pad 110 when disposed on a wound of the subject such that the reservoir 150 is elongated along a longitudinal axis disposed substantially parallel to a plane containing the applicator pad 110 in the initial configuration of the system 100. In some embodiments, the contents of the reservoir 150 can be configured to flow from the reservoir 150 to the applicator pad 110 through a sidewall of the grip 140.

In some embodiments, the applicator device 170 can include a filter (not shown) between the reservoir 150 and the applicator pad 110 to prevent unwanted material (e.g., particles above a certain size and/or glass pieces) from reaching the applicator pad 110. In some embodiments, the applicator device 170 may not contain the reservoir 150, the release mechanism 160, and/or the fluid coupling 155, but instead just provide a handle by which the user may hold the applicator pad 110 to be able to apply the applicator pad 110 to the target wound tissue and apply distributed pressure to the applicator pad 110 to maintain pressure between the applicator pad 110 against the target wound tissue.

The grip 140 may be used to provide a distributed force (e.g., a uniformly distributed force) to the applicator pad 110, e.g., to increase the pressure that the applicator pad 110 may apply to target wound tissue during use, to enhance hemostasis. The grip 140 may be implemented in a variety of ways, including those described in specific embodiments below. For example, the grip 140 may include a plate (also referred to herein as a “fixation plate”) disposed on a distal end of and extending laterally from a handle portion of the grip 140 and configured to contact and apply a distributed force against the applicator pad 110. In some implementations, the plate can be shaped and sized such that the plate has a larger length and width than the target wound tissue such that the plate can urge a sufficiently large portion of the applicator pad 110 into contact with the target wound tissue and maintain uniformly distributed pressure between the applicator pad 110 and the target wound tissue for a period of time. Thus, the grip 140 can be used to provide a sufficient hemostatic force against the target wound tissue (e.g. to generate sufficient pressure given the stiffness or modulus of the material(s) of which the applicator pad 110 is formed). In some embodiments, the grip 140 or a portion of the grip may be deformable (e.g., squeezable) to urge fluid from the reservoir 150 disposed within the grip.

In some embodiments, the applicator device 170 can selectively release the applicator pad 110. For example, the grip 140 can releasably engage with the applicator pad 110 via the pad connector 148 to apply the applicator pad 110 to a surface of the patient (e.g., to the target wound tissue). The pad connector 148 can disengage from the applicator pad 110 such that the grip 140 and pad connector 148 can be separated from the applicator pad 110, leaving the applicator pad 110 on the surface of the patient. In some embodiments, the pad connector 148 can include one or more adhesive strips configured to couple the grip 140 to the applicator pad 110 and to be removed from the grip 140 and/or the applicator pad 110 when the applicator pad 110 is disposed in contact with the wound of the subject without displacing the applicator pad 110 relative to the wound such that the grip 140 can be separated from the applicator pad 110 without displacing the applicator pad 110 relative to the wound. In some embodiments, the pad connector 148 can include complementary hook-and-loop fastener portions coupled to the grip 140 and the applicator pad 110 such that the grip 140 can be releasably coupled to the applicator pad 110 via decoupling the complementary hook-and-loop fastener portions. In some embodiments, the pad connector 148 can include a first engagement feature coupled to the applicator pad 110 and a second complementary engagement feature coupled to the grip 140. The second complementary engagement feature can be configured to releasably engage with the first engagement feature (e.g., via receiving a portion of the first engagement feature within an opening of the second engagement feature due to, for example, relative rotation between the features, or via receiving a portion of the first engagement feature between opposing arms of the second engagement feature). In some embodiments, the pad connector 148 can include a latch portion disposed on the grip 140 configured to engage with a hook portion disposed on the applicator pad 110.

In some embodiments, the applicator pad 110 can include or be coupled to the pad retainer 135, which can be configured to maintain the applicator pad 110 in contact with the target wound tissue. The pad retainer 135 can include, for example, adhesive on a skin-contacting side of the applicator pad 110 (e.g., disposed around at least a portion of a perimeter of the applicator pad 110). In some embodiments, the pad retainer 135 can include an adhesive foam disposed about a perimeter of the applicator pad 110. In some embodiments, the pad retainer 135 can include adhesive strips having a first portion coupled to an upper surface of the applicator pad 110 and having a second portion configured to be coupled to a subject's skin to retain the applicator pad 110 in place relative to a wound on the skin. In some embodiments, the pad retainer 135 can include a bandage configured to be placed over at least a portion of the applicator pad 110. In some embodiments, the bandage can be formed as a wrap configured to be wrapped around a portion of the subject's body including the wound (e.g., an arm or a leg). In some embodiments, the bandage can be formed as a bandage having an adhesive perimeter greater than the perimeter of the applicator pad 110 and configured to be applied over the top of the applicator pad 110. In some embodiments, the bandage can include a convex portion configured to be aligned with the wound and placed in contact with the upper surface of the applicator pad 110 such that, when the applicator pad 110 is disposed on the wound and the bandage is applied over the applicator pad 110, the convex portion can apply targeted pressure to the wound via the applicator pad 110 that is greater than if the bandage did not include a convex portion. In some embodiments, after a period of time, the convex portion can be removed from the subject, leaving the applicator pad 110 in place in contact with the wound.

In some embodiments, the applicator device 170 can be implemented as a pipette. The pipette may be configured in a manner most suitable for the accuracy and precision needed for the size and location of the wound. For example, the pipette may be configured to be positioned within a patient's nostril.

The bleeding treatment system 100 may include a user guide 190. The user guide 190 may include instructions for operation of the bleeding treatment system 100 to treat a person experiencing a wound requiring cessation of bleeding. The instructions may be in the form of textual and/or graphical information, which may be presented on fixed substrate (e.g. paper) or on a display (e.g. screen), and/or may use other sensory modalities, including audible (spoken instructions) and/or tactile (haptic feedback to the user). The user guide 190 may be disposed on (e.g. printed on) or coupled to (e.g. mechanically attached) any one or more component(s) of the bleeding treatment system 100, including the applicator device 170, the packaging 190, the applicator pad 110, and/or the reservoir 150. The user guide 190 may be separate from any of the components of the bleeding treatment system 100, but may be associated therewith, e.g. disposed in the packaging 195 along with the other components of the bleeding treatment system 100. In some embodiments, the user guide 190 may be implemented in whole or in part in software usable on a device such as smart phone, e.g. may be the form of an “app” that can be downloaded onto the smart phone and launched by a user in preparation for using the bleeding treatment system 100.

In some embodiments, the user guide 190 or another informational label disposed on or included with the system 100 can include an information label including user instructions such as: “Only apply only to superficial bleeding areas less than 1 inch. Press down with the topical applicator pad over the wound for 5 minutes. Place an adhesive bandage over the wound. If bleeding does not stop, seek professional medical treatment. If you feel weak or dizzy, call 911 immediately and do not use this device. Not to be used in the mouth. Not to be used on deep wounds. Do not use if you have a history of seizures. Do not use for penetrating wounds or puncture wounds (gunshot, knife, etc.).”

In some embodiments, the user guide 190 or another informational label disposed on or included with the system 100 can include an information label including user instructions such as: “Only apply only to superficial bleeding wounds 3 inches or less in length. Push the release mechanism to break the medication. Allow the medication to fully cover the topical applicator. Press down with the device over the wound for 10 minutes. Place an adhesive bandage over the wound. If bleeding does not stop or if you feel dizzy, weak, or fatigued, seek professional medical treatment IMMEDIATELY. Not to be used in the mouth. Not to be used on deep wounds. Do not use if you have a history of seizures. Do not use for penetrating wounds or puncture wounds (gunshot, knife, etc.). Do not drink this medication. Keep away from children or infants.”

In some embodiments, the user guide 190 or another information label disposed on or included with the system 100 can include one or more statements indicating one or more of the following: (i) remove the applicator cap before pressing down with the device over the wound; and (ii) peel the topical applicator from the rest of the device and use an adhesive bandage to keep the topical applicator on the wound.

In some embodiments, the user guide 190 or another informational label or stamp on the system 100 can include an expiration date. For example, the system 100 may have a shelf life from a date of manufacture of three years, more than three years, or less than three years. In some embodiments, the user guide 190 or another informational label or stamp on the system 100 can include a lot number and/or a date of manufacture. In some embodiments, the user guide 190 or another informational label or stamp on the system 100 can include a bar code and/or Quick Response (QR) code.

The packaging 195 may be implemented in the same manner as any known medical device packaging, to contain the other components of the bleeding treatment system 100, to protect the components from the environment, and optionally to preserve sterility of the components. The packaging 195 is preferably configured to be readily opened by a user, e.g. by peeling a cover from a tray, when the user desires to access and use bleeding treatment system 100. The packaging 195 may be implemented in many other ways, including for example a bag or box. The system 100 is preferably intended for a single user and one wound site application. The system 100 is preferably intended to be discarded after use, and may optionally include a container for safe disposal. In some embodiments, the system 100 can be originally supplied to the user in the packaging 195 that can also be used for disposal. In some embodiments, the packaging 195 and/or disposal container can be a re-sealable bag and/or can include a biohazard label and/or disposal instructions.

The medication(s) described herein may be any medication that would be desirable to deliver to the patient experiencing a wound requiring cessation of bleeding, preparatory to or as part of treatment of the wound. Categories of medications may include vasoconstrictors, antifibrinolytics, antibiotics, recombinant clotting factor medications, local anesthetics, analgesics, buffering agents, calcium, alcohols/antiseptics (e.g., a chlorhexidine solution such as a solution of chlorhexidine and isopropyl alcohol and/or ethyl alcohol), or any combination thereof. A vasoconstrictor may be useful to help bleeding vessels constrict prior to or simultaneously with administration of a hemostatic medication such as an antifibrinolytic, and may desirably be delivered to the target wound tissue at or around the site of the bleeding before, during, and/or after application of applicator pad 110 to the target wound tissue. Suitable vasoconstrictors may include phenylephrine, oxymetazoline (Afrin), and epinephrine. An antifibrinolytic agent may be useful to prevent blood clot breakdown, and may also desirably be delivered to the wound tissue at or around the site of the bleeding before, during, and/or after application of applicator pad 110 to the target wound tissue. Suitable antifibrinolytics may include aminocaproic acid, tranexamic acid (TXA) (e.g., dry or lyophilized TXA or a liquid formulation including TXA), aprotinin, protaaminomethylbenzoic acid, and fibrinogen. Protamine, a reversal agent for the anticoagulant heparin could be used before, during, or after use of the above medications. The medication(s) can include, for example, protamine sulfate. As noted above, the bleeding treatment system 100 and medication(s) may be particularly helpful for treatment of patients who are susceptible to bleeding or for whom it may be difficult to achieve hemostasis, such as patients who are taking anticoagulation and/or antiplatelet medications (identified above).

In some embodiments, the medication can include a pharmaceutical composition including a therapeutically effective amount of TXA, one or more antibiotic(s), one or more anesthetic(s), one or more non-steroid anti-inflammatory drug(s), and/or an excipient or carrier that facilitates local administration. For example, in some embodiments, the therapeutically effective amount of tranexamic acid is between 1-70% by weight of the composition. In some embodiments, the one or more antibiotic(s) can include sulfacetamide, mupirocin, erythromycin, clindamycin, sulfadiazine, mafenide, tetracycline, bacitracin, neomycin, and polymyxin B. In some embodiments, the one or more antibiotic(s) can include bacitracin, neomycin, and polymyxin B. In some embodiments, the excipient or carrier permits the composition to remain in contact with a bleeding wound. In some embodiments, the excipient or carrier comprises an ointment, a cream, a liniment, a paste, a lotion, a gel, a hydrogel, a liposome, a spray, an aerosol, a solution, or an emulsion. In some embodiments, the excipient or carrier permits instillation of the composition. In some embodiments, the one or more anesthetic(s) can include lidocaine, proparacaine, procaine, tetracaine and combinations thereof. In some embodiments, the one or more non-steroid anti-inflammatory drug(s) can include ketorolac, ketoprofen, flurbiprofen, bromfenac, diclofenac and/or combinations thereof.

In some embodiments, the medication can include a TXA-containing composition. In some embodiments, the TXA-containing composition comprises about 200-300 mg/mL of TXA. In embodiments, the TXA-containing composition comprises about 200 mg/mL of TXA. In embodiments, the TXA-containing composition comprises about 300 mg/mL of TXA. In embodiments, the TXA-containing composition comprises about 250 mg/mL of TXA. In embodiments, the viscosity of the TXA-containing composition is from about 1 to 50 cps, wherein the viscosity is measured at 21° C. using the rotational viscosity Method III (Cone-and-Plate Rheometers) described in USP <912>. In embodiments, the viscosity of the TXA-containing composition is from about 1 to 100 cps, wherein the viscosity is measured at 21° C. using the rotational viscosity Method III (Cone-and-Plate Rheometers) described in USP <912>. In embodiments, the viscosity of the TXA-containing composition is from about 5 to 50 cps, wherein the viscosity is measured at 21° C. using the rotational viscosity Method III (Cone-and-Plate Rheometers) described in USP <912>. In some embodiments, the medication can include any of the compositions and/or a composition having any of the characteristics described in International Patent Application No. PCT/US2023/063500 to M. Powell et al., filed Mar. 1, 2023, entitled “Topical Tranexamic Acid Compositions and Methods of Use Thereof,” the disclosure of which is incorporated by reference herein in its entirety.

In some embodiments, the medication can include analgesics, including but not limited to, opiates such as codeine, morphine, oxycodone, etc.; acetaminophen; anti-inflammatory agents, including nonsteroidal anti-inflammatory drugs, aspirin, etc.; antibiotics or another antimicrobial drugs or compounds; antihistamines (e.g., cimetidine, chloropheniramine maleate, diphenhydramine hydrochloride, and promethazine hydrochloride); antifungal agents; ascorbic acid; rutin; thrombin; botanical agents; etc.; and combinations thereof. The medication can also include magnesium sulfate, sodium metaphosphate, calcium chloride, dextrin, and combinations thereof.

In some embodiments, the medication can include sterile water and/or normal saline (which can be included as a carrier). In some embodiments, the medication can include between about 50% and about 100% tranexamic acid and between about 50% and about 0% sterile water or normal saline. In some embodiments, the medication can include between about 10% and about 50% tranexamic acid and between about 90% and about 50% sterile water or normal saline. In some embodiments, the medication can include between about 50% and about 90% tranexamic acid and between about 10% and about 50% sterile water or normal saline. In some embodiments, the medication can include at least one of a liquid and a gel. In some embodiments, the medication can have a viscosity between about 0.75 millipascal-seconds and about 0.98 millipascal-seconds at about +25 degrees Celsius.

In some embodiments, the reservoir 150 can include an activation liquid such as sterile water or saline and the applicator pad 110 can include a dried (e.g., lyophilized) medication configured to be activated by the activation liquid upon release of the activation liquid from the reservoir 150. In some embodiments, the applicator pad 110 can include dried medication configured to be activated by blood flowing from the wound when the applicator pad 110 is disposed in contact with the wound such that activation liquid disposed within the applicator device 170 is not needed or is supplemental. In some embodiments, the applicator pad 110 can include wetting agents to promote dissolution of the dried medication.

In some embodiments, the medication(s) may comprise an antifibrinolytic in an amount of about 50 mg/mL to about 300 mg/mL, including about 50 mg/mL, about 100 mg/mL, about 150 mg/mL, about 200 mg/mL, about 250 mg/mL, or about 300 mg/mL. In some embodiments, the medication(s) may also include a colored dye to indicate that the medication(s) has been dispensed to the wound and is of a composition to be human viewable based on skin color and blood color (before and after clotting). In other embodiments, the medication(s) may not contain a colored dye.

In some embodiments, the amount of medication(s) (e.g., of an antifibrinolytic such as TXA) included in the reservoir and/or provided from the reservoir to the target wound (e.g., the therapeutically effective amount) can be between about 1 mg and about 20 mg, between about 1 mg and about 5 mg, between about 5 mg and about 10 mg, between about 10 mg and about 15 mg, between about 15 mg and about 20 mg, and/or between about 10 mg and about 20 mg. In some embodiments, a larger amount of medication(s) can be included for treatment of larger target wound sizes.

In some embodiments, the reservoir(s) 150 and/or release mechanism 160 are configured such that a metered dose can be provided from the reservoir (e.g., to the applicator pad 110 and/or to the patient). The metered dose (e.g., of IV TXA) can be the amount of liquid disposed within the reservoir(s) 150 prior to use of the system 100. In some embodiments, the metered dose can be between about 1.5 mL and about 2.5 mL, between about 2.5 mL and about 4.5 mL, between about 3 mL and about 10 mL, between about 3 mL and about 7 mL, between about 1 mL and about 20 mL, between about 1 mL and about 1.5 mL, between about 2.5 mL and about 5 mL, between about 5 mL and about 10 mL, between about 10 mL and about 15 mL, or between about 15 mL and about 20 mL. For example, the reservoir 150 can include a metered dose of about 1 mL, or about 2 mL, or about 3 mL, or about 4 mL, or about 5 mL, or about 6 mL, or about 7 mL, or about 8 mL, or about 9 mL, or about 10 mL, or about 11 mL, or about 12 mL, or about 13 mL, or about 14 mL, or about 15 mL, or about 16 mL, or about 17 mL, or about 18 mL, or about 19 mL, or about 20 mL.

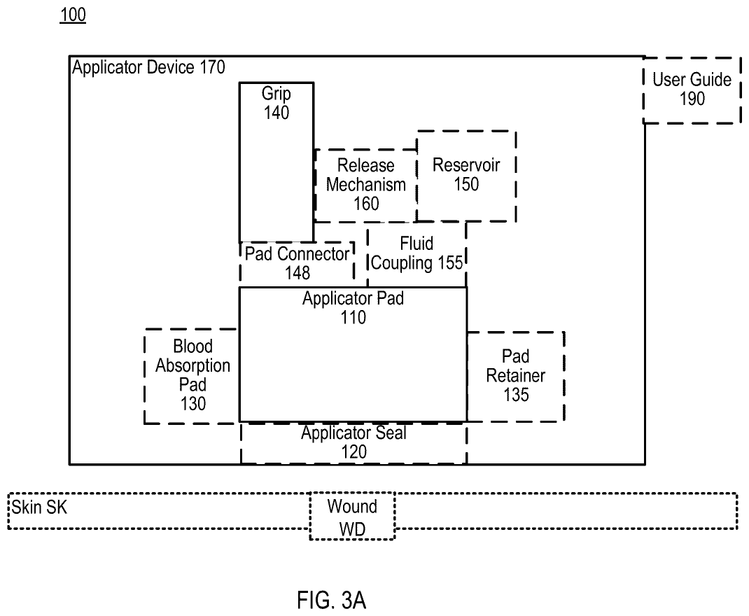

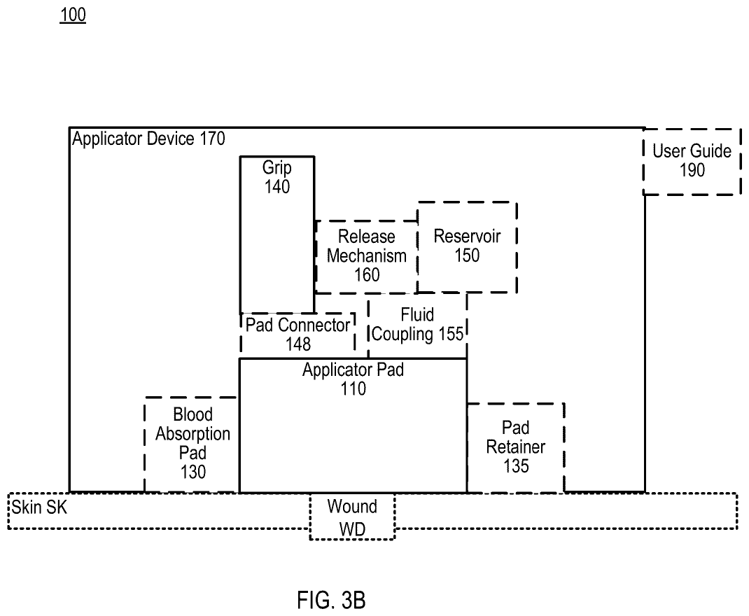

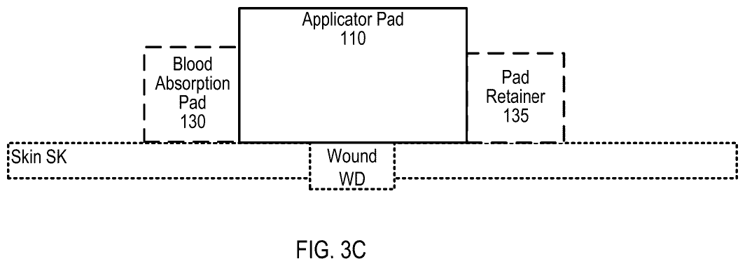

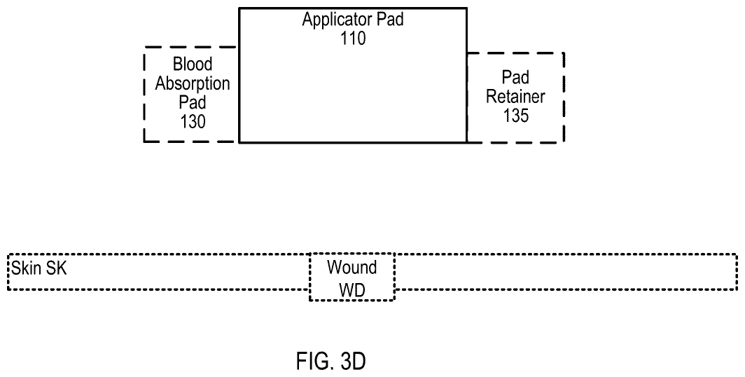

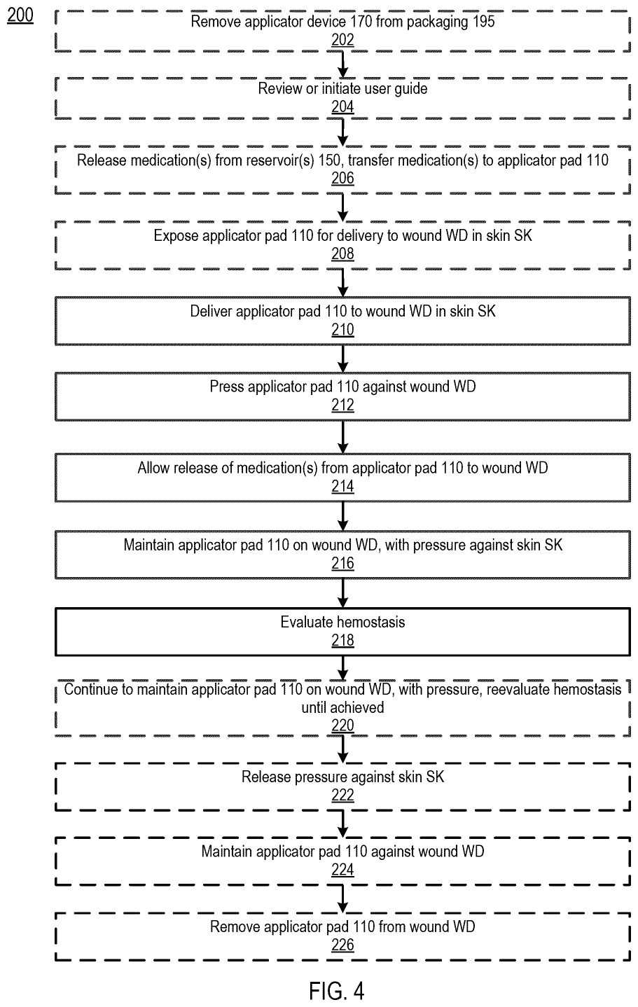

A method of using bleeding treatment system 100 to treat a wound requiring cessation of bleeding is shown in FIG. 4 and illustrated with reference to FIGS. 3A to 3D. In some embodiments, the bleeding treatment system 100 can be operated with one hand (e.g., the hand of the subject having the wound in need of treatment or the hand of a caregiver). As shown in FIG. 4 and illustrated in FIG. 3A, in some embodiments, the applicator device 170 can be removed, at 202, from the packaging 195. The user guide 190 can optionally be reviewed or initiated, at 204. At 206, optionally, medication(s) can be released from the reservoir(s) 150 and transferred to the applicator pad 110. The applicator pad 110 can be exposed, at 208, for delivery to a wound WD in skin SK (e.g., via removal of the optional applicator seal 120).

As shown in the flow chart of a method 200 shown in FIG. 4, and as illustrated in FIG. 3B, the applicator pad 110 can be delivered, at 210, to the wound WD in skin SK. As shown in FIG. 3A and referenced above, in some embodiments, the applicator pad 110 can be delivered to the wound WD in skin SK within, extending from, or coupled to the applicator device 170 such that the applicator pad 110 is in contact with the wound WD and an area of skin SK surrounding the wound WD.

The applicator pad 110 can be pressed, at 212, against the wound WD in the skin SK. For example, the applicator pad 110 can be pressed against the wound WD in the skin SK via applying pressure (e.g., pressure distributed across the wound WD) to the applicator pad 110 using the grip 140. Alternatively or additionally, in some embodiments, the applicator pad 110 can be pressed against the wound WD in the skin SK via the applicator pad 110 via applying pressure to the applicator pad 110 with fingers of the user.

Medication(s) can be allowed to be released, at 214, from the applicator pad 110 to the wound WD. For example, in some embodiments, medication(s) can be released from the reservoir 150 via interaction with the release mechanism 160 such that the medication(s) flow to the applicator pad 110 via the fluid coupling 155 and then from the applicator pad 110 to the wound WD. In some embodiments, as described above, the applicator pad 110 may include medication(s) prior to use of the system 100 (e.g., via being pre-soaked), such that removal of the applicator seal 120 and application of the applicator pad 110 to the wound WD (and/or pressure applied to the applicator pad 110 when in contact with the wound WD) allows or causes medication(s) to travel into contact with the wound WD. In some embodiments, rather than or in addition to releasing medication(s) from the reservoir 150, an activation liquid such as saline can be released from the reservoir 150 to activate medication(s) included in the applicator pad 110 (e.g., in an initial dry state) such that the combination of medication(s) from the applicator pad and the activation liquid can flow into contact with the wound WD. In some embodiments, blood from the wound WD can contact and activate medication(s) in the applicator pad 110 (e.g., in an initial dry state) such that the combination of medication(s) from the applicator pad and the blood can flow into contact with the wound WD.

The applicator pad 110 can be maintained, at 216, against the wound WD with pressure applied by the grip 140 against the wound WD. At 218, hemostasis can be evaluated. For example, a hemostasis condition of the wound WD can be evaluated to determine if the hemostasis condition meets a target hemostasis condition. The hemostasis condition of the wound WD can be evaluated via any suitable method. For example, after a preset period of time (e.g., five or ten to thirty minutes), the applicator pad 110 can be removed and the wound WD checked to determine whether or not hemostasis has been achieved.

In some embodiments, after evaluating hemostasis at 218, the applicator pad 110 can continue to be maintained, at 220, against the wound WD while continuing to apply pressure to the wound WD and hemostasis can be reevaluated until achieved. For example, if the hemostasis condition of the wound WD fails to meet a target hemostasis condition, the applicator pad 110 can be maintained against the wound WD applying pressure to the wound WD for a period of time. The hemostasis condition can then be reevaluated to determine if the target hemostasis condition has been met. Such a cycle can continue until the target hemostasis condition has been met, at which time the applicator pad 110 can be removed from the wound WD.

In some embodiments, as illustrated in FIG. 3C, prior to removal of the applicator pad 110 from the wound WD, pressure can be released, at 222, against the wound WD. For example, in the instance of a grip 140 being used to apply pressure to the applicator pad 110, the grip 140 can be disengaged from the applicator pad 110 to reduce the pressure of the applicator pad 110 against the wound WD. In some embodiments, the applicator pad 110 can be maintained against the wound WD, at 224, after the pressure is released (e.g., after removal of the grip 140). In some embodiments, the applicator pad 110 can be maintained in place against the wound WD by the pad retainer 135. In some embodiments, a bandage or wrap can be applied to the applicator pad 110 to maintain the applicator pad 110 against the wound WD for a period of time. In some embodiments, the applicator pad 110 can continue to apply pressure to the wound WD after removal of the grip 140 (e.g., due to remaining in contact with the wound WD). In some embodiments, the applicator pad 110 can continue to apply pressure to the wound WD at least in part due to swelling of the applicator pad 110 or the blood absorption pad 130 due to liquid (e.g., blood) absorption. As illustrated in FIG. 3D, the applicator pad 110 can be removed, at 226, from the wound WD.

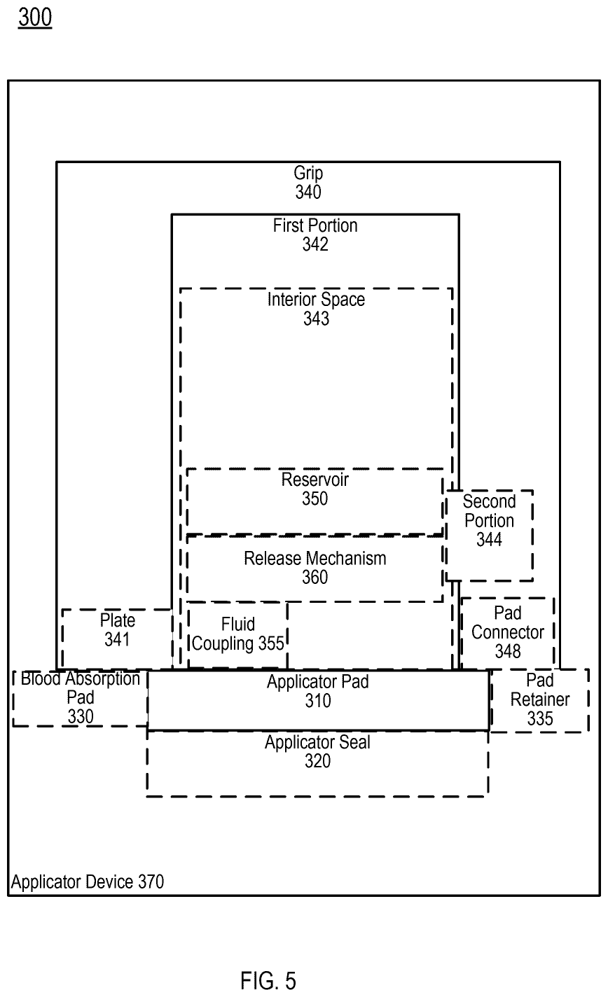

FIG. 5 is a schematic illustration of a bleeding treatment system 300. The system 300 can be the same or similar in structure and/or function to any of the bleeding treatment systems described herein. The system 300 includes an applicator device 370, an applicator pad 310, and an optional reservoir 350. The applicator device 370 includes a grip 340 having a first portion 342 (also referred to as a “first section”). The first portion 342 optionally defines an interior space 343. In some embodiments, the first portion 342 optionally defines an open distal end such that the interior space 373 can be fluidically coupled to the applicator pad through the distal end of the first portion 342.

The first portion 342 can be formed in any suitable shape. For example, the first portion 342 can include a cylindrical or tubular housing. In some embodiments, the first portion 342 can include or be coupled to a plate 341 disposed at the distal end of the first portion 342 and configured to apply pressure to the applicator pad 310 and thus a target wound. The plate 341 may form a bottom surface of the grip 340. In some embodiments, such as when the grip 340 does not include a reservoir, the plate 341 can be formed as a continuous plate. In some embodiments, the plate 341 may be formed as a flange (e.g., a circumferential flange) extending from a bottom edge of the first portion 342.

In some embodiments, the grip 340 includes an applicator seal 320 configured to cover at least the distal or skin-contacting surface of the applicator pad 310 to prevent contamination of the applicator pad 310 and, optionally, leakage from or drying of the applicator pad 310 prior to use. The applicator seal 320 can be formed, for example, as a film (e.g., a peelable film). In some embodiments, the applicator seal 320 can be formed of a shrink wrap material covering the distal end of the applicator pad 310 and positioned over at least a portion of the length of the first portion 342.

The applicator pad 310 can be the same or similar in structure and/or function to any of the applicator pads described herein. For example, the applicator pad 310 can include and/or be coupled to an optional blood absorption pad 330 and/or an optional pad retainer 335. The blood absorption pad 330 and the pad retainer 335 can be the same or similar in structure and/or function to any of the blood absorption pads or pad retainers, respectively, described herein.

As shown in FIG. 5, the reservoir 350 can be disposed in the optional interior space 343. In some embodiments, the reservoir 350 can be configured to be displaced and/or pressurized such that liquid (e.g., medication and/or activating liquid such as saline) within the reservoir 350 flows from the reservoir 350 to the applicator pad 310. For example, the reservoir 350 can optionally include a release mechanism 360 and/or a fluid coupling 355 that may be the same or similar in structure and/or function to any of the release mechanisms and/or fluid couplings described herein. In some embodiments, the fluid coupling 355 can be a portion of the interior space 343 defined by the housing of the first portion 342 via which liquid can flow from the reservoir 350 to the applicator pad 310. In some embodiments, the fluid coupling 355 includes a separate tubular member defining a lumen through which liquid can flow from the reservoir 350 to the applicator pad 310.

In some embodiments, the delivery device 370 can include an optional second portion 344 (also referred to as a “second section”). In some embodiments, the second portion 344 can be configured to engage with the reservoir 350 and/or the release mechanism 360 to initiate transfer of liquid from the reservoir 350 to the applicator pad 310. In some embodiments, the second portion 344 can be formed as a plunger and advanced by a user (e.g., a self-administering home user, a caregiver, or a patient) relative to the first portion 342 (e.g., at least partially into the interior space 343 or while in contact with the reservoir 350 to advance the reservoir 350 itself) to apply pressure to the reservoir 350. In some embodiments, the second portion 344 can be disposed on a sidewall of the first portion 342 and configured to be urged laterally into the interior space 343 to apply pressure to the reservoir 350.

In some embodiments, the interior space 343 can be at least partially defined by an inner surface of a sidewall of the grip 340, and can be defined by the first portion 342 and/or the second portion 344. In some embodiments, the inner surface and/or the outer surface of the sidewall of the grip 340 (including the first portion 342 and the second portion 344) can have any suitable shape, such as a circular cylindrical shape, an oval cylindrical shape, an elliptical cylindrical shape, a tubular shape having two opposing flat sides coupled together by two opposing curved sides, and/or a prism shape, including a cube, rounded square prism, rectangular prism, rounded rectangular prism, triangular prism, pentagonal prism, hexagonal prism, etc. In some embodiments, the inner surface of the grip 340 defining the interior space 343 includes one or more rounded or beveled edges. In some embodiments, the inner surface of the grip 340 defining the interior space 343 includes a rounded rectangular prism shape with one or more rounded edges. In some embodiments, the interior space 343 is cylindrical.

In some embodiments, the release mechanism 360 can be coupled to the reservoir 350 and configured to transition from a closed condition to an open condition to allow medication to flow from the reservoir 350 and to the applicator pad 310 via the fluid coupling 355 in response to a pressure above a threshold pressure within the reservoir 350 (e.g., applied by the distal end of the second portion 344). For example, in some embodiments, the release mechanism 360 can include a weakened or frangible portion of a sidewall of the reservoir 350. In some embodiments, the release mechanism 360 includes a valve that transitions from a closed to an open condition upon an internal pressure of the reservoir 350 rising above a threshold pressure. In some embodiments, the release mechanism 360 can be a frangible portion of the reservoir 350 configured to break when pushed against an internal obstruction (not shown) such as a ramp portion within the interior space 343 such that the medication within the reservoir 350 can be released from the reservoir 350. In some embodiments, the first portion 342 can include a filter (not shown) between the reservoir 350 and the applicator pad 310 to prevent unwanted material from reaching the applicator pad 310.

Prior to use, the applicator device 370 can be disposed in an initial configuration. In the initial configuration, the applicator pad 310 can be coupled to the distal end of the grip 340. The applicator seal 320 can cover the applicator pad 310. To use the applicator device 370, the applicator seal 320 can be optionally removed and the grip 340 can be used to apply the applicator pad 310 to a wound. In embodiments including the reservoir 350, the release mechanism 360, and the fluid coupling 355, liquid (e.g., medication and/or activating agent such as saline) can be contained within the reservoir 350 prior to and optionally after applying the applicator pad 310 to the wound. To release the liquid from the reservoir 350 such that the liquid flows to the applicator pad 310, the second portion 344 can be configured to be moved relative to the first portion 342 (e.g., via translation relative to the first portion 342, radial movement relative to the first portion 342, and/or rotational movement relative to the first portion 342) to engage with the reservoir 350 and/or the release mechanism 360.

The applicator pad 310 can optionally be pressed against target wound tissue using the grip 340. The applicator pad 310 can optionally be maintained relative to the wound (e.g., without maintaining the grip 340 against the applicator pad 310) via coupling the applicator pad 310 to the skin via the pad retainer 335. The applicator pad 310 can be optionally separated from the applicator pad 310 via releasing the applicator pad 310 from the pad connector 348.

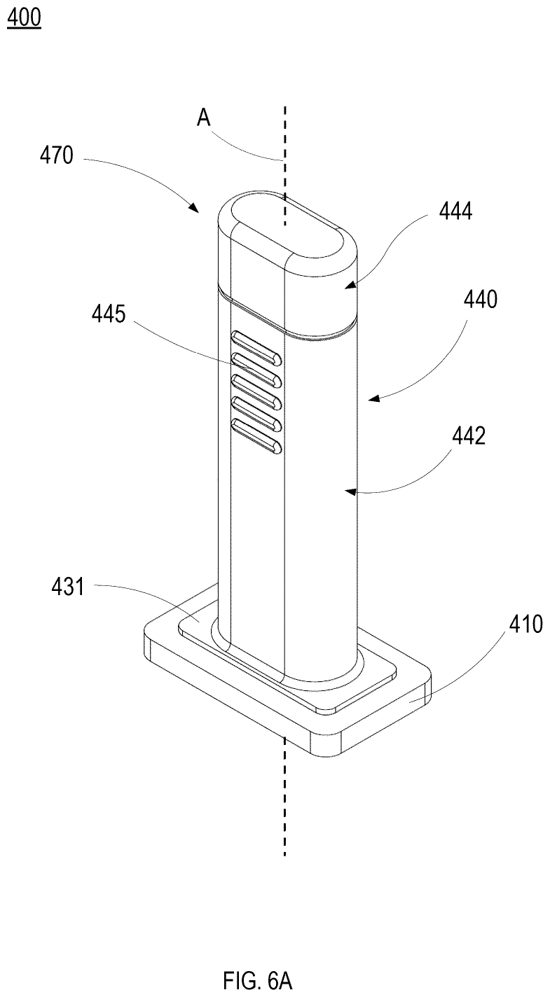

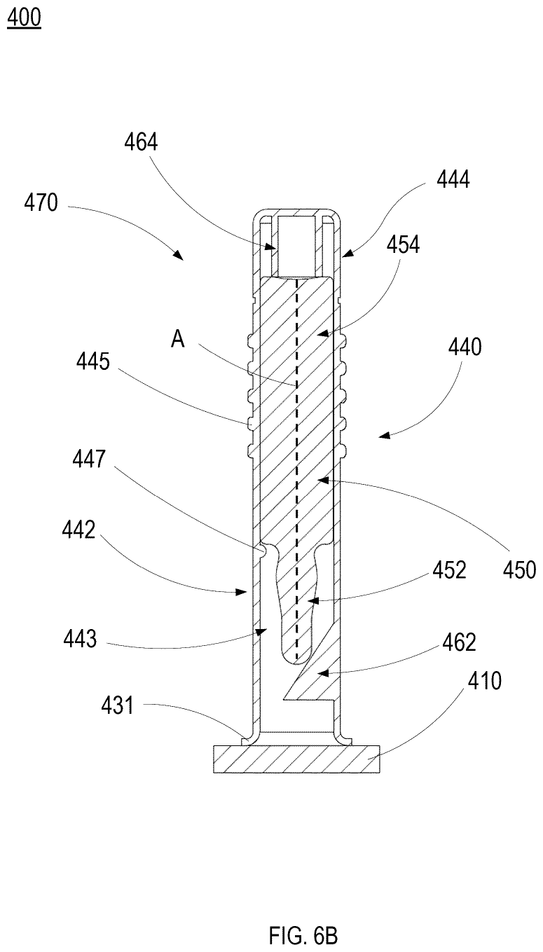

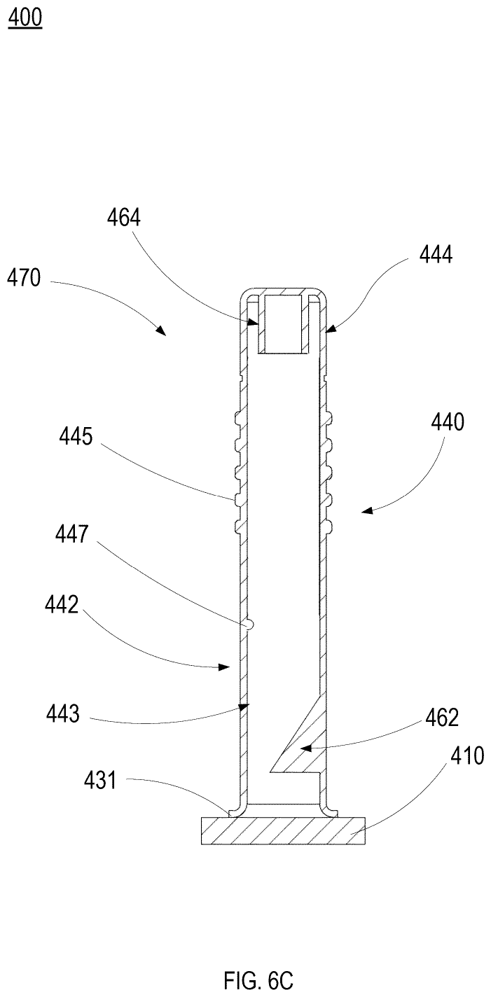

FIGS. 6A and 6B are a perspective view and a cross-sectional view of a bleeding treatment system 400. FIG. 6C is a cross-sectional view of the bleeding treatment system with a reservoir 450 of the bleeding treatment system 400 not shown. The system 400 can be the same or similar in structure and/or function to any of the delivery or treatment systems described herein. For example, the system 400 includes an applicator device 470, a reservoir 450, and an applicator pad 410. The applicator device 470 includes a grip 440 defining an interior space 443 and having an open distal end, a closed proximal end, and extending along a central longitudinal axis A. The grip 440 has a first portion 442 and a second portion 444, the second portion 444 being movable relative to the first portion 442. The reservoir 450 can be formed as an ampoule having a neck portion 452 and a body portion 454. The neck portion 452 can be narrower than the body portion 454 (e.g., can have a smaller diameter). The reservoir 450 can be disposed within the interior space 443 of the grip 440. The reservoir 450 can be oriented such that the neck portion 452 is distal of the body portion 454 and therefore closer to the open end of the grip 440. The interior space 443 can be at least partially defined by an inner surface of a sidewall of the grip 440, and can be defined by the first portion 442 and/or the second portion 444. The grip 440 (e.g., the inner surface and/or the outer surface) can be formed as shown in FIG. 6A, having a tubular shape having two opposing flat sides coupled together by two opposing curved sides. In some embodiments, the grip 440 can have any suitable shape, such as is described above with respect to the grip 340.

The first portion 442 can include a plate 431 having a distal or bottom surface extending laterally relative to the central axis A of the grip 440. The applicator pad 410 can be disposed on a distal end of the first portion 442 (e.g., coupled to the distal surface of the plate 431). The first portion 442 can include a ramp portion 462 (also referred to as a first release mechanism portion) extending partially into the interior space 443 of the first portion 442 such that the neck portion 452 can contact the ramp portion 462. The first portion 442 can also include one or more retaining portions 447 (e.g., flexible tab or bump portions) extending toward the central axis A of the first portion 442 and configured to contact a shoulder of the body portion 454 of the reservoir 450 as shown in FIG. 6B to retain the reservoir 450 in an initial proximal position until sufficient force is applied to a proximal end of the reservoir 450 to advance the reservoir 450 relative to the one or more retaining portions 447. The first portion 442 can also include one or more grip features 445 on an exterior surface of a sidewall of the first portion 442 to improve the grip of the user during handling of the grip 440. The grip features 445 can include any suitable surface feature to increase grip or friction, such as one or more ridges (as shown in FIG. 6A), concave and/or convex curvatures, and/or a textured or course surface.

The second portion 444 can include a projection 464 (also referred to as a second release mechanism portion). The first portion 442 and the second portion 444 can be coupled such that at least a portion of the second portion 444 can be advanced relative to the first portion 442 towards a distal end of the first portion 442. For example, the first portion 442 can be coupled to the second portion 444 via any suitable coupling mechanism, such as, for example, a flexible circumferential portion. To release liquid within the reservoir 450 such that the liquid flows to the applicator pad 410, the second portion 444 can be advanced distally relative to the first portion 442 (e.g., via pressing on a proximal end of the second portion 444) such that the second release mechanism portion 464 applies a force to the proximal end of the reservoir 450 to urge the reservoir 450 distally such that contact between the neck portion 452 of the reservoir 450 and the ramp 462 causes the neck portion 452 to separate (e.g., break) from a remainder of the reservoir 450 and allow liquid to flow from the reservoir 450, out of the distal end of the first portion 442, and to the applicator pad 410, resulting in the applicator pad 410 being wetted with the liquid from the reservoir 450.

In some embodiments, the first portion 442 can include a filter to prevent unwanted material (e.g., particles above a certain size and/or glass pieces) from reaching the applicator pad 410. Before or after wetting the applicator pad 410 with the liquid, the distal surface of the applicator pad 410 can be disposed against a wound of the subject. The grip 440 can then be maintained against the applicator pad 410 such that the plate 431 applies distributed pressure to the applicator pad 410 for a period time, as described with respect to the method 200 above. In some embodiments, the first portion 442 and/or the second portion 444 can be deformable (e.g., squeezable) to urge fluid (e.g., medication) from the reservoir 450. In some embodiments, the bleeding treatment system 400 can be operated with one hand (e.g., the hand of the subject having the wound in need of treatment). For example, the user can place a thumb on the proximal end of the second portion 444 and wrap the remaining fingers of the same hand around the sidewall of the grip 440 (e.g., around the first portion 442), with at least some of the remaining fingers in contact with the grip features 445. While gripping the grip 440 in this configuration, either before or after pressing the applicator pad 410 against the wound, the user can press the second portion 444 toward the applicator pad 410 with the user's thumb to release liquid from the reservoir 450 as described above.

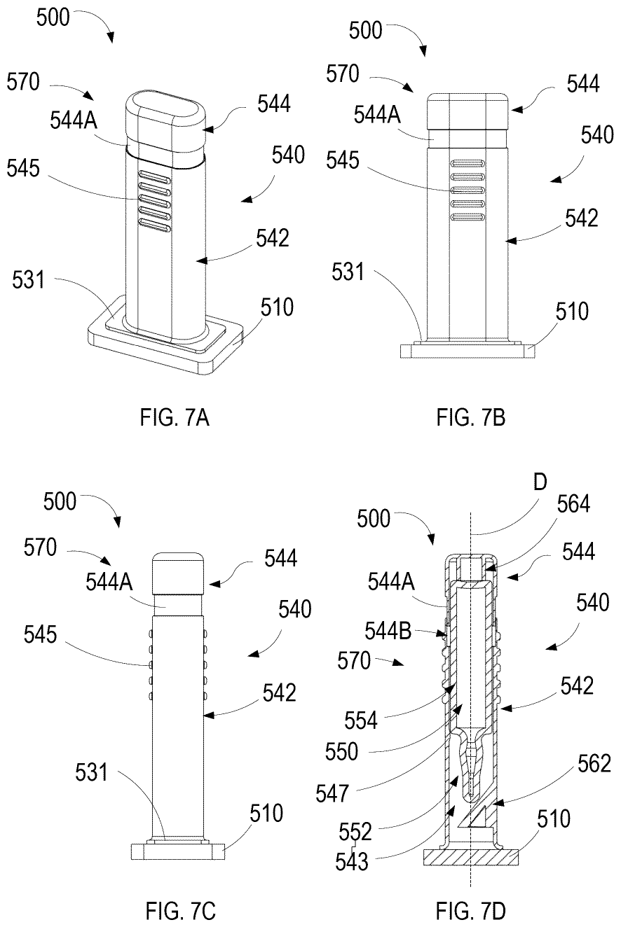

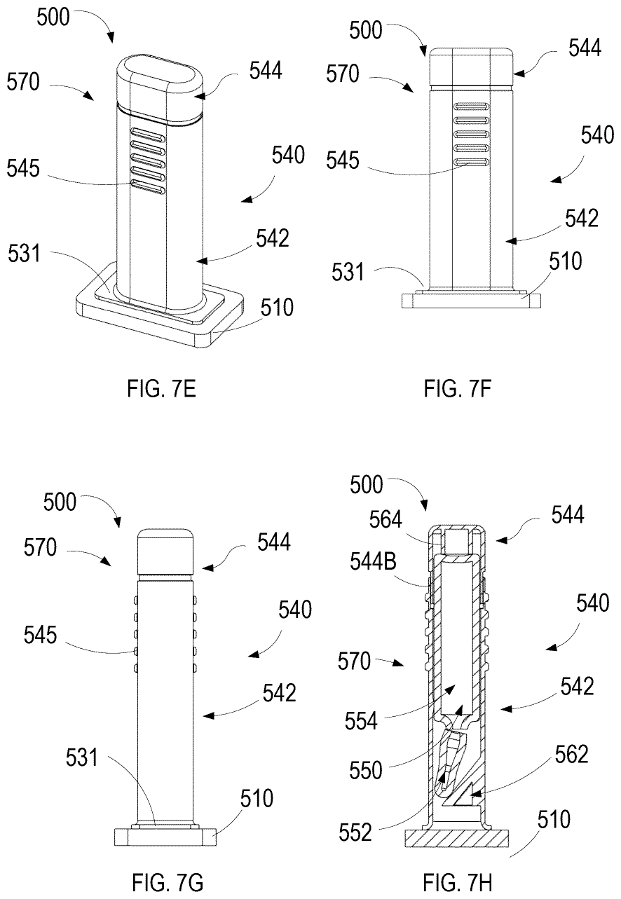

FIGS. 7A-7D are schematic illustrations of a perspective, front, side, and cross-sectional view, respectively, of a bleeding treatment system 500 in an initial configuration. FIGS. 7E-7H are schematic illustrations of a perspective, front, side, and cross-sectional view, respectively, of the bleeding treatment system 500 in wetting configuration. The system 500 can be the same or similar in structure and/or function to any of the delivery or treatment systems described herein, such as, for example, the bleeding treatment system 400. For example, the system 500 includes an applicator device 570, a reservoir 550, and an applicator pad 510. The applicator device 570 includes a grip 540 defining an interior space 543 and having an open distal end, a closed proximal end, and extending along a central longitudinal axis D. The grip 540 has a first portion 542 and a second portion 544, the second portion 544 being movable relative to the first portion 542. The reservoir 550 can be formed as an ampoule having a neck portion 552 and a body portion 554. The neck portion 552 can be narrower than the body portion 554 (e.g., can have a smaller diameter). The reservoir 550 can be disposed within the interior space 543 of the grip 540. The reservoir 550 can be oriented such that the neck portion 552 is distal of the body portion 554 and therefore closer to the open end of the grip 540. The interior space 543 can be partially defined by an inner surface of a sidewall of the first portion 542 and the second portion 544.

The first portion 542 can include a plate 531 having a distal or bottom surface extending laterally relative to the central axis D of the grip 540. The applicator pad 510 can be disposed on a distal end of the first portion 542 (e.g., coupled to the distal surface of the plate 531). The first portion 542 can include a ramp portion 562 (also referred to as a first release mechanism portion) extending partially into the interior space 543 of the first portion 542 such that the neck portion 552 can contact the ramp portion 562. The first portion 542 can also include one or more retaining portions 547 (e.g., flexible tab or bump portions) extending toward the central axis A of the first portion 542 and configured to contact a shoulder of the body portion 554 of the reservoir 550 as shown in FIG. 6B to retain the reservoir 550 in an initial proximal position until sufficient force is applied to a proximal end of the reservoir 550 to advance the reservoir 550 relative to the one or more retaining portions 547. The first portion 542 can also include one or more grip features 545 on an exterior surface of a sidewall of the first portion 542 to improve the grip of the user during handling of the grip 540.

The second portion 544 can include a projection 564 (also referred to as a second release mechanism portion) extending into the interior space 543. The first portion 542 and the second portion 544 can be coupled such that at least a portion of the second portion 544 can be advanced relative to the first portion 542 towards a distal end of the first portion 542. For example, the second portion 544 can include a slidable engagement portion 544A and the first portion 542 can include a receiving portion 544B having a corresponding shape and a larger perimeter than the outer diameter of the slidable engagement portion 544A such that the slidable engagement portion 544A can be translated into the receiving portion 544B (e.g., via pressing on a proximal end of the second portion 544 and/or gripping opposing sidewalls of the second portion 544 and advancing the second portion 544 distally). During the translation of the slidable engagement portion 544A into the receiving portion 544B, the second release mechanism portion 564 can apply a force to the proximal end of the reservoir 550 to urge the reservoir 550 distally such that contact between the neck portion 552 of the reservoir 550 and the ramp 562 causes the neck portion 552 to separate (e.g., break) from a remainder of the reservoir 550 and allow liquid to flow from the reservoir 550, out of the distal end of the first portion 542, and to the applicator pad 510, resulting in the applicator pad 510 being wetted with the liquid from the reservoir 550.

In some embodiments, the first portion 542 can include a filter (e.g., disposed between the ramp 562 and the distal opening of the grip 540) to prevent unwanted material (e.g., particles above a certain size and/or glass pieces) from reaching the applicator pad 510. Before or after wetting the applicator pad 510 with the liquid, the distal surface of the applicator pad 510 can be disposed against a wound of the subject. The grip 540 can then be maintained against the applicator pad 510 such that the plate 531 applies distributed pressure to the applicator pad 510 for a period time, as described with respect to the method 200 above. In some embodiments, the bleeding treatment system 500 can be operated with one hand (e.g., the hand of the subject having the wound in need of treatment). For example, the user can place a thumb on the proximal end of the second portion 544 and wrap the remaining fingers of the same hand around the sidewall of the grip 540 (e.g., around the first portion 542), with at least some of the remaining fingers in contact with the grip features 545. While gripping the grip 540 in this configuration, either before or after pressing the applicator pad 510 against the wound, the user can press the second portion 544 toward the applicator pad 510 with the user's thumb to release liquid from the reservoir 550 as described above.

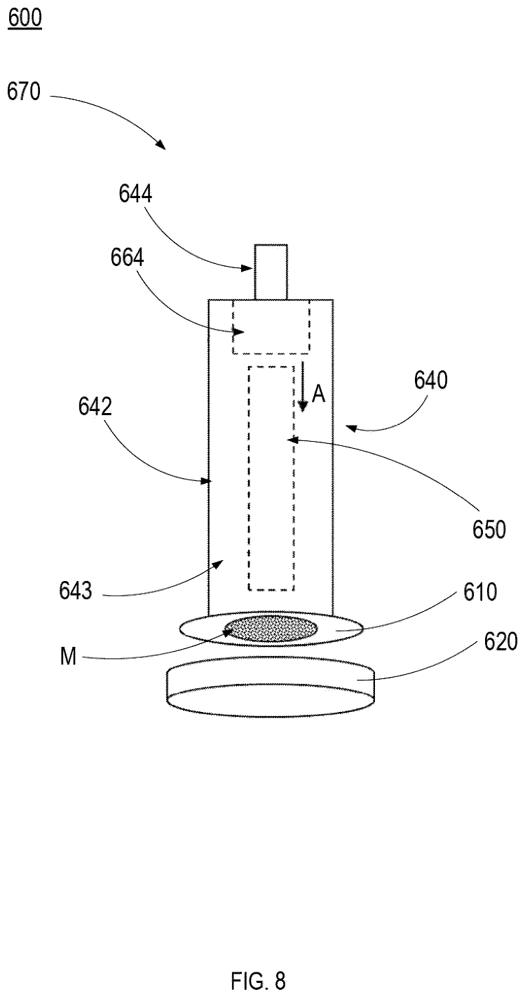

FIG. 8 is a schematic illustration of a bleeding treatment system 600. The bleeding treatment system 600 can be the same or similar in structure and/or function to any of the bleeding treatment systems described herein, such as the bleeding treatment system 400 or the bleeding treatment system 500. For example, the bleeding treatment system 600 includes an applicator device 670, a reservoir 650, and an applicator pad 610. The applicator device 670 includes a cover 620 configured to be releasably coupled to the applicator pad 610 and, optionally, to the distal end of the first portion 642. The applicator device 670 includes a grip 640 including a first portion 642 and a second portion 644. The first portion 642 has an open distal end and an open proximal end such that the reservoir 650 can be inserted into the proximal end of the first portion 642 and disposed in the interior space 643. The second portion 644 can then be coupled to the proximal end of the first portion 642. The second portion 644 includes a projection 664 configured to extend into the internal space 643, contact the reservoir 650, and urge the reservoir and/or liquid within the reservoir toward the applicator pad 610 disposed on the distal end of the first portion 642.

To use the bleeding treatment system 600 to treat a wound and cease bleeding of the wound, the reservoir 650 can be inserted into the interior space 643 of the first portion 642. The second portion 644 can be coupled to a proximal end of the first portion 642. The cover 620 can be removed from the applicator pad 610. The second portion 644 can then be advanced distally in the direction of arrow A to apply pressure to the reservoir 650. Using any suitable reservoir and release mechanism structure, such as any of the reservoir and release mechanism structures described herein, liquid can be released from the reservoir 650 and flow to the applicator pad 610 such that medication M is disposed on the applicator pad 610.

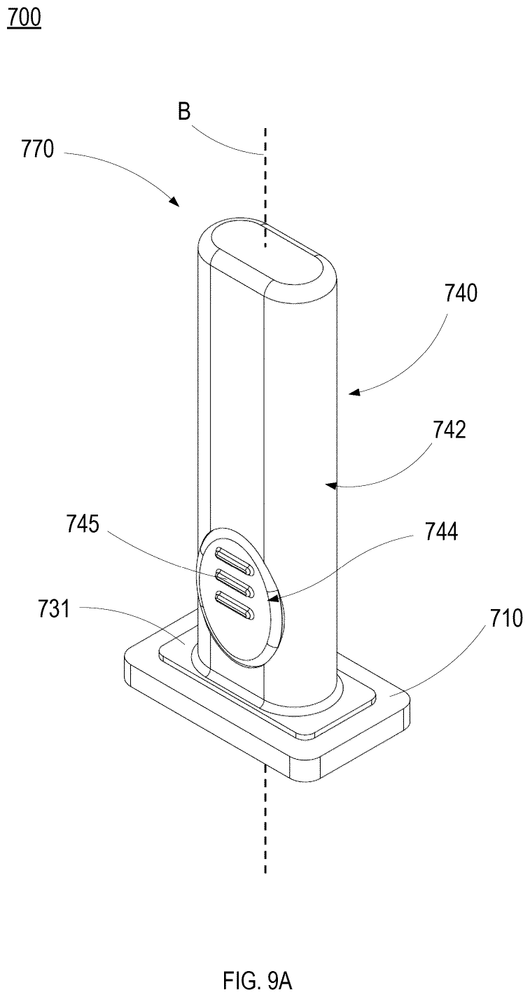

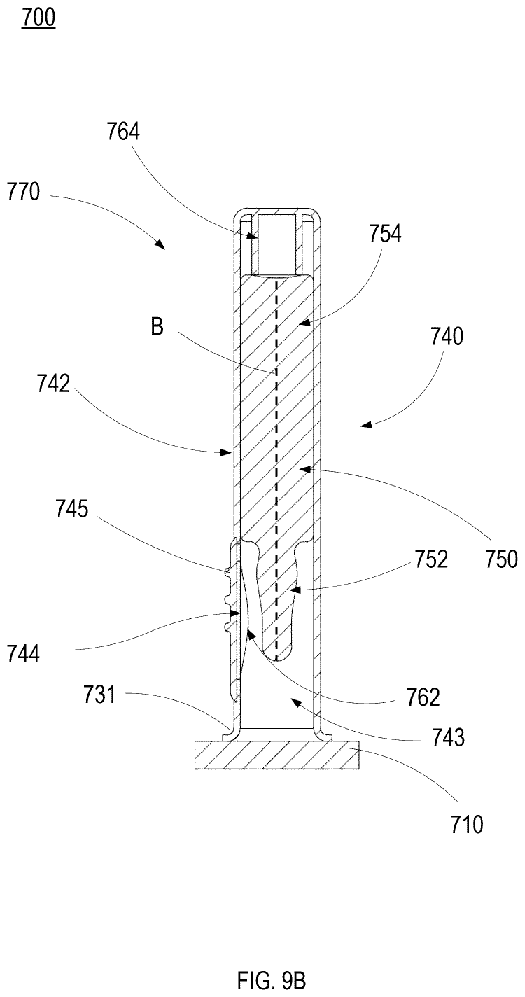

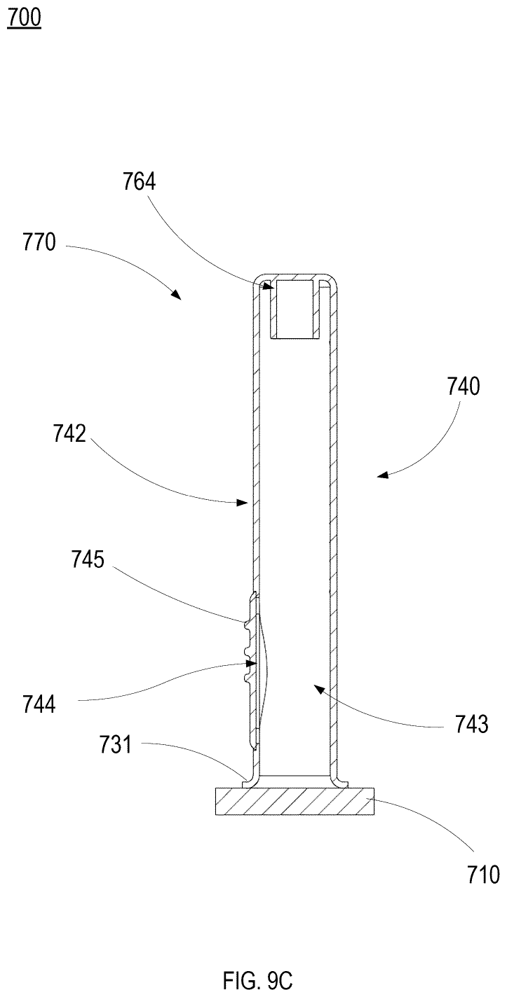

FIGS. 9A and 9B are a perspective view and a cross-sectional view of a bleeding treatment system 700. FIG. 9C is a cross-sectional view of the bleeding treatment system with a reservoir 750 of the bleeding treatment system 700 not shown. The system 700 can be the same or similar in structure and/or function to any of the delivery or treatment systems described herein. For example, the system 700 can include an applicator device 770, a reservoir 750, and an applicator pad 710. The applicator device 770 can include a grip 740 defining an interior space 743 and having an open distal end, a closed proximal end, and extending along a central longitudinal axis B. The grip has a first portion 742 and a second portion 744, the second portion 744 being movable laterally relative to the first portion 742. The reservoir 750 can be formed as an ampoule having a neck portion 752 and a body portion 754. The neck portion 752 can be narrower than the body portion 754 (e.g., can have a smaller diameter). The reservoir 750 can be disposed within the interior space 743 of the grip 740. The reservoir 750 can be oriented such that the neck portion 752 is distal of the body portion 754 and therefore closer to the open end of the grip 740. The interior space 743 can be at least partially defined by an inner surface of a sidewall of the grip 740, and can be defined by a combination of the first portion 742 and the second portion 744. The grip 740 (e.g., the inner surface and/or the outer surface) can be formed as shown in FIG. 8A, having a tubular shape having two opposing flat sides coupled together by two opposing curved sides. In some embodiments, the grip 740 can have any suitable shape, such as is described above with respect to the grip 340.

The first portion 742 can include a plate 731 having a distal or bottom surface extending laterally relative to the central axis B of the grip 740. The applicator pad 710 can be disposed on a distal end of the first portion 742 (e.g., coupled to the distal surface of the plate 431). The first portion 742 can also include one or more retaining portions (not shown) (e.g., flexible tab or bump portions) extending toward the central axis B of the first portion 742 and configured to contact a shoulder of the body portion 754 of the reservoir 750 to retain the reservoir 750 in a proximal position. In some embodiments, rather than or in addition to including one or more retaining portion that contact the shoulder of the body portion 754, the first portion 742 can include retaining portions such as adhesive and/or an inner sidewall in frictional contact with the body portion 754 to retain the body portion 754 in a position in which the neck portion 752 is radially aligned with the second portion 744.

The second portion 744 can be formed as a deformable sidewall portion disposed in an opening defined by the first portion 742. The second portion 744 can optionally include a projecting portion 762 (also referred to as a release mechanism portion) extending and/or deformable laterally toward the central axis B and toward the neck portion 752 of the reservoir 750 in an initial configuration. The second portion 744 can be deformed via being pressed in by a finger of a user such that the projecting portion 762 is urged into sufficient contact with the neck portion 752 to break the neck portion 752 and release the liquid within the reservoir 750. In some embodiments, the second portion 744 can be sufficiently deformable that the second portion 744 can be urged into sufficient contact with the neck portion 752 to break the neck portion 752 such that the second portion 744 operates as the release mechanism portion without including the projecting portion 762. In some embodiments, the second portion 744 can be formed, for example, of silicone or a similar elastomeric material. Although not shown, in some embodiments, the grip 740 can include a ramp portion that can be the same or similar in structure and/or function to ramp portion 562. The ramp portion can be disposed relative to the neck portion 552 and the second portion 744 such that the second portion 744 can be deformed to urge the neck portion 552 into the ramp portion such that the neck portion 552 breaks relative to the body portion 554.

As shown in FIG. 9B, the first portion 742 can include a closed proximal end and an open distal end. The first portion 742 can optionally include a projecting portion 764 coupled to a flexible end of the first portion 742 and configured to contact a proximal end of the reservoir 750 and urge the reservoir 750 distally (or urge liquid to flow from the reservoir 750) when pressed by a user's finger.

To release liquid within the reservoir 750 such that the liquid flows to the applicator pad 710, the second portion 744 can be deformed laterally relative to the neck portion 752 (e.g., via pressing on the second portion 744 with a finger of the user such as a thumb) such that the contact between the second portion 744 and the neck portion 752 of the reservoir 750 causes the neck portion 752 to separate (e.g., break) from the body 754 of the reservoir 750 and allows liquid to flow from the reservoir 750, out of the distal end of the first portion 742, and to the applicator pad 710, resulting in the applicator pad 410 being wetted with the liquid from the reservoir 750.

The second portion 744 can include one or more grip features 745 on an exterior surface of the second portion 744 to improve the grip of the user during handling of the grip 740. The grip features 745 can include any suitable surface feature to increase grip or friction, such as one or more ridges (as shown in FIG. 8A), concave and/or convex curvatures, and/or a textured or course surface.

In some embodiments, the first portion 742 can include a filter to prevent unwanted material (e.g., particles above a certain size and/or glass pieces) from reaching the applicator pad 710. Before or after wetting the applicator pad 710 with the liquid, the distal surface of the applicator pad 710 can be disposed against a wound of the subject. The grip 740 can then be maintained against the applicator pad 710 such that the plate 731 applies distributed pressure to the applicator pad 710 for a period time, as described with respect to the method 200 above. In some embodiments, the first portion 742 and/or the second portion 744 can be deformable (e.g., squeezable) to urge the medication from the reservoir 750 (e.g., after the neck portion 752 has been separated from the body portion 750). In some embodiments, the bleeding treatment system 700 can be operated with one hand (e.g., the hand of the subject having the wound in need of treatment). For example, the user can place a thumb on the second portion 744 and wrap the remaining fingers of the same hand around the sidewall of the grip 740 (e.g., around the first portion 742). While gripping the grip 740 in this configuration, either before or after pressing the applicator pad 710 against the wound, the user can press the second portion 744 toward the neck portion 752 with the user's thumb to release liquid from the reservoir 750 as described above.

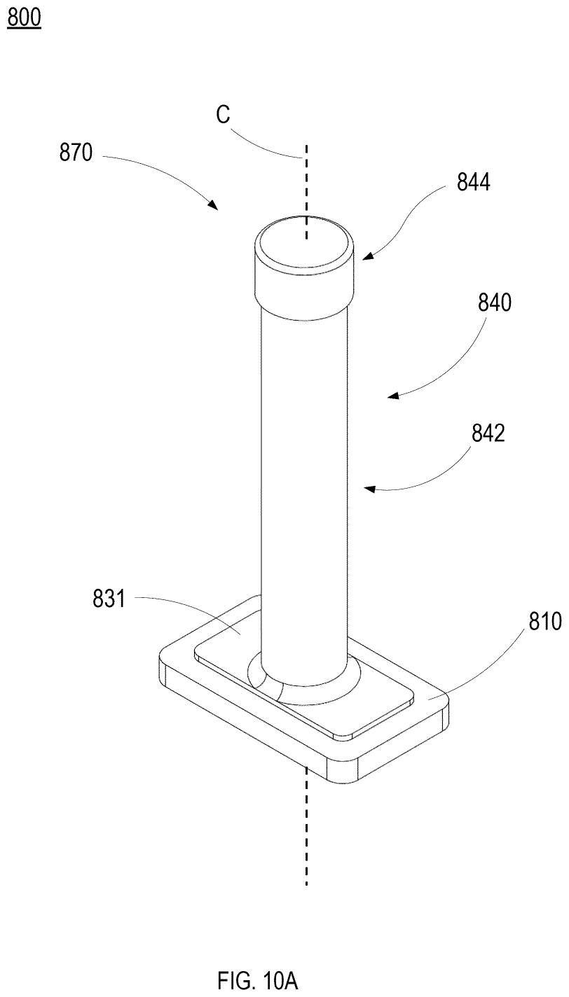

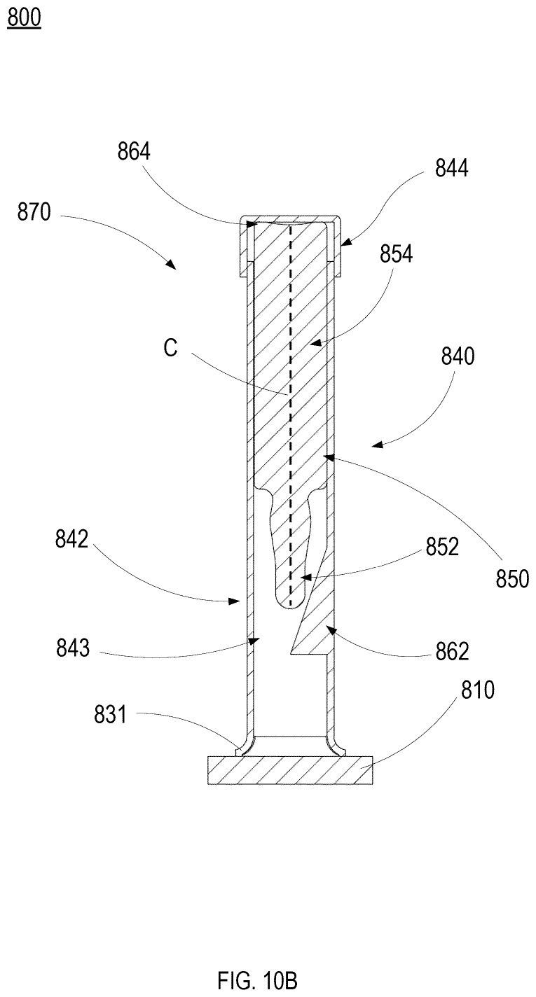

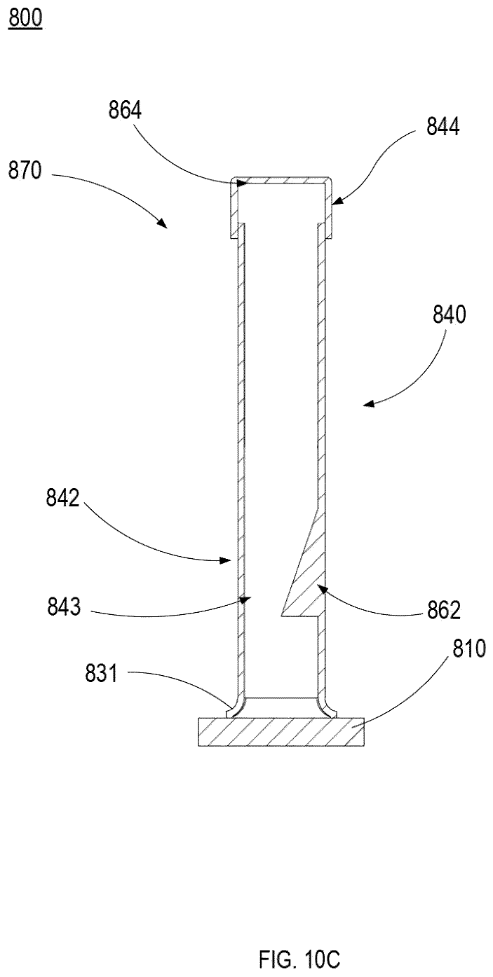

FIGS. 10A and 10B are a perspective view and a cross-sectional view of a bleeding treatment system 800. FIG. 10C is a cross-sectional view of the bleeding treatment system with a reservoir 850 of the bleeding treatment system 800 not shown. The system 800 can be the same or similar in structure and/or function to any of the delivery or treatment systems described herein. For example, the system 800 can include an applicator device 870, a reservoir 850, and an applicator pad 810. The applicator device 870 can include a grip 840 defining an interior space 843 and having an open distal end, a closed proximal end, and extending along a central longitudinal axis C. The grip has a first portion 842 and a second portion 844, the second portion 844 being movable relative to the first portion 842. The reservoir 850 can be formed as an ampoule having a neck portion 852 and a body portion 854. The neck portion 852 can be narrower than the body portion 854 (e.g., can have a smaller diameter). The reservoir 850 can be disposed within the interior space 843 of the grip 840. The reservoir 850 can be oriented such that the neck portion 852 is distal of the body portion 854 and therefore closer to the open end of the grip 840. The interior space 843 can be at least partially defined by an inner surface of a sidewall of the grip 840, and can be defined by the first portion 842 and/or the second portion 844. The grip 840 (e.g., the inner surface and/or the outer surface) can be formed as shown in FIG. 9A, having a cylindrical shape. In some embodiments, the grip 840 can have any suitable shape, such as is described above with respect to the grip 340.

The first portion 842 can include a plate 831 having a distal or bottom surface extending laterally relative to the central axis C of the grip 840. The applicator pad 810 can be disposed on a distal end of the first portion 842 (e.g., coupled to the distal surface of the plate 831). The first portion 842 can include a ramp portion 862 (also referred to as a first release mechanism portion) extending partially into the interior space 843 of the first portion 842 such that the neck portion 852 can contact the ramp portion 862. The first portion 842 can also include one or more retaining portions (not shown) (e.g., flexible tab or bump portions) extending toward the central axis C of the first portion 842 and configured to contact a shoulder of the body portion 854 of the reservoir 850 as shown in FIG. 9B to retain the reservoir 850 in an initial proximal position until sufficient force is applied to a proximal end of the reservoir 850 to advance the reservoir 850 relative to the one or more retaining portions. The first portion 842 can also include one or more grip features (not shown) on an exterior surface of a sidewall of the first portion 842 to improve the grip of the user during handling of the grip 840. The grip features can include any suitable surface feature to increase grip or friction, such as one or more ridges, concave and/or convex curvatures, and/or a textured or course surface.

The second portion 844 can include a distally-facing inner surface 864 (also referred to as a second release mechanism portion). The first portion 842 and the second portion 844 can be coupled such that the second portion 844 can be advanced relative to the first portion 842 towards a distal end of the first portion 842. For example, the first portion 842 can be coupled to the second portion 844 via any suitable coupling mechanism, such as, for example, threads or a slidable friction fit. The second portion 844 can have a larger inner diameter than an outer diameter of the proximal end of the first portion 842 such that the second portion 844 can be disposed in contact with an outer surface of the first portion 842. The reservoir 850 can be disposed in an initial position within the interior space 843 such that the proximal end of the reservoir is near or adjacent to the distally-facing inner surface 864 and can be advanced distally due to the second portion 844 being displaced distally. To release liquid within the reservoir 850 such that the liquid flows to the applicator pad 810, the second portion 844 can be advanced distally relative to the first portion 842 (e.g., via pressing on a proximal end of the second portion 844) such that the inner surface 864 applies a force to the proximal end of the reservoir 850 to urge the reservoir 850 distally such that contact between the neck portion 852 of the reservoir 850 and the ramp 862 causes the neck portion 852 to separate (e.g., break) from a remainder of the reservoir 850 (e.g., the body 854) and allows liquid to flow from the reservoir 850, out of the distal end of the first portion 842, and to the applicator pad 810, resulting in the applicator pad 810 being wetted with the liquid from the reservoir 850.

In some embodiments, the first portion 842 can include a filter to prevent unwanted material (e.g., particles above a certain size and/or glass pieces) from reaching the applicator pad 810. Before or after wetting the applicator pad 810 with the liquid, the distal surface of the applicator pad 810 can be disposed against a wound of the subject. The grip 840 can then be maintained against the applicator pad 810 such that the plate 831 applies distributed pressure to the applicator pad 810 for a period time, as described with respect to the method 200 above. In some embodiments, the first portion 842 and/or the second portion 844 can be deformable (e.g., squeezable) to urge the medication from the reservoir 850. In some embodiments, the bleeding treatment system 800 can be operated with one hand (e.g., the hand of the subject having the wound in need of treatment). For example, the user can place a thumb on the proximal end of the second portion 844 and wrap the remaining fingers of the same hand around the sidewall of the grip 840 (e.g., around the first portion 842). While gripping the grip 840 in this configuration, either before or after pressing the applicator pad 810 against the wound, the user can press the second portion 844 toward the applicator pad 810 with the user's thumb to release liquid from the reservoir 850 as described above.

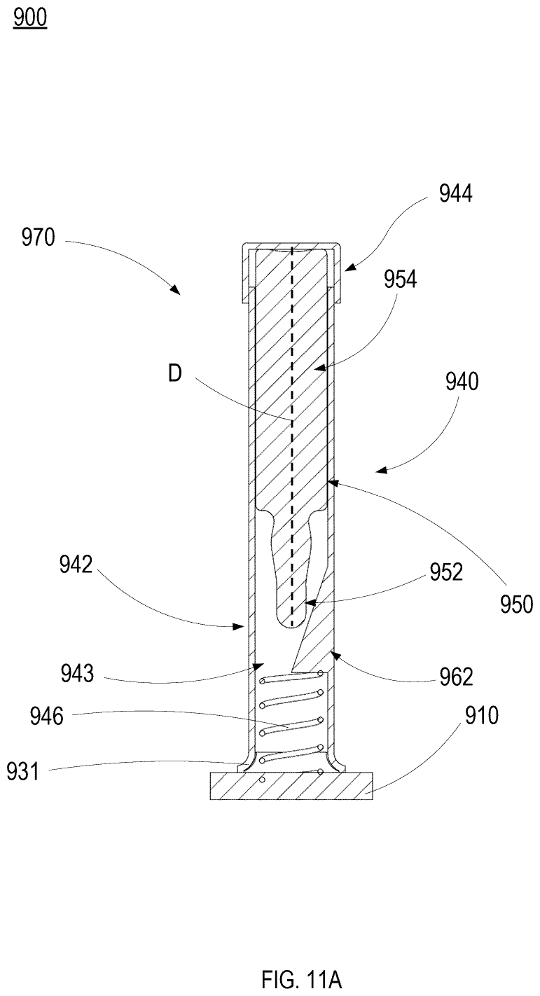

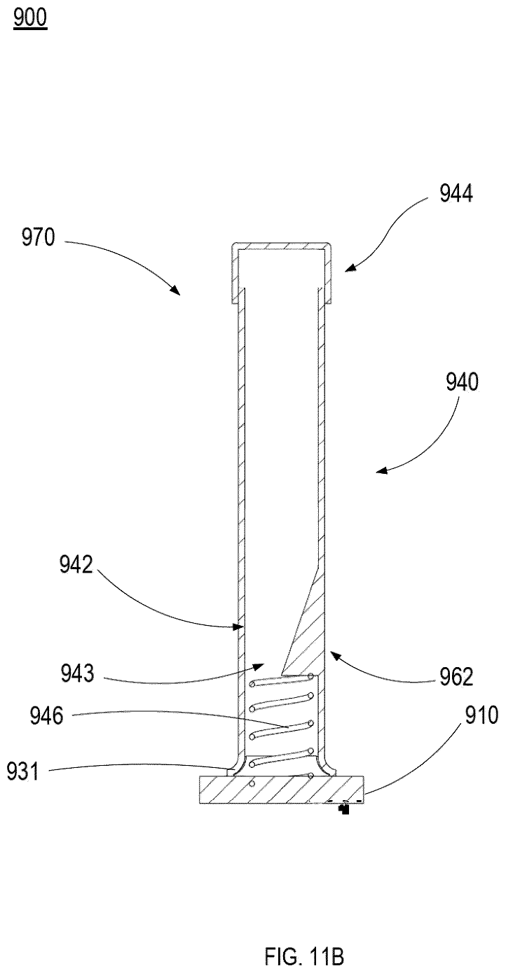

FIGS. 11A and 11B are cross-sectional views of a bleeding treatment system 900 with and without a reservoir 950 shown, respectively. The system 900 can be the same or similar in structure and/or function to any of the delivery or treatment systems described herein. For example, the system 900 can include an applicator device 970, a reservoir 950, and an applicator pad 910. The applicator device 970 can include a grip 940 defining an interior space 943 and having an open distal end, a closed proximal end, and extending along a central longitudinal axis D. The grip has a first portion 942 and a second portion 944, the second portion 944 being movable relative to the first portion 942. The reservoir 950 can be formed as an ampoule having a neck portion 952 and a body portion 954. The neck portion 952 can be narrower than the body portion 954 (e.g., can have a smaller diameter). The reservoir 950 can be disposed within the interior space 943 of the grip 940. The reservoir 950 can be oriented such that the neck portion 952 is distal of the body portion 954 and therefore closer to the open end of the grip 940. The interior space 943 can be at least partially defined by an inner surface of a sidewall of the grip 940, and can be defined by the first portion 942 and/or the second portion 944. The grip 940 (e.g., the inner surface and/or the outer surface) can be formed having a cylindrical shape. In some embodiments, the grip 940 can have any suitable shape, such as is described above with respect to the grip 340.

The first portion 942 can include a plate 931 having a distal or bottom surface extending laterally relative to the central axis D of the grip 940. The applicator pad 910 can be disposed on a distal end of the first portion 942 (e.g., coupled to the distal surface of the plate 931). The first portion 942 can include a ramp portion 962 (also referred to as a first release mechanism portion) extending partially into the interior space 943 of the first portion 942 such that the neck portion 952 can contact the ramp portion 962. The first portion 942 can also include one or more retaining portions (not shown) (e.g., flexible tab or bump portions) extending toward the central axis D of the first portion 942 and configured to contact a shoulder of the body portion 954 of the reservoir 950 to retain the reservoir 950 in an initial proximal position until sufficient force is applied to a proximal end of the reservoir 950 to advance the reservoir 950 relative to the one or more retaining portions. The first portion 942 can also include one or more grip features (not shown) on an exterior surface of a sidewall of the first portion 942 to improve the grip of the user during handling of the grip 940. The grip features can include any suitable surface feature to increase grip or friction, such as one or more ridges, concave and/or convex curvatures, and/or a textured or course surface.

The grip can include a spring portion 946. The first portion 942 and the second portion 944 can be coupled such that the second portion 944 can be advanced relative to the first portion 942 towards a distal end of the first portion 942. For example, the first portion 942 can be coupled to the second portion 944 via any suitable coupling mechanism, such as, for example, threads or a slidable friction fit. The second portion 944 can have a larger inner diameter than an outer diameter of the proximal end of the first portion 942 such that the second portion 944 can be disposed in contact with an outer surface of the first portion 942. The reservoir 950 can be disposed in an initial position within the interior space 943 such that the proximal end of the reservoir is near or adjacent the distally-facing inner surface 964 and can be advanced distally due to the second portion 944 being displaced distally. In some embodiments, the spring portion 946 can be configured to transition from an initial configuration to an expanded or compressed configuration to bring the reservoir 950 into contact with the ramp 962. For example, the spring portion 946 can be activatable to urge the ramp 962 away from the applicator pad 910 and into breaking contact with the neck portion 952. In some embodiments, the spring portion 946 may be activatable to pull or push the reservoir 950 into contact with the ramp 962. The second portion 944 can be engaged with the spring portion 946 such that depression or twisting of the second portion 944 causes the spring portion 946 to activate. Thus, to release liquid within the reservoir 950 such that the liquid flows to the applicator pad 910, the second portion 944 can be twisted or advanced distally relative to the first portion 942 (e.g., via rotating or pressing on a proximal end of the second portion 944) such that the spring portion 946 is released from an initial configuration. The spring portion 946 can then translate the reservoir 950 and/or the ramp 962 such that contact between the neck portion 952 of the reservoir 950 and the ramp 962 causes the neck portion 952 to separate (e.g., break) from a remainder of the reservoir 950 (e.g., the body 954) and allows liquid to flow from the reservoir 950, out of the distal end of the first portion 942, and to the applicator pad 910, resulting in the applicator pad 910 being wetted with the liquid from the reservoir 950.

In some embodiments, the first portion 942 can include a filter to prevent unwanted material (e.g., particles above a certain size and/or glass pieces) from reaching the applicator pad 910. Before or after wetting the applicator pad 910 with the liquid, the distal surface of the applicator pad 910 can be disposed against a wound of the subject. The grip 940 can then be maintained against the applicator pad 910 such that the plate 931 applies distributed pressure to the applicator pad 910 for a period time, as described with respect to the method 200 above. In some embodiments, the first portion 942 and/or the second portion 944 can be deformable (e.g., squeezable) to urge the medication from the reservoir 950. In some embodiments, the bleeding treatment system 900 can be operated with one hand (e.g., the hand of the subject having the wound in need of treatment). For example, the user can place a thumb on the proximal end of the second portion 944 and wrap the remaining fingers of the same hand around the sidewall of the grip 940 (e.g., around the first portion 942). While gripping the grip 940 in this configuration, either before or after pressing the applicator pad 910 against the wound, the user can press the second portion 944 toward the applicator pad 910 with the user's thumb to release liquid from the reservoir 950 as described above.

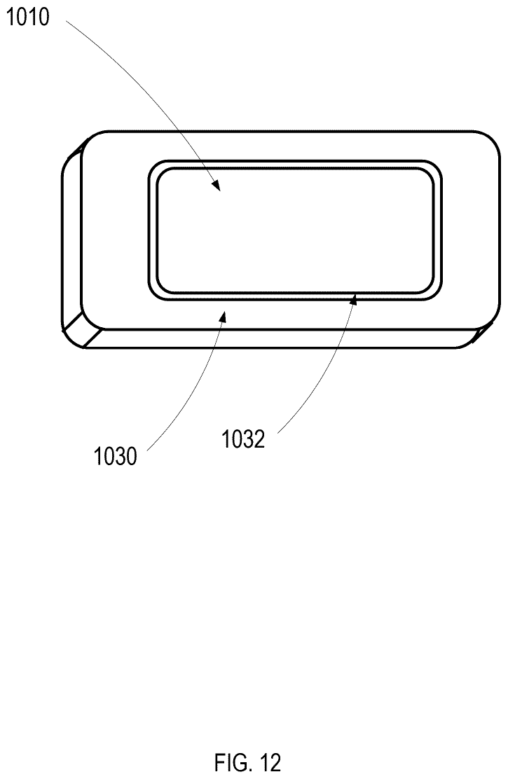

FIG. 12 is a schematic illustration of an applicator pad 1010 in combination with a blood absorption pad 1030, which can be the same or similar in structure and/or function to any of the applicator pads and blood absorption pads, respectively, described herein. As shown, the applicator pad 1010 can be surrounded by the blood absorption pad 1030 such that the applicator pad 1010 and the absorption pad 1030 are concentric. Thus, in some embodiments, the applicator pad 1010 can be shaped, sized, and disposed (e.g., relative to a grip) such that only the applicator pad 1010 and not the blood absorption pad 1030 includes or is contacted by liquid (e.g., medication) from a reservoir. In some embodiments, a liquid impermeable divider 1032 can be disposed between the applicator pad 1010 and the blood absorption pad 1030. In some embodiments, rather than surrounding the applicator pad 1010, the blood absorption pad 1030 can only partially surround or be disposed adjacent to the applicator pad 1010. Although the applicator pad 1010 is shown as having a rectangular shape with curved edges, as described above with respect to applicator pad 110, in some embodiments the applicator pad 1010 can have any suitable shape, such as circular, ovular, or square.

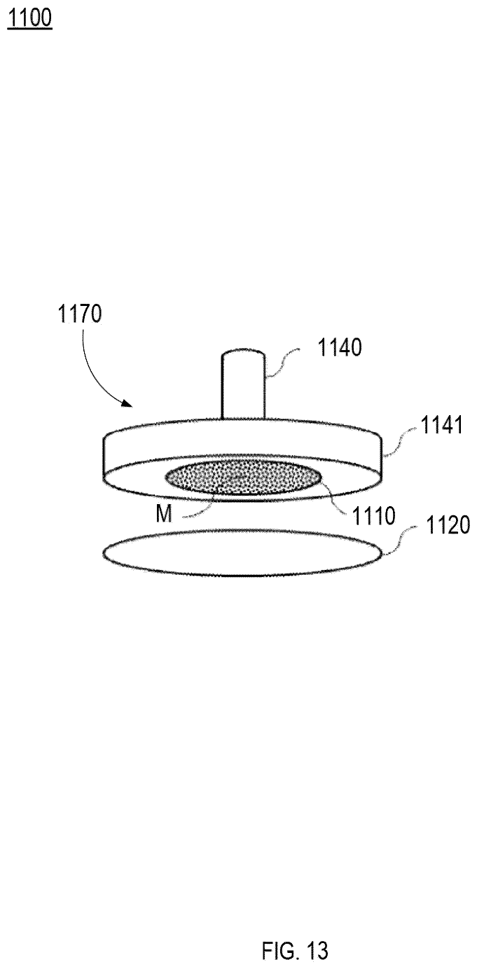

In some embodiments, rather than including a reservoir within a grip of an applicator device, an applicator pad, such as any of the applicator pads described herein, can be prefilled with medication. For example, FIG. 13 is a schematic illustration of a bleeding treatment system 1100. The bleeding treatment system 1100 can be the same or similar in structure and/or function to any of the other bleeding treatment systems described herein. For example, the bleeding treatment system 1100 can include an applicator device 1170 having a grip 1140, an applicator pad 1110 (also referred to as a topical applicator pad), and an applicator seal 1120. In some embodiments, the applicator pad 1110 can be a porous applicator (e.g., a sponge type material) configured to retain liquid medication M and only apply the liquid medication M to a wound site on physical contact. The applicator pad 1110 can be prefilled (e.g., presoaked) with liquid or dry medication M. The grip 1140 can include a plate 1141 having a diameter equal to or larger than the applicator pad 1110 such that the grip 1140 can be used to apply distributed pressure to the applicator pad 1110 when the applicator pad 1110 is in contact with a wound. The plate 1141 can be rigid or semi-rigid or have a surface opposite the surface contacting the applicator pad 1110 that is rigid or semi-rigid to apply uniform pressure to the applicator pad 1110. In some embodiments, the plate 1141 can be flexible. The plate 1141 can function as an insulating mechanism configured to isolate a hand applying pressure from the wound site and the medication in the applicator pad 1110. To use the system 1100, the applicator seal 1120 can be removed from the applicator pad 1110 and the grip 1110 can be used to apply the applicator pad 1110 to a wound such that the medication flows to the wound and to maintain pressure against the wound by applying pressure to the applicator pad 1110.

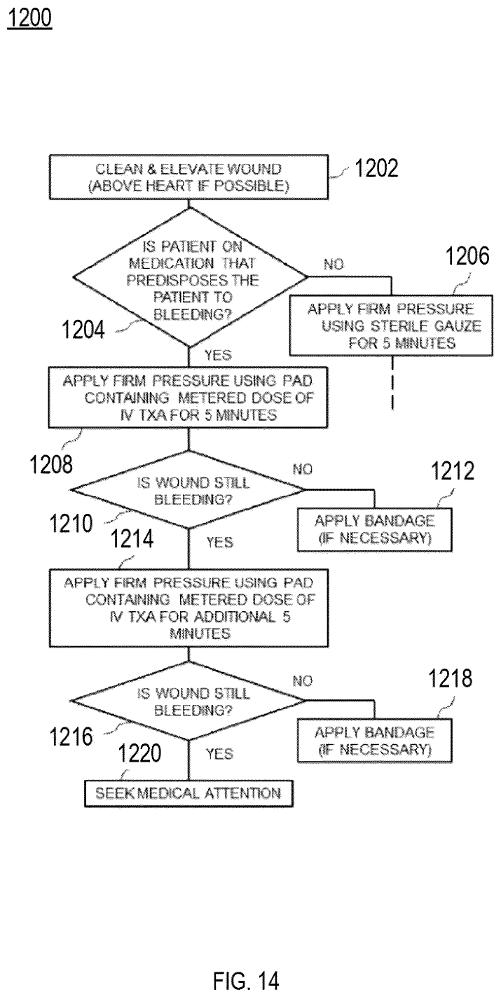

FIG. 14 is a flow chart of a method 1200 of using a bleeding treatment system, such as any of the bleeding treatment systems described herein. The method 1200 can be for reducing bleeding at a wound site of a subject taking a medication that predisposes the subject to increased and/or prolonged bleeding. In some embodiments, the steps of method 1200 can be implemented by the subject, a caregiver, a healthcare provider, a veterinary care provider, etc. In step 1202, the wound can be cleaned and elevated (e.g., by the subject having the wound and/or by a caregiver or clinician) to reduce bleeding. In some embodiments, the wound is cleaned (e.g., with soap and water). In some embodiments, the wound is elevated above the level of the heart of the patient.

In step 1204, whether the subject (e.g., a patient) is on a medication (e.g., an anticoagulant drug or antiplatelet drug) that predisposes the subject to increased and/or prolonged bleeding can be determined. For example, a caregiver or clinician can check to see if such a medication has been taken by the subject within a relevant time period (e.g., by checking a medical record or asking the subject). In some embodiments, step 1204 can be not included and a user can proceed directly to step 1206 described below. In some embodiments, step 1204 can be performed after step 1206 if the wound is still bleeding, and the steps of the method 1200 can proceed from there.

In step 1206, if the subject is determined not to have taken a medication that predisposes the subject to increased and/or prolonged bleeding within a relevant time period, the firm pressure can be applied to the wound using a sterile gauze pad (e.g., by the subject or a caregiver). In some embodiments, the firm pressure can be applied for a first period of time (e.g., a predetermined period of time). In some embodiments, for example, the first period of time can be about five minutes, between about four minutes and about six minutes, less than about four minutes, greater than about five minutes, greater than about six minutes. After the first period of time, the pressure can be removed. If the wound is no longer bleeding, the subject or a caregiver can apply a bandage to the wound. If the wound is still bleeding, then step 1208 can be performed as described below and/or medical attention may be sought.

In step 1208, if the subject is determined to have taken a medication that predisposes the subject to increased and/or prolonged bleeding within a relevant time period, or if applying firm pressure for the first period of time as described in step 1206 has not caused bleeding to stop, firm pressure can be applied to the wound using a topical applicator pad containing a metered dose of medication (e.g., TXA such as intravenous (IV) TXA) for a second period of time (e.g., a second predetermined period of time) (e.g., five minutes) such that the medication contacts the wound. In some embodiments, the second period of time can be greater than the first period of time. In some embodiments, the second period of time can be less than the first period of time. In some embodiments, the metered dose can be between three and ten mL and/or include between about 300 and about 1000 mg of medication.

In step 1210, the pressure can be removed by the subject or a caregiver (e.g., via removing the applicator pad) and the wound can be checked to see if the wound is still bleeding. In step 1212, if the wound is no longer bleeding, a bandage can be applied to the wound by the subject or a caregiver. In step 1214, if the wound is still bleeding, the subject or a caregiver can reapply firm pressure to the wound using the topical applicator pad for an additional predetermined time period (e.g., for an additional five minutes). In some embodiments, a new topical applicator pad (containing a metered dose or not containing a metered dose of medication) may be used. In step 1216, the pressure can be removed and checked to see if the wound is still bleeding. In step 1218, if the wound is no longer bleeding, a bandage can be applied to the wound if needed. In step 1220, if the wound is still bleeding, the subject can seek medical attention (with the help of a caregiver if needed).

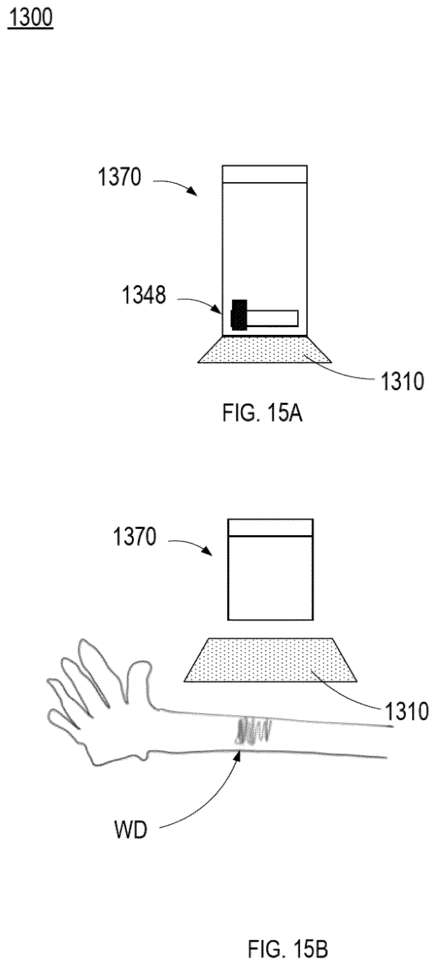

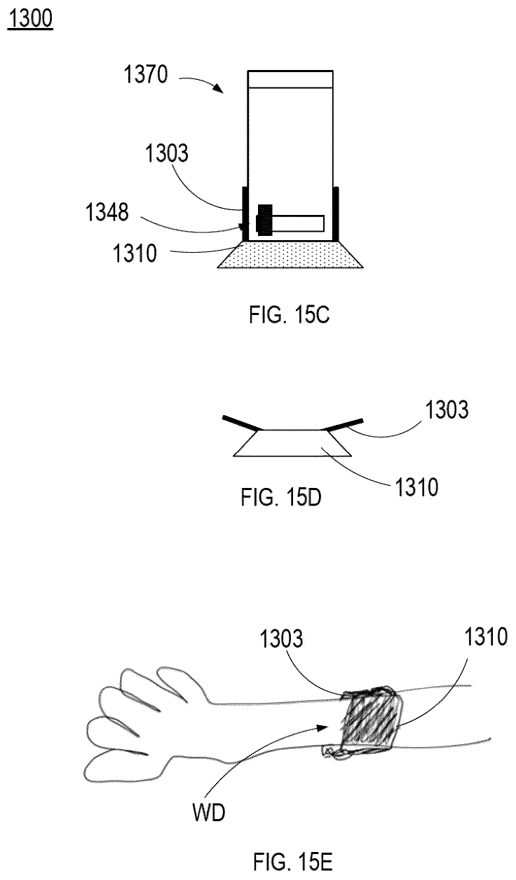

FIGS. 15A-15E are schematic illustrations of a bleeding treatment system 1300 in various stages of use. The system 1300 can be the same or similar in structure and/or function to any of the systems described herein. For example, the system 1300 can include an applicator device 1370, a reservoir (not shown), and an applicator pad 1310. As shown in FIG. 15A, the applicator pad 1310 can be coupled to the applicator device 1370 via a pad connector 1348. The pad connector 1348 can include any suitable coupling mechanism, such as any of the coupling mechanisms and pad connector described herein. For example, the applicator device 1370 can include a sliding mechanism or a latch disengageable with an engagement feature associated with the applicator pad 1310 such that the engagement feature associated with the applicator pad 1310 can be released from the applicator device 1370 before or after being applied to a wound WD, as shown in FIG. 15B.

In some embodiments, the applicator pad 1310 can be coupled to the applicator device 1370 via adhesive strips 1303 that are releasable from the applicator device 1370 via, for example, the pad connector 1348 which can operate as a release mechanism to allow the adhesive strips 1303 to contact skin of a patient to secure the pad connector 1348 to a wound WD. Thus, in the first configuration shown in FIG. 15C, the applicator pad 1310 can be coupled to the applicator device 1370. As shown in FIG. 15D, the applicator pad 1310 and the adhesive strips 1303 can be decoupled from the applicator device 1370. The applicator pad 1310 can then be secured to the subject at the location of the wound via the adhesive strips 1303.

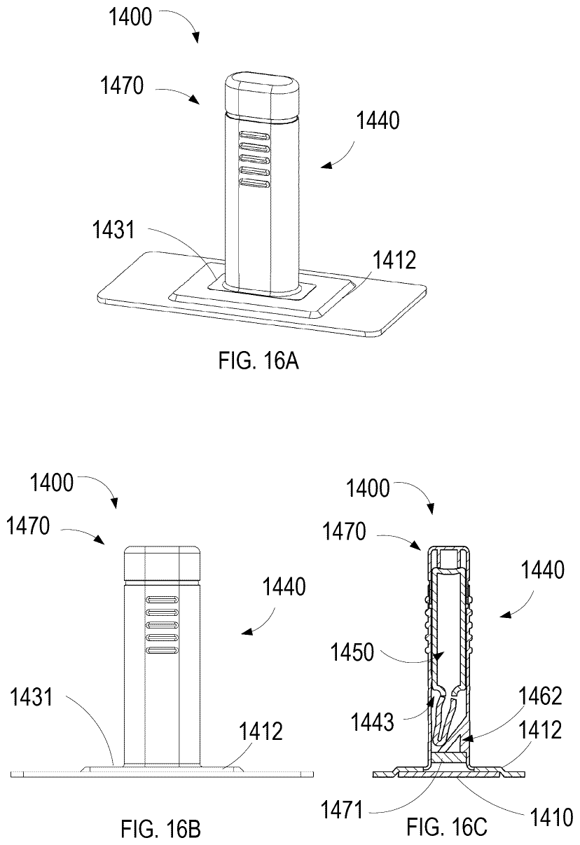

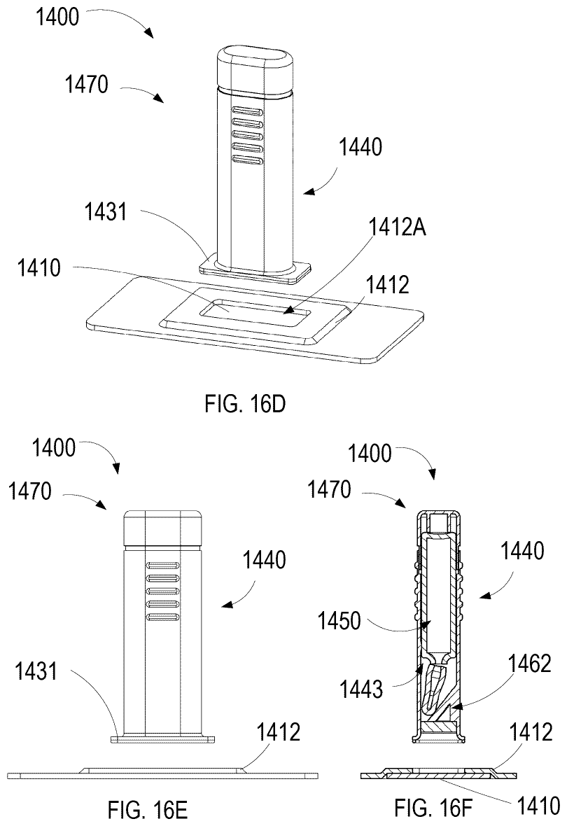

FIGS. 16A-16C are schematic illustrations of a perspective, front, and cross-sectional view, respectively, of a bleeding treatment system 1400 in wetting configuration in which a reservoir 1450 has been broken to release the contents of the reservoir 1450. FIGS. 16D-16F are schematic illustrations of a perspective, front, and cross-sectional view, respectively, of the bleeding treatment system 1400 in a separated configuration in which a grip 1440 of the system 1400 has been decoupled from an applicator pad 1410. The system 1400 can be the same or similar in structure and/or function to any of the delivery or treatment systems described herein, such as, for example, the bleeding treatment system 500. For example, the system 1400 includes an applicator device 1470, the reservoir 1450, and the applicator pad 1410. The applicator device 1470 includes a grip 1440 defining an interior space 1443 and having an open distal end, a closed proximal end, and extending along a central longitudinal axis. The applicator device 1470 includes a filter 1471 (e.g., disposed between a ramp 1462 of the grip 1440 and the distal opening of the grip 1440) to prevent unwanted material (e.g., particles above a certain size and/or glass pieces) from reaching the applicator pad 1410.

As shown in FIGS. 16A-16F, the system 1400 can include a bandage 1412 coupled to a portion of an upper surface of the applicator pad 1410 and extending beyond an outer perimeter of the applicator pad 1410. Although shown as surrounding a periphery of the applicator pad 1410, in some embodiments the bandage can extend beyond opposing portions or sides of the applicator pad 1410 but not in all directions. The bandage 1412 can define one or more openings 1412A adjacent the upper surface of the applicator pad 1410 such that fluid can flow through the openings and into wetting contact with the applicator pad 1410. In some embodiments, the one or more openings 1412A can collectively have a surface area and perimeter equal to or smaller than the surface area and perimeter of the distal opening of the grip 1440. In some embodiments, the bandage 1412 can include a liquid permeable layer or portion covering the one or more openings 1412A. In some embodiments, the bandage 1412 can be adhesively coupled to the upper surface of the applicator pad 1410 via one or more adhesive portions.

The system 1400 can be placed on a surface of a subject with the applicator pad 1410 and the bandage 1412 in contact with the surface, the applicator pad 1410 contacting a wound of the subject and the bandage 1412 surrounding the applicator pad 1410. The applicator device 1470 can be transitioned from an initial configuration to the wetting configuration shown in FIGS. 16A-16C via the same steps as described with respect to the system 500. After the contents of the reservoir 1450 are released from the reservoir 1450 as shown in FIG. 16C, the contents can flow through the filter 1471, through the one or more openings 1412A in the bandage 1412, to the applicator pad 1410, and into contact with the wound. The grip 1440 can be used to apply pressure to the applicator pad 1410 and thus the wound (e.g., before and/or after wetting the applicator pad 1410) by applying a downward pressure to the grip 1440 with the distal end of the grip 1440 and/or the plate 1431 contacting the applicator pad 1410.

In some embodiments, the applicator pad 1410 and the bandage 1412 can be left on the skin of the subject such that the applicator pad 1410 remains in contact with the wound, and the grip 1440 can be decoupled from the applicator pad 1410 and the bandage 1412 such that the applicator pad 1410 is not displaced relative to the wound during the decoupling. As shown in FIGS. 16D-16F, after decoupling the grip 1440 from the applicator pad 1410 and/or the bandage 1412, the grip 1440 can be discarded, leaving the applicator pad 1410 and the bandage 1412 in place on the wound and skin of the subject.

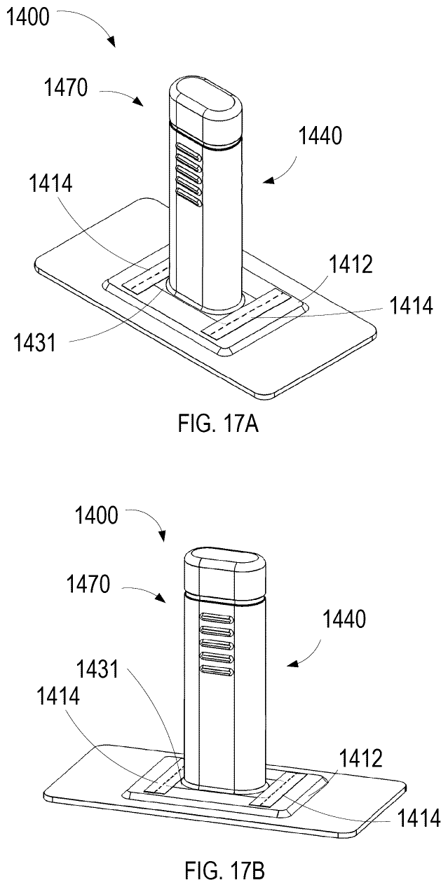

The grip 1440 can be coupled to and/or decoupled from the applicator pad 1410 and/or the bandage 1412 via any suitable decoupling method described herein. For example, as shown in FIGS. 17A and 17B, which are two different perspective views of the system 1400 including two adhesive strips 1414 (also referred to as pad connectors), the applicator device 1470 can be coupled to the bandage 1412 via removable adhesive strips 1414. The adhesive strips 1414 can be disposed in contact with an upper surface of the plate 1431 on either side of the grip 1440 and can each have ends in contact with the bandage 1412 such that the adhesive strips 1414 secure the grip 1440 to the bandage 1412 via the plate 1431. In some embodiments, the adhesive strips 1414 can be flexible but have low or no elasticity. To separate the grip 1440 from the bandage 1412 and the applicator pad 1410, each of the adhesive strips 1414 can be peeled away from the bandage 1412 to release the bandage 1412 from the plate 1431.

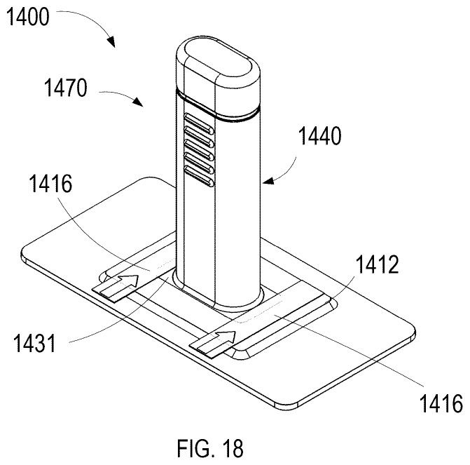

In some embodiments, as shown in FIG. 18, rather than including adhesive strips that are peelable and have low or no elasticity, the system 1400 can include adhesive strips 1416 that have high elasticity and can be decoupled from the bandage 1412 and/or the plate 1431 via stretching a free end of each of the adhesive strips 1416 away from but within or near a plane containing the portion of the adhesive strip 1416 (also referred to as pad connectors) adhesively coupled to the bandage 1412 and/or the plate 1431 such that the adhesive strip 1416 is deformed and the adhesive contact between the adhesive strip 1416 and the bandage 1412 and/or the plate 1431 is broken. For example, the adhesive strips 1416 can include a synthetic rubber resin adhesive.

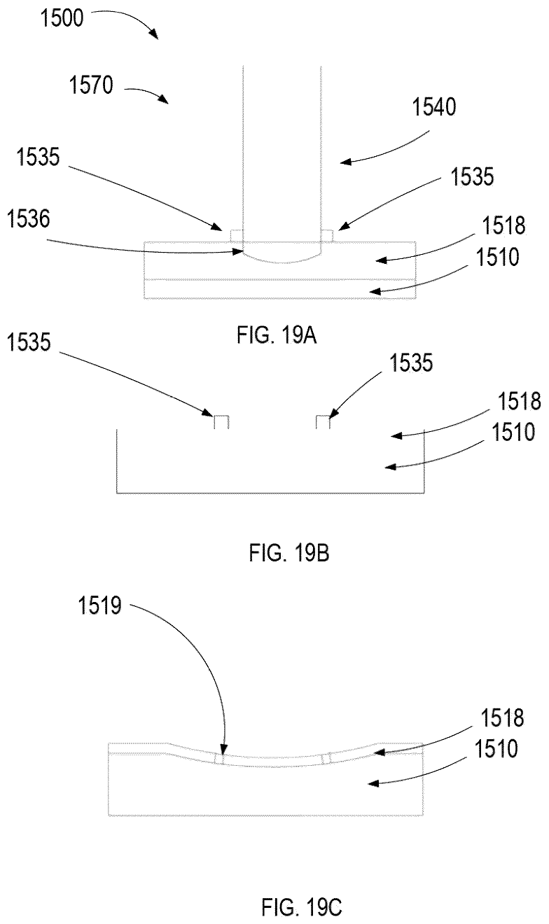

In some embodiments, an applicator pad can include or be coupled to a rigid or semi-rigid backing portion that is configured to be releasably engaged by a portion of an applicator device. For example, FIG. 19A is a schematic illustration of a system 1500 that can be the same or similar in structure and/or function to any of the other systems described herein. The system 1500 can include an applicator device 1570 including a grip 1540, an applicator pad 1510, and a reservoir (not identified) that is included within the applicator pad 1510 and/or within the grip 1540. As shown in FIG. 19B, the applicator pad 1510 can be coupled (e.g., via adhesive) to a backing portion 1518 that can be rigid or more rigid than the applicator pad 1510. The backing portion 1518 can define one or more openings (not shown in FIGS. 19A and 19B) such that fluid can flow from the reservoir within the grip 1540 to the applicator pad 1510 to wet the applicator pad 1510. The backing portion 1518 can include one or more pad retainer portions 1535 configured to be received by or otherwise coupled to complementary pad retainer portions (not shown) of the grip 1540 such that the grip 1540 can be adhered, clipped, latched, frictionally fit, hook-and-loop fastened, buttoned, or otherwise releasably coupled via any suitable coupling mechanism to the backing portion 1518. After applying pressure to the backing portion 1518 to push the applicator pad 1510 against the wound, the grip 1540 can be decoupled from the backing portion 1518 by uncoupling the complementary pad retainer portions. As shown in FIG. 19A, the grip 1540 can include a convex distal surface 1536 configured to apply targeted and increased pressure to the wound when a user presses the grip 1540 into the applicator pad 1510 coupled to the wound. The convex distal surface 1536 can include one or more openings to allow fluid flow through the convex distal surface 1536.

As shown in FIG. 19C, in some embodiments, the backing portion 1518 can be curved and rigid such that the convex distal surface 1536 of the grip 1540 can be seated in a concave upper surface of the backing portion 1518 during wetting of the applicator pad 1510 and/or during application of pressure to the applicator pad 1510 and, thus, the wound. As shown in FIG. 19C, the backing portion 1518 can define one or more openings 1519 such that fluid can flow from the reservoir within the grip 1540 to the applicator pad 1510 to wet the applicator pad 1510.

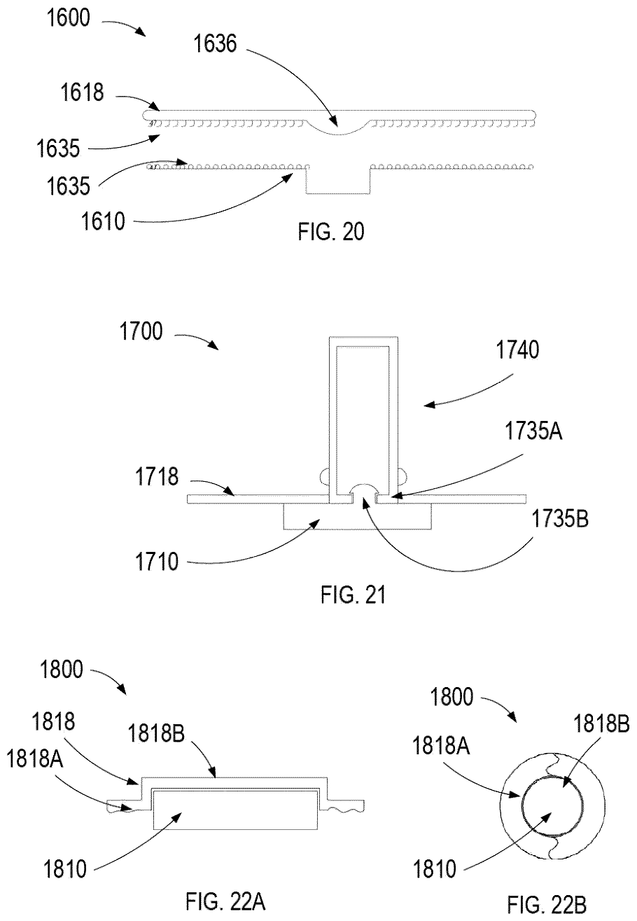

As shown in FIG. 20, which is a schematic illustration of a system 1600, in some embodiments a backing portion 1618 can be releasably coupled to an applicator pad 1610 via an attachment such as a hook-and-loop fastener 1635 (e.g., Velcro®) or releasable adhesive (not shown). The backing portion 1618 can include a convex distal surface 1636 configured to apply pressure to the portion of the applicator pad 1610 in contact with a wound when the applicator pad 1610 contacts a wound and the backing portion 1618 is coupled to the applicator pad 1610. After hemostasis occurs, the backing portion 1618 can be removed from the applicator pad 1610, leaving the applicator pad 1610 in place relative to the wound.

FIG. 21 is a schematic illustration of a system 1700. The system 1700 can be the same or similar in structure and/or function to any of the systems described herein. For example, the system 1700 includes a grip 1740 and an applicator pad 1710. The applicator pad 1710 can be coupled to a backing portion 1718 that may be configured as a bandage. The backing portion 1718 can include an adhesive covered by a peel-away adhesive liner disposed on a distal surface of the backing portion. The adhesive liner can be peeled away prior to placing the backing portion 1718 on a subject's skin so that the backing portion 1718 can be adhered to the skin via the adhesive. In some embodiments, the backing portion 1718 can extend laterally from the grip 1740 prior to disposal on the skin as shown in FIG. 21. In some embodiments, the backing portion 1718 can be flush with the grip 1740 (e.g., extending upward along the sidewall of the grip 1740) and then folded down to adhere to the skin. In some embodiments, the backing portion 1718 can fully surround and/or extend circumferentially away from the applicator pad 1710. In some embodiments, the backing portion 1718 can extend along one axis of the applicator pad 1710.

The backing portion 1718 and/or the applicator pad 1710 can include or be coupled to a pad retainer portion 1735B extending away from the applicator pad 1710 and configured to be releasably grasped by a pad retainer portion 1735A of the grip 1740. The pad retainer portion 1735A of the grip 1740 can include, for example, flexible arms configured to engage with a recess of the pad retainer portion 1735B such that the grip 1740 and the applicator pad 1710 are coupled (e.g., during wetting and/or applying pressure to the wound). To separate the grip 1740 from the applicator pad 1710, the flexible arms can be pulled away from the recess of the pad retainer portion 1735B (e.g., via pulling on tabs extending from an outer surface of the grip 1740 or by squeezing engagement mechanisms (e.g., semicircular buttons) near or adjacent to the distal end of the grip 1740 to activate a release mechanism such that the pad retainer portion 1735B is released) and the grip 1740 can be removed from the pad retainer portion 1735B.

FIGS. 22A and 22B are cross-sectional and top views of a schematic illustration of a system 1800. The system 1800 can be the same or similar in structure and/or function to any of the systems described herein. For example, the system 1800 includes a semi-rigid backing portion 1818 that may be configured as a bandage and an applicator pad 1810. As shown, the semi-rigid backing portion 1818 can include a foam ring 1818A surrounding the applicator pad 1810 and a backing layer 1818B disposed against the upper surface of the applicator pad 1810 and coupled to the foam ring 1818A. The foam ring 1818A can adhesively couple the backing portion 1818 and the applicator pad 1810 to the surface of the skin of the subject and apply compressive pressure to the applicator pad 1810 to urge the applicator pad 1810 against a wound. In some embodiments, the pressure applied to the applicator pad 1810 can activated release of medication(s) from the applicator pad 1810.

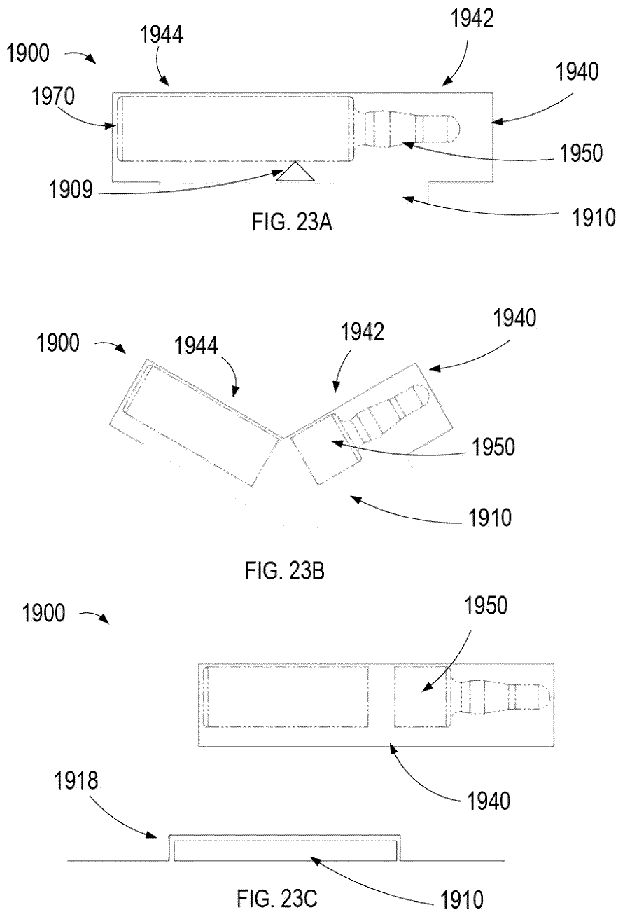

In some embodiments, the contents of a reservoir can be configured to flow from the reservoir to an applicator pad through a sidewall of a grip containing the reservoir. For example, FIGS. 23A-23C are schematic illustrations of a system 1900 in various stages of operation. The system 1900 can be the same or similar in structure and/or function to any of the systems described herein. For example, the system 1900 includes an applicator device 1970, a reservoir 1950, and an applicator pad 1910. The applicator device 1970 can include a grip 1940 defining an interior space within which the reservoir 1950 can be disposed. As shown, the grip 1940 can be disposed horizontally such that the applicator pad 1910 is coupled to a sidewall of the grip 1940 and the grip 1940 and/or the reservoir 1950 are elongated along a longitudinal axis disposed substantially parallel to the applicator pad 1910 in the initial configuration shown in FIG. 23A.

The grip 1940 can include a first portion 1942 and a second portion 1944 collectively defining the interior space within which the reservoir 1950 is disposed. The first portion 1942 can be configured to be bent relative to the second portion 1944 as shown in FIG. 23B to cause the reservoir 1950 to break and the contents of the reservoir 1950 to flow from the reservoir 1950 to the applicator pad 1910. For example, the grip 1940 can be bendable such that a first end of the grip 1940 can be rotated relative to a second end of the grip 1940, causing an inner surface of the grip 1940 to apply pressure to the reservoir 1950 to snap, crush, or otherwise break the reservoir 1950. In some embodiments, the grip 1940 can include a bendable hinge or notched feature near a midpoint of the grip 1940 such that the first portion 1942 can be rotated relative to the second portion 1944 about the hinge or notch to cause the reservoir 1950 to break. In some embodiments, the grip 1940 can include a force concentrating component 1909 such as a tipped projection or a steel ball that can project from an inner surface of the grip 1940 such that bending the grip 1940 pushes the force concentrating component 1909 into breaking contact with a portion of the reservoir 1950, causing the reservoir 1950 to break. In some embodiments, the force concentrating component 1909 can be aligned with a weakened or pre-scored portion of the reservoir such that the reservoir will be preferentially broken by the force concentrating component 1909 at the weakened or pre-scored portion. In some embodiments, a subject or caregiver can use two hands to bend the first portion 1942 relative to the second portion 1944 to break the reservoir 1950. In some embodiments, a subject or caregiver can place the system 1900 on a hard surface (e.g., a table) and apply a downward orthogonal force against the grip 1940 such that the reservoir 1950 is urged into breaking contact with the force concentrating component 1909 and the contents of the reservoir 1950 are released. In some embodiments, a subject or caregiver can place the system 1900 directly against the wound with the applicator pad 1910 against the wound, and can apply a force orthogonal to the wound to the grip 1940 such that the reservoir 1950 is urged into breaking contact with the force concentrating component 1909 and the contents of the reservoir 1950 are released. Thus, in some embodiments, a user can apply pressure to the grip 1940 using a palm of the user's hand, and that pressure can be concentrated to a particular location on the reservoir 1950 by the force concentrating component 1909 to break the reservoir 1950.

After the reservoir 1950 is broken, the contents of the reservoir 1950 can flow from the reservoir 1950 to the applicator pad 1910 through a sidewall of the grip 1940. For example, the grip 1940 can define one or more openings through which fluid can flow to the applicator pad 1910, which may be coupled or releasably coupled to a sidewall of the grip 1940 and aligned with the openings. In some embodiments, the grip 1940 can include a filter (not shown) disposed between the reservoir 1950 and the applicator pad 1910 to prevent unwanted material (e.g., particles above a certain size and/or glass pieces) from reaching the applicator pad 1910. For example, the filter can be disposed within or adjacent to the one or more openings defined by the grip 1940.

In some embodiments, the grip 1940 can be releasably coupled to the applicator pad 1910 using any of the releasable coupling mechanism described herein such that, as shown in FIG. 23C, the grip 1940 can be decoupled from the applicator pad 1910 after wetting the applicator pad 1910. For example, before or after breaking the reservoir 1950 to wet the applicator pad 1910, the applicator pad 1910 can be disposed in contact with a target wound. After applying pressure to the grip 1940 to break the reservoir 1950 and wet the applicator pad 1910, the grip 1940 can be pressed against the applicator pad 1910 to apply pressure to the wound. After a period of time, the grip 1940 can be decoupled from the applicator pad 1910, leaving the applicator pad 1910 disposed in contact with the wound. In some embodiments, the applicator pad 1910 can include an adhesive portion on a skin-contacting surface of the applicator pad 1910 (e.g., adjacent to a perimeter of the skin-contacting surface) to retain the applicator pad 1910 in place relative to the wound. In some embodiments, adhesive tabs can be applied to the applicator pad 1910 and the skin of the subject to retain the applicator pad 1910 in place relative to the wound. In some embodiments, as shown in FIG. 23C, a backing portion 1918, which may be the same or similar as any of the backing portions or bandages described herein, can be placed over the top of the applicator pad 1910 to apply compressive pressure to the applicator pad 1910 and, thus, the wound, and/or to maintain the applicator pad 1910 in place relative to the wound.

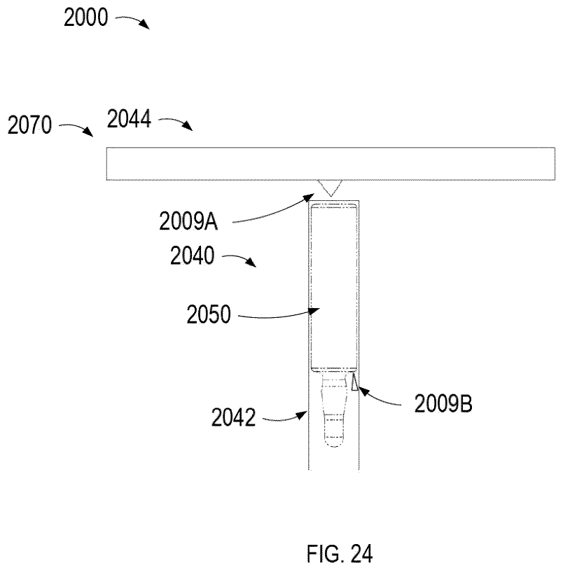

In some embodiments, a force concentrating component can be disposed on a first portion or a second portion of a grip and the second portion of the grip can be moved (e.g., rotated and/or translated) relative to a first portion of the grip containing a reservoir to break the reservoir due to contact between the reservoir and the force concentrating component. For example, FIG. 24 is a schematic illustration of a system 2000. The system 2000 can be the same or similar in structure and/or function to any of the systems described herein. For example, the system 2000 includes a reservoir 2050, an applicator device 2070, and an applicator pad (not shown). The applicator device 2070 includes a grip 2040 including a first portion 2042 and a second portion 2044. The first portion 2042 can define an interior space within which the reservoir 2050 can be disposed. The second portion 2044 can be configured to rotate and advance relative to the first portion 2042. For example, the second portion 2044 can be coupled to the first portion 2042 via a threaded connection. A first force concentrating component 2009A can be disposed on a reservoir-facing surface of the second portion 2044 and/or a second force concentrating component 2009B can be disposed on a reservoir-facing surface of the first portion 2042 (e.g., on a sidewall or a ramp or projection extending from the sidewall). In some embodiments, rotation of the second portion 2044 can cause the second portion 2044 to advance toward the reservoir and apply pressure to the reservoir 2050 with the first force concentrating component 2009A until the reservoir 2050 breaks. In some embodiments, rotation of the second portion 2044 can cause the second portion 2044 to advance toward the reservoir and/or advance the reservoir such that the reservoir 2050 is urged against the second force concentrating component 2009B until the reservoir 2050 breaks. In some embodiments, the second force concentrating component 2009B can be disposed such that the second force concentrating component 2009B will contact a shoulder of the reservoir 2050 and break the reservoir 2050 at the shoulder. In some embodiments, an air vent (not shown) can be included to facilitate fluid travel from the reservoir 2050 toward the applicator pad (not shown).

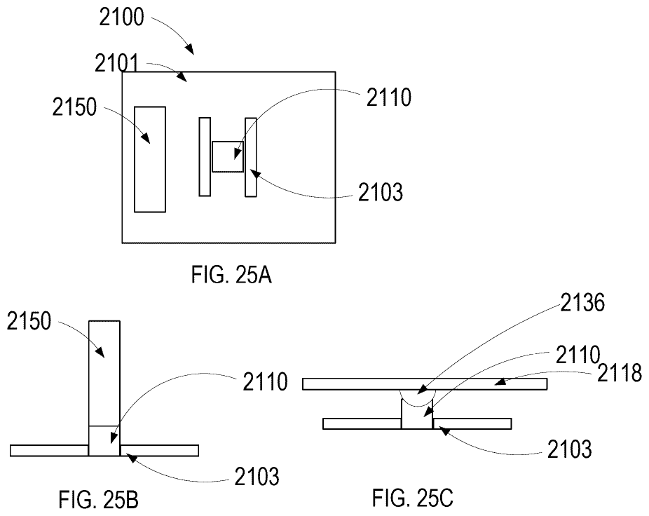

In some embodiments, an applicator pad can be prepared (e.g., wetted) in a tray included in a kit prior to being applied to a target wound or treatment area of a subject. For example, FIGS. 25A-25C are schematic illustrations of a sequence of steps performed using a kit 2100. The kit 2100 can include any of the systems used according to any of the methods described herein. For example, the kit 2100 can include an applicator pad 2110 and a reservoir 2150 containing fluid. As shown in FIG. 25A, the kit 2100 can include a tray 2101 housing the applicator pad 2110 and the reservoir 2150. The fluid in the reservoir 2150 can include a medication and/or a medication activating agent such as saline. As shown in FIG. 25A, the applicator pad 2110 can optionally include or be coupled to adhesive strips 2103 configured to attach the applicator pad 2110 to skin of a subject. As shown in FIG. 25B, the fluid can be transferred from the reservoir 2150 to the applicator pad 2110 to transfer medication to the applicator pad 2110 and/or to combine with dry medication predisposed in the applicator pad 2110. The reservoir 2150 can be similar to any of the reservoirs described herein. Furthermore, the fluid can be transferred from the reservoir 2150 using any of the applicator devices described herein. As shown in FIG. 25C, a backing portion 2118 (e.g., a bandage) can be coupled to the applicator pad 2110 (e.g., via adhesive). The backing portion 2118 can include a convex distal surface 2136 configured to be coupled to the applicator pad 2110 to apply targeted and increased pressure to the wound via the applicator pad 2110. The applicator pad 2110 can then be disposed in contact with the wound with the backing portion 2118 being coupled to the skin surrounding the wound. In some embodiments, the backing portion 2118 can be removed after a predetermined period of time (e.g., four, five, six, or ten minutes).

In some embodiments, an applicator device such as any of the applicator devices described herein can be included in the kit 2100 can used to release the contents of the reservoir. The applicator device can then be used to apply the applicator pad 2110 to the wound and to apply pressure to the wound, or the applicator pad 2110 can optionally be separated from the applicator device (e.g., in the tray 2101) and the applicator pad 2110 can be applied to the wound and pressed against the wound using fingers and/or a palm of the subject or caregiver. Optionally, the backing portion 2118 can be applied to the applicator pad 2110 before or after applying the applicator pad 2110 to the wound.

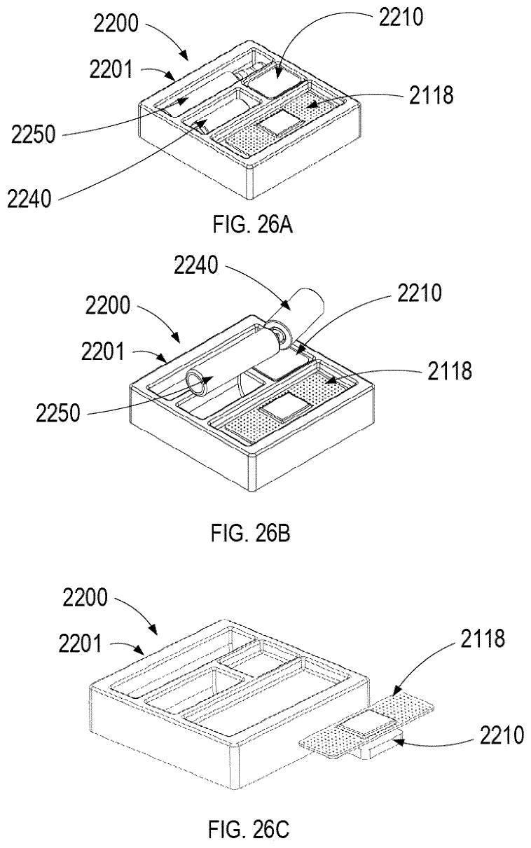

FIGS. 26A-26C are schematic illustrations of a sequence of steps performed using a kit 2200. The kit 2200 can include any of the systems used according to any of the methods described herein. For example, the kit 2200 can include an applicator pad 2210 and a reservoir 2250 containing fluid. As shown in FIG. 26A, the kit 2200 can include a tray 2201 housing the applicator pad 2210 and the reservoir 2250. The fluid in the reservoir 2250 can include a medication and/or a medication activating agent such as saline. As shown in FIG. 26B, the fluid can be transferred from the reservoir 2250 to the applicator pad 2210 to transfer medication to the applicator pad 2210 and/or to combine with dry medication predisposed in the applicator pad 2210. The reservoir 2250 can be similar to any of the reservoirs described herein. Furthermore, the fluid can be transferred from the reservoir 2250 using any of the applicator devices described herein. As shown, for example, the kit 2200 can include a grip 2240 that can be disposed over at least a portion (e.g., a neck portion or a half portion) of the reservoir 2250 as shown in FIG. 26B and rotated away from a central axis of the reservoir 2250 such that a portion of the reservoir 2250 disposed within the grip 2240 is broken (e.g., separated from a remainder of the reservoir 2250 outside of the grip 2240) and fluid is released.

As shown in FIG. 26C, a backing portion 2118 (e.g., a bandage) can be coupled to the applicator pad 2210 (e.g., via adhesive). The backing portion 2218 can include a convex distal surface configured to be coupled to the applicator pad 2210 to apply targeted and increased pressure to the wound via the applicator pad 2210. The applicator pad 2210 can then be disposed in contact with the wound with the backing portion 2218 being coupled to the skin surrounding the wound. In some embodiments, the backing portion 2218 can be removed after a predetermined period of time (e.g., four, five, six, or ten minutes).

In some embodiments, an applicator device such as any of the applicator devices described herein can be included in the kit 2200 can used to release the contents of the reservoir. The applicator device can then be used to apply the applicator pad 2210 to the wound and to apply pressure to the wound, or the applicator pad 2210 can optionally be separated from the applicator device (e.g., in the tray 2201) and the applicator pad 2210 can be applied to the wound and pressed against the wound using fingers and/or a palm of the subject or caregiver. Optionally, the backing portion 2218 can be applied to the applicator pad 2210 before or after applying the applicator pad 2210 to the wound.

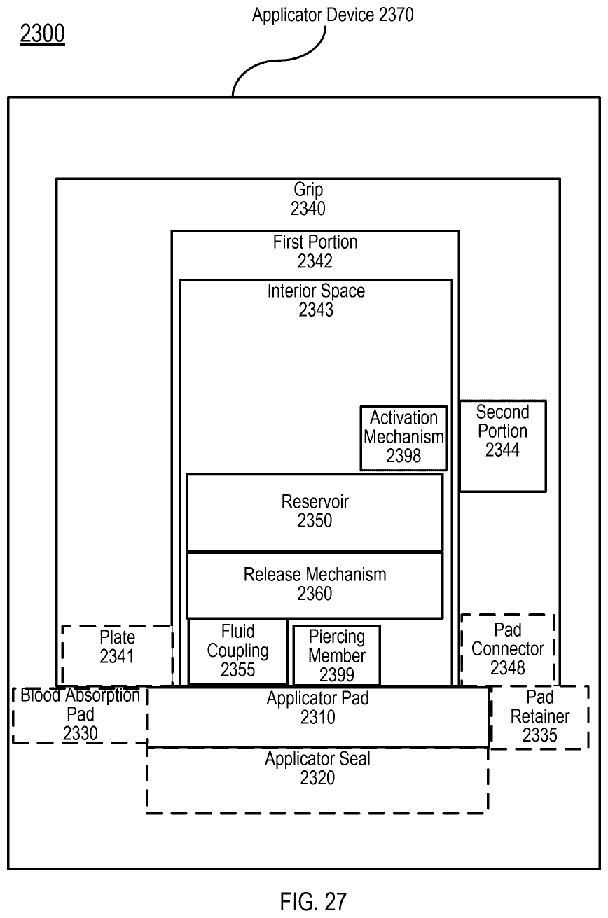

FIG. 27 is a schematic illustration of a bleeding treatment system 2300. Although the system 2300 is referred to as a bleeding treatment system and described with respect to treating a wound, the system 2300 (and any of the systems and devices described herein) can also be a system or device for topically applying any therapeutic or other substance to any suitable type of target treatment area that may benefit from application of the substance. The system 2300 can be the same or similar in structure and/or function to any of the bleeding treatment systems described herein. The system 2300 includes an applicator device 2370, an applicator pad 2310, and a reservoir 2350 including a release mechanism 2360. The applicator device 2370 includes a grip 2340 having a first portion 2342 defining an interior space 2343 configured to receive the reservoir 2350 and a second portion 2344 configured to initiate transfer of liquid from the reservoir 2350 to the applicator pad 2310 (e.g., via engagement or disengagement with an activation mechanism 2398, the reservoir 2350, the first portion 2343, and/or an intermediate component). The applicator device 2370 includes a piercing member 2399 configured to pierce the release mechanism 2360 to release liquid from the reservoir 2350 such that the liquid can flow from the reservoir 2350 to the applicator pad 2310 via a fluid coupling 2355 included in or defined by the grip 2340. The applicator device 2370 includes the activation mechanism 2398 described above, which is configured to urge liquid from the reservoir 2350 (e.g., upon being released via an engagement or disengagement with the second portion 2344 or in response to being urged by the second portion 2344).

In some embodiments, the reservoir 2350 can be configured such that liquid (e.g., medication such as TXA and/or activating liquid such as saline) within the reservoir 2350 can be displaced and/or pressurized to initiate flow of the liquid from the reservoir 2350 to the applicator pad 2310. For example, the reservoir 2350 can be closed on a distal end by the release mechanism 2360 that may be the same or similar in structure and/or function to any of the release mechanisms described herein. The release mechanism 2360 can be formed as a pierceable or breakable membrane, such as a rubber membrane (e.g., a resealable membrane) or a frangible membrane such as film or foil, that can be pierced by the piercing member 2399 to allow fluid to flow from the reservoir 2350. The reservoir 2350 can be configured to be moved (e.g., translated) into piercing contact with the piercing member 2399 such that fluid can be released and/or urged from the reservoir 2350. The reservoir 2350 can include a rigid housing at least partially defining an interior space within which the liquid is disposed. In some embodiments, the reservoir 2350 can be formed as a cartridge including a housing having a distal open end closed by a pierceable release mechanism 2360 (e.g., a cap, lined cap, or cover) in an initial configuration and a proximal open end closed by a movable piston or plunger. In some embodiments, the reservoir 2350 can be formed as a vial including a housing having a distal open end closed by a pierceable release mechanism 2360 (e.g., a cap, lined cap, or cover) in an initial configuration and a closed proximal end. In some embodiments, the reservoir 2350 can be formed as a syringe including a housing having a distal open end closed by a pierceable release mechanism 2360 (e.g., a cap, lined cap, or cover) in an initial configuration and a proximal end configured to receive a plunger therethrough such that a distal piston-end of the plunger is translatable within the housing and creates a fluidic seal with inner sidewalls of the housing to prevent liquid within an interior of the housing from flowing proximally beyond the piston-end of the plunger. In some embodiments, the reservoir 2350 (e.g., formed as a vial) can be preloaded with compressed gas (e.g., compressed argon gas) in addition to the liquid such that piercing or otherwise breaking the release mechanism 2360 results in the liquid being automatically urged from the reservoir 2350 under the force of the expanding gas. In some embodiments, the reservoir 2350 can be shaped and sized to hold and dispense 10 ml of fluid. In some embodiments, the reservoir 2350 can be shaped and sized to hold and dispense any suitable volume of fluid in an initial configuration, such as, for example, 3 ml of fluid, 5 ml of fluid, 7 ml of fluid, or 7.5 ml of fluid.

In some embodiments, the applicator device 2370 in combination with the reservoir 2350, or any of the applicator devices in combination with any of the reservoirs described herein, can be configured to contain liquid. In some embodiments, the applicator device 2370 in combination with the reservoir 2350, or any of the applicator devices in combination with any of the reservoirs described herein, can be configured to contain any suitable substance, such as any suitable flowable substance or any suitable higher viscosity substance (e.g., medication). For example, the applicator device 2370 in combination with the reservoir 2350, or any of the applicator devices in combination with any of the reservoirs described herein, can be configured to contain an ointment, a cream, a liniment, a paste, a lotion, a gel, a hydrogel, a liposome, a spray, an aerosol, a solution, a salve, or a higher viscosity medication, such as any substance described herein.

In some embodiments, rather than including a piercing member 2499, the release mechanism 2360 can be configured to burst as a result of a pressure of the liquid within the reservoir increasing above a threshold pressure. For example, the release mechanism 2360 can include a pressure seal, a silicone flapper seal, or a breakable adhesive. The liquid could then flow to the applicator pad 2310 via an opening in a distal end of the first portion 2342 and/or via the fluid coupling 2355.

The activation mechanism 2398 can include any suitable activation mechanism 2398 configured to urge the reservoir 2350 and/or the liquid within the reservoir 2350 (e.g., via an interaction with a piston and/or plunger of the reservoir 2350) toward the applicator pad 2310. In some embodiments, the activation mechanism 2398 can include a spring, a plunger configured to move a piston of the reservoir 2350, a plunger assembly configured to push air into the reservoir 2350 to displace liquid from the reservoir 2350, compressed gas pre-loaded into the reservoir 2350 with the liquid as described above, an activation reservoir configured to expand in response to a chemical reaction to propel the reservoir 2350 and/or liquid within the reservoir 2350 distally, and/or any other suitable activation mechanism.

The second portion 2344 of the grip 2340 can be formed as any suitable component configured to be manipulated (e.g., by a self-administering home user, a caregiver, or a patient) to release and/or cause movement of the activation mechanism 2398. For example, the second portion 2344 can be formed as a retaining pin configured to be engaged with the first portion 2342, the reservoir 2350, and/or the activation mechanism 2398 to prevent expansion of a spring of an activation mechanism 2398 from an initial configuration to an expanded configuration. In some embodiments, the retaining pin can be configured to be disengaged or decoupled from the spring (e.g., via being decoupled from a retaining member coupled to a distal end of the spring and/or from a piston of the reservoir 1250) such that the spring can expand to apply pressure to the reservoir 2350 (e.g., to urge the reservoir 2350 distally such that the release mechanism 2360 is pierced by the piercing member 2399 and/or to urge a piston and/or plunger of the reservoir 2350 distally to push liquid from the reservoir 2350). In some embodiments, the second portion 2344 can be formed as a plunger advanceable by a user relative to the first portion 2342 (e.g., while in contact with the reservoir 2350 to advance the reservoir 2350 itself and/or a plunger of the reservoir 2350) to apply pressure to the reservoir 2350. In some embodiments, the second portion 2344 can include threads configured to mate with threads of the first portion 2342 and can be configured to be rotated relative to the first portion 2342 such that the second portion 2344 is moved distally and applies pressure to the reservoir 2350 to urge the reservoir 2350 distally and/or to urge a piston and/or plunger of the reservoir 2350 distally to push liquid from the reservoir 2350. In some embodiments, the second portion 2344 can be formed as a distal housing portion configured to receive the applicator pad 2310 within an open distal end and configured to be translated relative to the first portion 2342 (e.g., into an interior of the first portion 2342) by applying a force to the first portion 2342 that is in the direction of a wound in contact with the applicator pad 2310.

In some embodiments, the fluid coupling 2355 can be a portion of the interior space 2343 defined by the housing of the first portion 2342 via which liquid can flow from the reservoir 2350 to the applicator pad 2310. In some embodiments, the fluid coupling 2355 includes a separate tubular member defining a lumen through which liquid can flow from the reservoir 2350 to the applicator pad 2310. In some embodiments, the fluid coupling 2355 can be a distal opening formed in the grip 2340. In some embodiments, the fluid coupling 2355 can be formed as a tubular portion coupled to the applicator pad 2310 configured to be coupled to the grip 2340 and/or a portion of the reservoir 2350 projecting distally from the grip 2340. For example, the fluid coupling 2355 can include a connector such as a Luer connector or a friction fit connector configured to be coupled to the grip 2340 and/or the reservoir 2350 such that fluid flowing from the reservoir 2350 can travel to the applicator pad 2310 through the fluid coupling 2355. After liquid transfer, the fluid coupling 2355 can be decoupled from the grip 2340 and/or the reservoir 2350, leaving the applicator pad 2310 in place (e.g., on a surface of a patient). In some embodiments, the fluid coupling 2355 can be a lumen defined by the piercing member 2399 (e.g., the piercing member 2399 can be a hollow needle).

In some embodiments, the piercing member 2399 can be disposed at a distal end of the grip 2340 (coupled to or included in the first portion 2342) and can project into the interior space 2343 toward the reservoir 2350. In some embodiments, the piercing member 2399 can be coupled to the applicator pad 2310 (e.g., disposed within the fluid coupling 2355 or within or adjacent another fluid coupling or connector), which may be couplable to and/or separable from the grip 2340. In some embodiments, the piercing member 2399 can be coupled to the second portion 2344. In some embodiments, the piercing member 2399 can include one or more lumens configured to allow the flow of liquid from the reservoir 2350 to the applicator pad 2310. In some embodiments, the piercing member 2399 or an additional piercing member can include one or more lumens configured to allow the flow of fluid (e.g., gaseous fluid) into the reservoir 2350 to displace liquid from the reservoir 2350. In some embodiments, the piercing member 2399 does not define a lumen. In some embodiments, the activation mechanism 2398 is configured to urge the release mechanism 2360 of the reservoir 2350 toward the piercing member 2399 such that the piercing member 2399 penetrates the release mechanism 2360 to initiate transfer of liquid from the reservoir 2350. In some embodiments, the first portion 2342 is configured to be translated distally relative to the piercing member 2399 and the reservoir 2350 can be in a fixed location relative to the first portion 2342 such that the release mechanism 2360 is translated into piercing contact with the piercing member 2399 to initiate transfer of liquid from the reservoir 2350 and the activation mechanism 2398 is configured to urge a portion of the reservoir 2350 (e.g., a piston) toward the piercing member 2399 to urge fluid from the reservoir 2350. The piercing member 2399 can be formed of any suitable material, such as metal or plastic.

In some embodiments, the grip 2340 is arranged relative to the applicator pad 2310 such that a central axis of the grip 2340 (or of the first portion 2342 of the grip 2340) is perpendicular to the wound-facing surface of the applicator pad 2310 (e.g., when the applicator pad 2310 is in contact with the wound). In some embodiments, the grip 2340 is arranged relative to the applicator pad 2310 such that a central axis of the grip 2340 (or of the first portion 2342 of the grip 2340) is disposed at an angle (e.g., a 45 degree angle) to the wound-facing surface of the applicator pad 2310 (e.g., when the applicator pad 2310 is in contact with the wound).

In some embodiments, rather than including one reservoir 2350 and one piercing member 2399, the system 2300 can include any suitable number of reservoirs and piercing members. For example, the system 2300 can include two reservoirs 2350 and two piercing members 2399, three reservoirs 2350 and three piercing members 2399, four reservoirs 2350 and four piercing members 2399, etc. In some embodiments, the system 2300 can include an activation mechanism 2398 (e.g., a spring) associated with each reservoir 2350, and a single second portion 2344 configured to simultaneously activate each of the activation mechanisms 2398. In some embodiments, the system 2300 includes one common activation mechanism 2398 configured to be activated by the second portion 2344.

The first portion 2342 can be formed in any suitable shape. For example, the first portion 2342 can include a cylindrical or tubular housing. In some embodiments, the first portion 2342 can include or be coupled to a plate 2341 disposed at the distal end of the first portion 2342 and configured to apply pressure to the applicator pad 2310 and thus a target wound. In some embodiments, the plate 2341 may form a bottom surface of the grip 2340. In some embodiments, the plate 2341 may be formed as a flange (e.g., a circumferential flange) extending from a bottom edge of the first portion 2342. In some embodiments, the plate 2341 can be separable from the grip 2340 (e.g., via decoupling the fluid coupling 2355 when the fluid coupling 2355 is coupled to the applicator pad 2310 or via decoupling the plate 2341 from the first portion 2342).

In some embodiments, the grip 2340 and/or applicator pad 2310 includes an applicator seal 2320 configured to cover at least the distal or skin-contacting surface of the applicator pad 2310 to prevent contamination of the applicator pad 2310 and, optionally, leakage from or drying of the applicator pad 2310 prior to use. The applicator seal 2320 can be formed, for example, as a film (e.g., a peelable film). In some embodiments, the applicator seal 2320 can be formed of a shrink wrap material covering the distal end of the applicator pad 2310 and positioned over at least a portion of the length of the first portion 2342.

The applicator pad 2310 can be the same or similar in structure and/or function to any of the applicator pads described herein. For example, the applicator pad 2310 can include and/or be coupled to an optional blood absorption pad 2330 and/or an optional pad retainer 2335. The blood absorption pad 2330 and the pad retainer 2335 can be the same or similar in structure and/or function to any of the blood absorption pads or pad retainers, respectively, described herein. In some embodiments, the applicator pad 2310 can have a cylindrical or conical shape.

In some embodiments, the applicator pad 2310 can be coupled to a backing portion (e.g., a bandage or a flexible or rigid cup) (e.g., via adhesive). The backing portion can be the same or similar in structure and/or function to any of the backing portions described herein, and can be configured to be coupled to the applicator pad 2310 to apply increased pressure to the wound via the applicator pad 2310 and to retain the applicator pad 2310 in place relative to the wound. The applicator pad 2310 can then be disposed in contact with the wound with the backing portion being coupled to the skin surrounding the wound. In some embodiments, the backing portion can be removed after a predetermined period of time (e.g., four, five, six, or ten minutes, 8 hours). In some embodiments, the backing portion can be applied to the applicator pad 2310 before or after applying the applicator pad 2310 to the wound. In some embodiments, the backing portion is coupled to the applicator pad 2310 before the applicator pad 2310 is wetted with the liquid from the reservoir 2350. Thus, in some embodiments, the backing portion can extend laterally from the grip 2340 or be folded upward against an outer surface of the grip 2340 when the applicator pad 2310 is coupled to the grip 2340 to receive the liquid form the reservoir 2350. In some embodiments, the backing portion can be graspable by a user and coupled to the applicator pad 2310 such that the applicator pad 2310 can be separated from the grip 2340 via pulling on the backing portion.

Prior to use, the applicator device 2370 can be disposed in an initial configuration. In some embodiments, in the initial configuration, the applicator pad 2310 can be coupled to the distal end of the grip 2340. The applicator seal 2320 can cover the applicator pad 2310. To use the applicator device 2370, the applicator seal 2320 can be optionally removed and the grip 2340 can be used to apply the applicator pad 2310 to a wound. To release the liquid from the reservoir 2350 such that the liquid flows to the applicator pad 2310, the second portion 2344 can be configured to be moved relative to the first portion 2342 (e.g., via translation relative to the first portion 2342, disengagement from the first portion 2342, and/or rotational movement relative to the first portion 2342) to engage or disengage with the reservoir 2350 and/or the release mechanism 2360.

The applicator pad 2310 can optionally be pressed against target wound tissue using the grip 2340. The applicator pad 2310 can optionally be maintained relative to the wound (e.g., without maintaining the grip 2340 against the applicator pad 2310) via coupling the applicator pad 2310 to the skin via the pad retainer 2335. The applicator pad 2310 can be optionally separated from the applicator pad 2310 via releasing the applicator pad 2310 from the grip 2340 (e.g., via releasing a pad connector 2348 optionally included in the applicator device 2370 which can be the same or similar in structure and/or function to any of the pad connectors described herein). In some embodiments, the applicator pad 2310 can be configured to separate from the first portion 2342 when saturated without manual intervention or without significant manual intervention (e.g., via expanding out of a recess the applicator pad 2310 is at least partially disposed within, due to an increased weight that causes the applicator pad 2310 to fall from a recess when wet, and/or due to the decreased friction between the applicator pad and the first portion 2342 when the applicator pad is saturated).

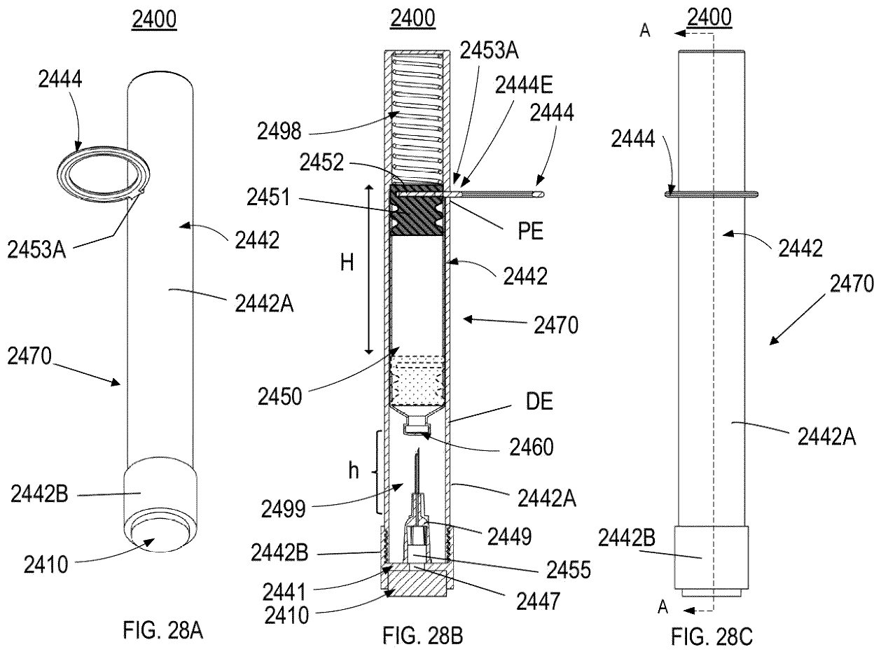

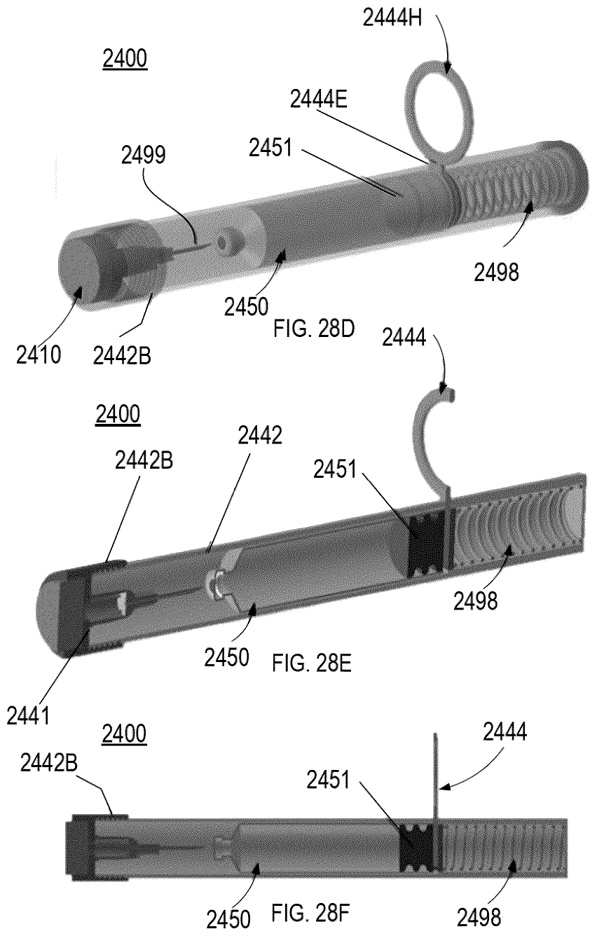

FIGS. 28A-28F show various views of an embodiment of a bleeding treatment system 2400. FIGS. 28A and 28D are isometric views of the bleeding treatment system 2400, FIGS. 28B and 28E-28F are cross-sectional views of the bleeding treatment system 2400 (taken along line A-A in FIG. 28C), and FIG. 28C is a side view of the bleeding treatment system 2400. The bleeding treatment system 2400 can be the same or similar in structure and/or function to any of the bleeding treatment systems described herein. As shown in FIG. 28B, the bleeding treatment system 2400 includes an applicator device 2470, an applicator pad 2410, and a reservoir 2450 including a release mechanism 2460. The applicator device 2470 includes a first portion 2442 defining an interior space configured to receive the reservoir 2450 and a second portion 2444 engaged with the first portion 2442 in an initial configuration to prevent the initiation of the transfer of liquid from the reservoir 2450 to the applicator pad 2410. The first portion 2442 can be formed in any suitable shape. For example, the first portion 2442 can include a cylindrical or tubular housing 2442A, as shown in FIGS. 28A-28F. The first portion 2442 includes a plate 2441 disposed at the distal end of the first portion 2442 and configured to apply pressure to an applicator pad 2410 and thus a target wound. In some embodiments, the plate 2441 can be included in a distal end portion 2442B separable from the housing 2442A of the first portion 2442 (e.g., may be attached to the first portion after the reservoir 2450 is placed within the first portion 2442 and/or may be detached from the first portion 2442 after transfer of liquid from the reservoir 2450 to the applicator pad 2410). As shown in FIG. 28B, the distal end portion 2442B can define a receptacle configured to receive at least a portion of the applicator pad 2410. The first portion 2442 also includes a piercing member 2499, which can project from the plate 2441 into the interior space toward the reservoir 2450.

Similar to the embodiment of the bleeding treatment system 2300, the bleeding treatment system 2400 may include an applicator seal (not shown) (similar to the applicator seal 2320 of the bleeding treatment system 2300) configured to cover at least the distal or skin-contacting surface of the applicator pad 2410 to prevent contamination of the applicator pad 2410 and, optionally, leakage from or drying of the applicator pad 2410 prior to use. The applicator seal can be formed, for example, as a film (e.g., a peelable film) such as a shrink wrap material. The applicator pad 2410 can be the same or similar in structure and/or function to any of the applicator pads described herein.

The reservoir 2450 can be formed as a cartridge including a rigid housing defining an interior configured to be prefilled with a medication (e.g., liquid). The reservoir 2450 can have a distal open end closed by the release mechanism 2460 (e.g., a cap or cover) and a proximal open end closed by a movable piston 2451. The release mechanism 2460 can be the same or similar in structure and/or function to any of the release mechanisms described herein (e.g., the release mechanism 2360). In the initial configuration of the system 2400, the movable piston 2451 is located at a proximal end PE of the housing of the reservoir 2450, as shown in FIG. 28B, and disposed at least partially within housing of the reservoir 2450. The movable piston 2451 is configured to be translated from the proximal end PE of the reservoir 2450 to a distal end DE of the reservoir 2450 to urge the fluid from the reservoir 2450 when the release mechanism 2460 is pierced by the piercing member 2499 to allow flow from the reservoir 2450. The piercing member 2499 defines a lumen through which liquid can flow from the reservoir toward the applicator pad 2410.

The applicator device 2470 includes an activation mechanism 2498 configured as a spring and arranged to apply pressure to the piston 2451 to urge the piston 2451 distally. When the activation mechanism 2498 is released, the activation mechanism 2498 can transition from a compressed state to an expanded state. Initially, due to the liquid disposed within the reservoir 2450 being substantially incompressible, the expansion of the activation mechanism 2498 toward (e.g., against) the piston 2451 causes the entire reservoir 2450 to be urged towards the piercing member 2499 such that the piercing member 2499 pierces the release mechanism 2460 and releases liquid from the reservoir 2450. The liquid can flow from the reservoir 2450 to the applicator pad 2410 via the lumen of the piercing member 2499.

As shown in FIGS. 28A-28F, the second portion 2444 is formed as a retaining pin 2444. The retaining pin 2444 can include an extension member 2444E and a handle portion 2444H. Although the handle portion 2444H is shown as being ring-shaped, the handle portion 2444H can have any suitable shape. Additionally, although the handle portion 2444H is shown as extending perpendicularly away from a central axis of the first portion 2444, in some embodiments the handle portion 2444H can extend in any suitable direction relative to the extension member 2444E (e.g., parallel to the central axis, adjustable relative to the extension member 2444E about a hinged coupling). The first portion 2444 and the piston 2451 are configured to receive the extension member 2444E of the retaining pin 2444 in the initial configuration of the system such that the retaining pin 2444 retains the activation mechanism 2498 in the initial configuration and prevents movement of the activation mechanism 2498 and the piston 2451 relative to the first portion 2442. The first portion 2442 defines an opening 2453A in the housing 2442A configured to receive the extension member 2444E of the retaining pin 2444. In some embodiments, the housing 2442A defines a second opening or recess opposite the opening 2453A within which the free end of the extension member 2444E can be disposed in the initial, retaining configuration. In some embodiments, the extension member 2444E can be visually identifiable within the second opening (e.g., by the user) to confirm that the extension member 2444E is properly engaged with the first portion 2442 and the piston 2451. Additionally, the piston 2451 defines a through-hole or socket 2452 configured to receive the extension member 2444E such that the piston 2451 can be retained in place relative to the opening 2453A and the optional second opening by the extension member 2444E. In some embodiments, the piston 2451 can include a piston portion and a retention portion coupled to a proximal end of the piston portion, and the through-hole or socket 2452 can be defined between the piston portion and the retention portion. The piston 2451 (e.g., the retention portion) can include a flat proximal surface such that the activation mechanism 2498 can apply even pressure to the piston 2451.

The reservoir 2450 can be retained in a position in which the release mechanism 2460 is spaced from the piercing member 2499 by the engagement between the extension member 2444E and the through-hole 2452. Removal of the retaining pin 2444 from the through-hole 2452 and the opening 2453A allows for the reservoir 2450 to be translated distally by the activation mechanism 2498 such that the activation mechanism is thereby released. The activation mechanism 2498 can then push the reservoir 2450 into sufficient engagement with the piercing member 2499 such that the tip of the piercing member 2499 pierces the release mechanism 2460. Once the release mechanism 2460 is pierced, the activation mechanism 2498 can push the piston 2451 from the proximal end PE towards the distal end DE of the reservoir 2450, causing the delivery of the fluid from the reservoir 2450 via the lumen of the piercing member 2499.

In some embodiments, an inner diameter of the first portion 2442 in a distal region of the first portion 2442 (in which the housing of the reservoir 2450 is disposed in the initial configuration of the system 2400) can be larger than an inner diameter of the first portion 2442 in a proximal region of the first portion 2442 (in which the activation mechanism 2498 is disposed in the initial configuration of the system 2400) such that the piston 2451 and activation mechanism 2498 do not stick during expansion of the activation mechanism 2498. The piston 2451 can, in some embodiments, be partially disposed between the distal region and the proximal region of the first portion 2442. The distal region and the proximal region of the first portion 2442 can be separated, for example, by the first opening 2453A and the second opening. In some embodiments, the inner diameter of the housing of the cartridge can be the same or larger than the inner diameter of the proximal region of the first portion 2442.

In some implementations, rather than the retaining pin 2444 engaging with the piston 2451 to prevent release of the activation mechanism 2498 by preventing translation of the piston 2451, the retaining pin 2444 can be engaged directly with the activation mechanism 2498 or another component coupled to a distal end of the activation mechanism to prevent expansion of the activation mechanism 2498.

In the example implementation, the activation mechanism 2498 may be a coiled spring under compression, as shown, for example, in FIG. 28B. In various implementations, the parameters of the coiled spring (e.g., elasticity of the coil, a thickness of the coil, a number of turns for the coil, a diameter for a turn of the coil, and the like) are selected such that the force produced by the activation mechanism 2498 onto the proximal end PE of the reservoir 2450 is sufficient to urge the release mechanism 2460 into contact with the piercing member 2460 to cause rupturing of the release mechanism 2460, and to push the movable piston 2451 towards the distal end DE of the reservoir 2450. Further, in some implementations, the coiled spring forming the activation mechanism 2498 is configured to expand a distance H+h (as shown in FIG. 28B) to an equilibrium position (e.g., the position in which it applies no pressure on the movable piston 2451), such that the movable piston 2451 substantially reaches the distal end DE of the reservoir 2450 (a dashed contour of the movable piston is shown in FIG. 28B at the distal end DE of the reservoir as merely an indication where the movable piston will be when pushed all the way to the distal end DE). Note that distance H is the distance traveled by the movable piston 2451 and distance h is the distance traveled by the reservoir 2450 after it is being pushed by the activation mechanism 2498. In some cases, even after expanding the distance H+h, the coiled spring may not reach the equilibrium position (thereby continuing to apply pressure onto the movable piston 2451 when the movable piston 2451 is at the distal end DE of the reservoir 2450).

As described above and shown in FIG. 28B, the piercing member 2499 is disposed on the plate 2441 opposite the applicator pad 2410. The piercing member 2499 can include or be coupled to a hub 2449 that is coupled to the plate 2441 and has an interior in fluid communication with the lumen of the piercing member 2499. The plate 2441 defines an opening 2447 through which the fluid from the reservoir 2450 can flow toward the applicator pad 2410. Thus, the hub 2449 and the opening 2447 can individually or collectively form a fluid coupling 2455. Additionally, or alternatively, in some implementations, the fluid coupling may be achieved by the fluid flowing outside the piercing member 2499. For instance, the fluid may flow outside member 2499, through one or more openings in the plate 2441, and to the applicator pad 2410.

To use the applicator device 2470, the optional applicator seal can be removed and the applicator device 2470 can be placed such that the applicator pad 2410 contacts a wound on a surface of a skin of the user. For example, if the user has a wound on one arm, the user can use one hand of the opposite arm to dispose the applicator device 2470 over the wound such that the applicator pad 2410 is in contact with the wound. The user can pull the retaining pin 2444 with a finger of the one hand (i.e., the hand that holds the applicator device 2470), thereby releasing the activation mechanism 2498 to initiate release of liquid from the reservoir 2450 to the absorbent pad 2410 and, thus, to the wound. The user can maintain pressure against the wound by holding the applicator device 2470 against the absorbent pad 2410 against the wound as described herein. Alternatively, the user can pull out the retaining pin 2444 prior to using the applicator device 2470, and, while the applicator pad 2410 is being saturated with the fluid from the reservoir 2450 or after the saturation operation is complete, the user can dispose the applicator pad 2410 against the wound and maintain the applicator device 2470 against the applicator pad 2410 to apply compression to the wound via the plate 2441 and the applicator pad 2410.

In some cases, as described above, the applicator pad 2410 saturated with the fluid from the fluid reservoir 2450 may be releasable from the first portion 2442 and may optionally be configured to remain coupled to the wound via being attached to skin surrounding the wound (via, for example, adhesive) for continued compression of the wound (e.g., via the user's hand against the applicator pad 2410 and/or compression applied by the applicator pad 2410 itself). Additionally, the applicator pad 2410 can be used to prevent contamination of the wound and for continued wound treatment with a medication. In some implementation, the applicator pad 2410 may be coupled to the first portion 2442 by means of a sticky adhesive layer, and may be detachable by separating (e.g., manually) the applicator pad 2410 from the first portion 2442 (e.g., after the applicator pad 2410 is coupled to the wound of the user). Alternatively, the applicator pad 2410 may be coupled to the first portion 2442 by means of a pad retainer, such as any of the pad retainers described herein. For example, the pad retainer may include one or more hooks, clamps, and the like. In some embodiments, the applicator pad 2310 may be disposed within the receptacle defined by the distal end portion 2442B via a compression or friction fit, and may expand and separate from the distal end portion 2442B upon being wet with the liquid from the reservoir 2450.

In some implementations, the applicator pad 2410 may not be decoupled from the first portion 2442 and may be configured to be held against the wound for a duration of time required for the wound treatment (e.g., the applicator pad 2410 may be held against the wound for a minute, a few minutes, ten minutes, a few tens of minutes, a few hours, and the like, for facilitating coagulation of blood in the wound). The applicator pad 2410 may be pressed against the wound by user pressing the applicator device 2470 towards the wound. The applicator pad may be left on the wound with an adhesive bandage for hours (e.g., up to 24 hours).

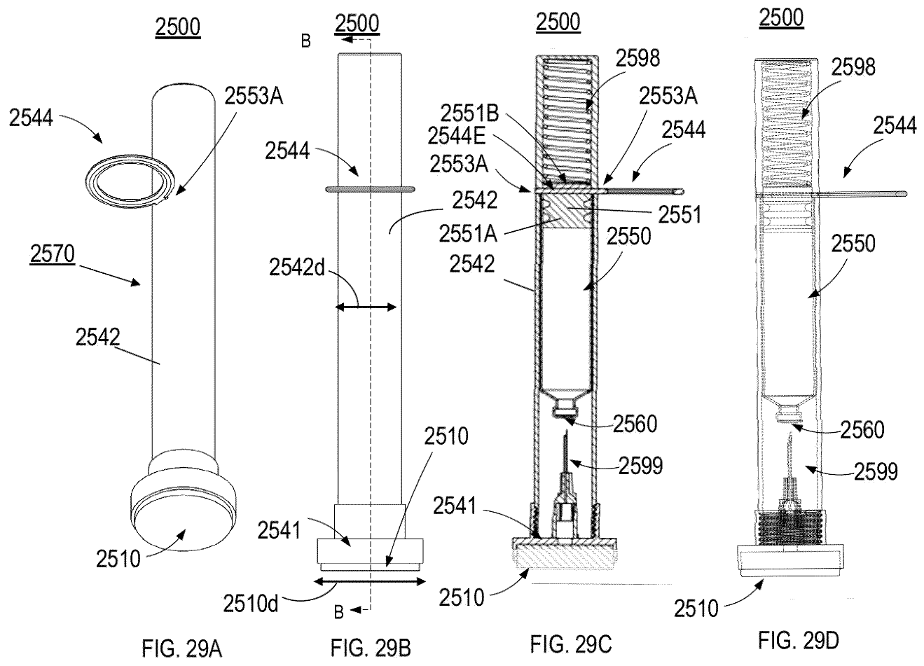

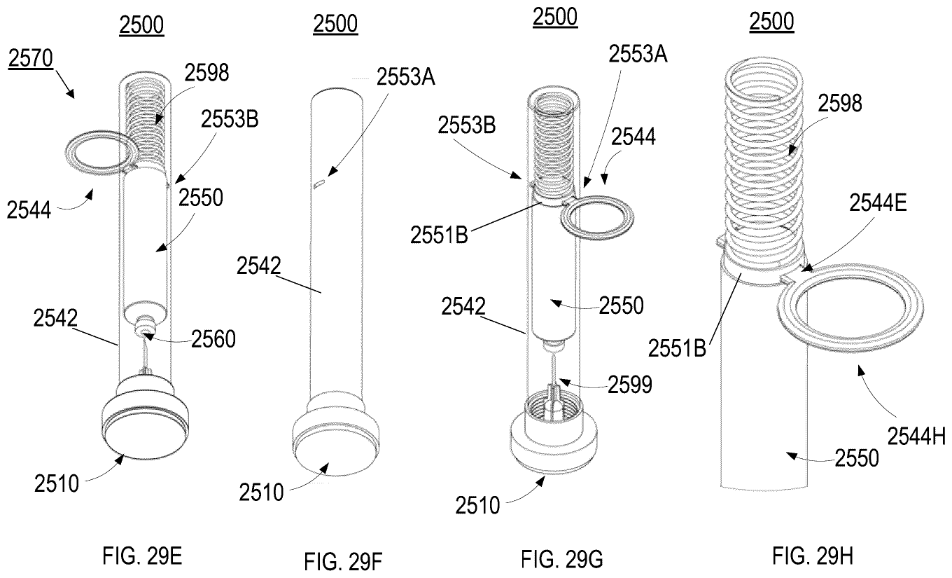

The applicator pads described herein, such as the applicator pad 2410, can be any suitable shape and size. For example, FIGS. 29A-29H show an example embodiment of a bleeding treatment system 2500 including an applicator device 2570 that may be similar to the applicator device 2470 of the bleeding treatment system 2400, but with the difference that the diameter 2510d of an applicator pad 2510, as shown in FIG. 29B, is larger than the diameter 2542d of a first portion 2542. FIG. 29A is a perspective view, FIG. 29B is a side view, FIG. 29C is a cross-sectional view taken along line B-B in FIG. 29B, FIG. 29D is a section view, FIG. 29E is a perspective view with the first portion 2542 shown as transparent, FIG. 29F is a perspective view with the second portion 2544 uncoupled from the first portion 2542, FIG. 29G is a perspective view with the first portion 2542 shown as transparent, and FIG. 29H is a perspective view of a portion of the system 2500 with the first portion 2542 not shown. Like in the bleeding treatment system 2400 described above, the applicator device 2570 can include a plate 2541, a second portion 2544, and an activation mechanism 2598, and can be configured to receive a reservoir 2550 including a piston 2551 and coupled to a release mechanism 2560. In some embodiments, the applicator pad 2510 can be any suitable size, such as dimensioned to be at least 3.5 cm wide in at least one direction to be able cover a wound that is about 3 cm wide.

The first portion 2542 and the second portion 2544 can be the same or similar in structure and/or function to the first portion 2442 and the second portion 2444 described above. For example, the second portion 2544 can be formed as a retaining pin 2544. The retaining pin 2544 can include an extension member 2544E and a handle portion 2544H. The first portion 2544 and the piston 2551 are configured to receive the extension member 2544E of the retaining pin 2544 in the initial configuration of the system such that the retaining pin 2544 retains the activation mechanism 2598 in the initial configuration and prevents movement of the activation mechanism 2598 and the piston 2551 relative to the first portion 2542. The first portion 2542 defines a first opening 2553A and a second opening 2553B on opposite sides of the housing 2542. Each of the first opening 2553A and the second opening 2553B are configured to receive the extension member 2544E of the retaining pin 2544 in the initial, retaining configuration. In some embodiments, the free end of the extension member 2544E can be visually identifiable within the second opening 2553B (e.g., by the user) to confirm that the extension member 2544E is properly engaged with the first portion 2542 and the piston 2551. Additionally, the piston 2551 defines a through-hole or socket 2552 configured to receive the extension member 2544E such that the piston 2551 can be retained in place relative to the openings 2553A, 2553B by the extension member 2544E. In some embodiments, the piston 2551 can include a piston portion 2551A and a retention portion 2551B coupled to a proximal end of the piston portion 2551, and the through-hole or socket 2552 can be defined between the piston portion 2551A and the retention portion 2551B. The piston 2551 (e.g., the retention portion 2551B) can include a flat proximal surface (as shown in FIG. 29H) such that the activation mechanism 2598 can apply even pressure to the piston 2551.

The reservoir 2550 can be retained in a position in which the release mechanism 2560 is spaced from the piercing member 2599 by the engagement between the extension member 2544E and the through-hole 2552. Removal of the retaining pin 2544 from the second opening 2443B, the through-hole 2552, and the first opening 2553A allows for the reservoir 2550 to be translated distally by the activation mechanism 2598 such that the activation mechanism is thereby released. The activation mechanism 2598 can then push the reservoir 2550 into sufficient engagement with the piercing member 2599 such that the tip of the piercing member 2599 pierces the release mechanism 2560. Once the release mechanism 2560 is pierced, the activation mechanism 2598 can push the piston 2551 from the proximal end PE towards the distal end DE of the reservoir 2550, causing the delivery of the fluid from the reservoir 2550 via the lumen of the piercing member 2599.

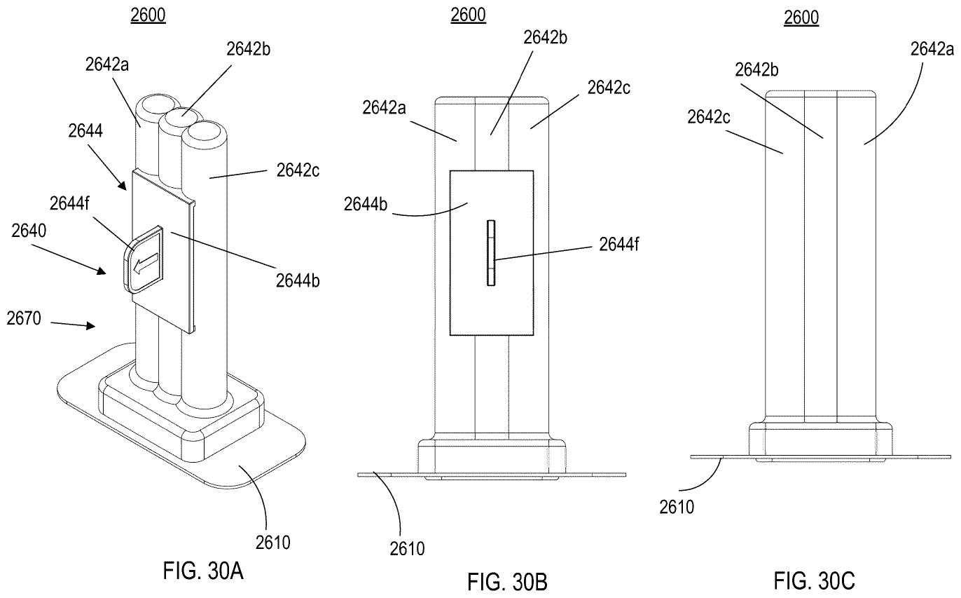

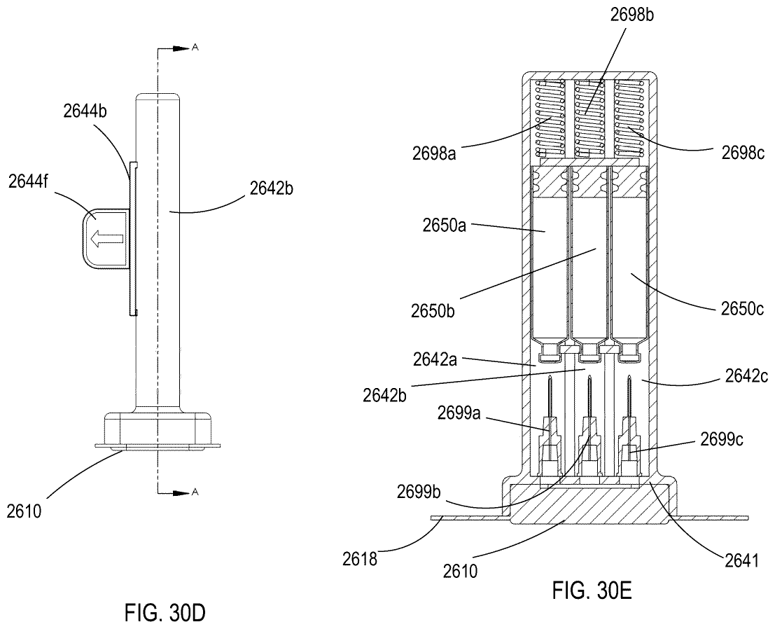

FIGS. 30A-30E show an example implementation of a bleeding treatment system 2600. FIG. 30A shows an isometric view of the bleeding treatment system 2600, FIG. 30B shows a front view of the bleeding treatment system 2600, FIG. 30C shows a back view of the bleeding treatment system 2600, FIG. 30D shows a side view of the bleeding treatment system 2600, and FIG. 30E shows a cross-sectional view of the bleeding treatment system 2600 taken along cross-section A-A shown in FIG. 30D. The bleeding treatment system 2600 can be the same or similar in structure and/or function to any of the bleeding treatment systems described herein, such as the bleeding treatment system 2400. For example, the bleeding treatment system 2600 includes the applicator device 2670. The bleeding treatment system 2600 also includes a plurality of reservoirs, such as reservoirs 2650a-2650c (each reservoir 2650a-2650c may be similar to the reservoir 2450, as shown in FIGS. 28A-28G) and an applicator pad 2610, which may be the same or similar in structure and/or function to and of the applicator pads described herein. The applicator device 2670 including a grip 2640 having a plurality of first portions, such as the first portions 2642a-2642c, (each first portion 2642a-2642c may be similar to the first portion 2442, as shown in FIGS. 28A-28F), a plurality of activation mechanisms, such as activation mechanisms 2698a-2698c (each activation mechanism 2698a-2698c may be similar to the activation mechanism 2498, as shown in FIGS. 28A-28G), a plurality of piercing members, such as piercing members 2699a-2699c (each piercing member 2699a-2699c may be similar to the piercing mechanism 2499, as shown in FIGS. 28A-28G), and fluid couplings (e.g., hubs, openings in plate 2641) between piercing mechanisms 2699a-2699c and the applicator pad 2610. Having multiple first portions, reservoirs, activation members, and piercing mechanisms may allow to readily scale the size of the bleeding treatment system 2600 for wounds of a selected size, and allow for dispensing a particular volume of liquid regardless of the availability of cartridges having a particular volume.

The activation mechanisms 2698a-2698c may be released at the same time when a user pulls out a retaining clip 2644 (the retaining clip 2644 is shown, for example, in FIG. 30A). The retaining clip 2644 may include a flap 2644f configured to be grabbed by a user and a body portion 2644b having extension elements which may be inserted into suitable sockets for preventing the release of activation mechanisms 2698a-2698c, and, in some implementations, for securing the reservoirs 2650a-2650c in place relative to the first portions 2642a-2642c. Removing the retaining clip 2644 from first portions 2642a-2642c releases activation mechanisms 2698a-2698c, thereby moving reservoirs 2650a-2650c towards associated piercing members 2699a-2699c and piercing release mechanisms (which, for example, may be similar to a release mechanism 2460, as shown in FIGS. 28A-28G) of reservoirs 2650a-2650c, and moving pistons within the reservoirs 2650a-2650c to urge fluid to flow from the reservoirs via fluid couplings (e.g., lumens and hubs) associated with the piercing mechanism 2699a-2699c towards the applicator pad 2610.

The applicator pad 2610 can be coupled to a backing portion 2618 that may be configured as a bandage. The backing portion 2618 can include an adhesive covered by a peel-away adhesive liner disposed on a distal surface of the backing portion 2618. The adhesive liner can be peeled away prior to placing the backing portion 2618 on a subject's skin so that the backing portion 2618 can be adhered to the skin via the adhesive. The backing portion 2618 may be similar or the same as any of the backing portion described herein.

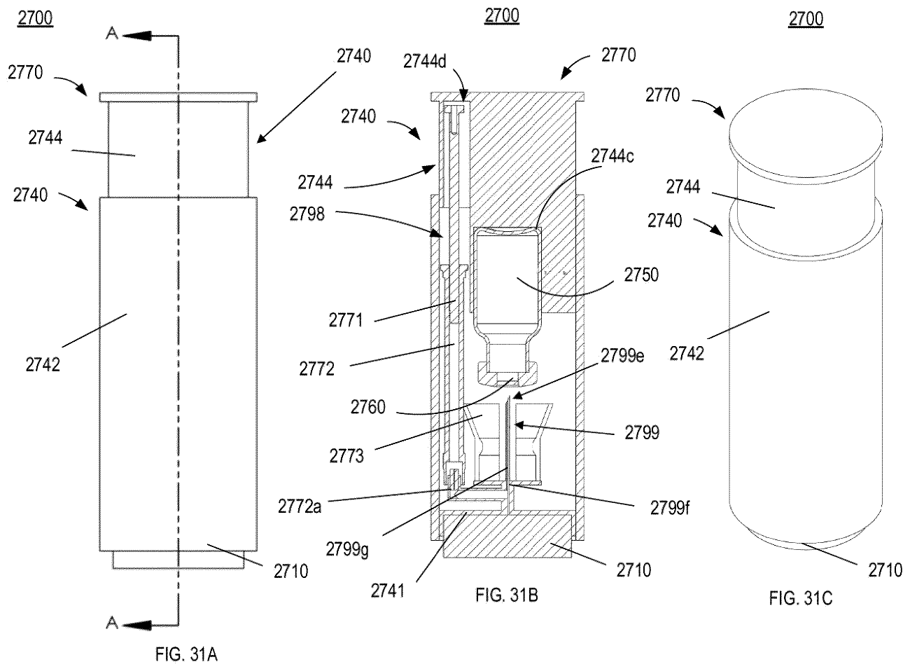

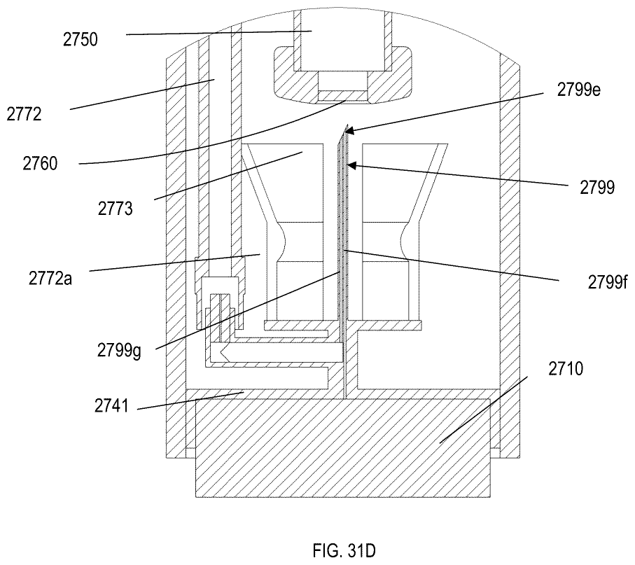

In some embodiments, rather than the reservoir being formed as a cartridge, the reservoir can be formed as a vial. For example, FIGS. 31A-31C show an embodiment of a bleeding treatment system 2700. FIG. 31A shows a side view of the treatment system 2700, FIG. 31B shows a cross-sectional view of the treatment system 2700 taken along line A-A section in FIG. 31A, and FIG. 31C shows a perspective view of the treatment system 2700. FIG. 31D is a cross-sectional view of a distal portion of the system 2700. The bleeding treatment system 2700 can be the same or similar in structure and/or function to any of the bleeding treatment systems described herein, such as the bleeding treatment system 2400. For example, the bleeding treatment system 2700 includes an applicator device 2770, an applicator pad 2710, and a reservoir 2750, which can be the same or similar in structure and/or function to any of the applicator devices, applicator pads, and reservoirs, respectively, described herein. As shown in FIG. 31B, the reservoir 2750 can be formed as a vial including a housing defining an interior configured to contain liquid and a release mechanism 2760 (e.g., a cap or cover) that is pierceable to access the contents of the reservoir 2750. The reservoir 2750 can include any suitable vial having any suitable volume. For example, the reservoir 2750 may be formed as a glass vial, a plastic vial, and the like. The volume can include, for example, 10 mL of fluid, or enough fluid such that 10 mL of fluid can be dispensed by the system 2700 (e.g., if some liquid remains in the reservoir 2750 after the applicator device 2870 is transitioned to a fully dispensed configuration).

The applicator device 2770 includes a grip 2740 having a first portion 2742 and a second portion 2744 configured to move (e.g., translate) relative to the first portion 2742. As shown in FIG. 31B, the second portion 2744 is formed as a piston configured to move (e.g., translate) within an interior space defined by the first portion 2742. The second portion 2744 defines a cavity 2744c which houses and retains a portion of the reservoir 2750 (e.g., via any suitable coupling mechanism such as a friction fit and/or adhesive). The applicator device 2770 includes a piercing member 2799 coupled to a distal end of the first portion 2722 and protruding distally such that the reservoir 2750 can be moved toward the piercing member 2799 and the piercing member 2799 can pierce the release mechanism 2760 of the reservoir 2750. As shown in FIG. 31B, the distal end of the first portion 2722 can be formed at least in part by a plate 2741 in contact with a proximal surface of the applicator pad 2710 and configured to apply compression to the applicator pad 2710 (and, thus, a wound in contact with the applicator pad 2710). The plate 2741 can have a continuous distal surface except for a fluid through-hole to allow liquid from the reservoir 2750 to flow to the applicator pad 2710. The piercing member 2799 can protrude distally from the plate 2741 and can define a lumen 2799f (also referred to as a fluid channel) in fluid communication with the opening of the plate 2741. The piercing member 2799 can also define a lumen 2799g (also referred to as a gas channel) fluidically isolated from the lumen 2799f. The lumen 2799g can be arranged in parallel to the lumen 2799f. In some embodiments, rather than including a single piercing member 2799 defining two lumens, the applicator device 2770 can include two piercing members 2799 disposed sufficiently close to each other to be able to both pierce the release mechanism 2760, with one of the piercing members 2799 defining the lumen 2799g and the other piercing member defining the lumen 2799f. Additionally, the applicator device 2770 includes a receiving hub 2773 configured to receive a distal end of the vial 2750 and stabilize the vial 2750 relative to the piercing member 2799 when the vial 2750 is translated distally. The receiving hub 2773 can include, for example, a set of tabs (e.g., four tabs) configured to flex relative to a base of the receiving hub 2773 such that the distal end of the vial 2750 can urge the tabs outward when the distal end of the vial 2750 is in contact with the receiving hub 2773 and the tabs can flex back into place to secure the distal end (e.g., the cap-shaped release mechanism 2360) in a distal portion of the receiving hub 2773 distally of protrusions of the tabs.

The applicator device 2770 also includes an activation mechanism 2798 (also referred to as an activation assembly) configured to initiate urging of liquid from the reservoir 2750. The activation mechanism 2708 includes a plunger 2771 and a barrel 2772 defining a gas reservoir. The gas reservoir of the barrel 2772 is fluidically coupled to the lumen 2799g of the pierceable member 2799 via a fluid coupling 2772a, which may include any suitable combination of fluid couplings such as tubular fluid connectors and may include any suitable filters or valves (e.g., one-way valves). The plunger is configured 2771 to be translated relative to the barrel 2772 to urge gas initially disposed within the barrel 2772 into the lumen 2799g via the fluid coupling 2772a. As shown in FIG. 31B, the second portion 2744 can define a plunger cavity 2744d configured to receive a portion of the plunger 2771 such that distal translation of the second portion 2744 causes the plunger 2771 to be advanced distally relative to the barrel 2772 due to contact between the proximal end of the plunger and the second portion 2774.

In use, a user can apply pressure to the second portion 2744 to advance the second portion 2744 distally relative to the first portion 2742. Due to the reservoir 2750 being retained with in the cavity 2744c and the plunger 2771 being disposed within the cavity 2744d, as the second portion 2744 moves distally towards the piercing member 2799, both the plunger 2771 and the reservoir 2750 are also moved distally under the control of the second portion 2744. Once the release mechanism 2760 is pierced by the piercing member 2799 and the proximal or free end of the piercing member 2799 (including the proximal ends of the lumen 2799g and the lumen 2799f) is disposed within the interior of the reservoir 2750 (due to the second portion 2744 advancing the reservoir 2750 sufficiently far), further distal translation of the second portion 2744 causes the plunger 2771 to apply pressure to gas within the barrel 2772, thereby expelling at least some amount of the gas from the barrel 2772, through the fluid coupling 2772a, through the lumen 2799g of the piercing member 2799, and into the interior of the reservoir 2750. The gaseous fluid that travels into the interior of the reservoir 2750 can displace the liquid fluid in the reservoir 2750 to urge the liquid fluid through the lumen 2799f to the applicator pad 2710.

In some embodiments, the lengths of the plunger 2771, the barrel 2772, and the piercing member 2799 and the diameter of the barrel 2772 can each be selected to optimize liquid fluid transfer from the vial 2750 to the absorbent pad 2710. For example, the lengths and diameter may be selected such that the piercing member 2799 is sufficiently short to be able to receive liquid from a bottom portion of the vial 2750, while the barrel 2772 may have a sufficiently large diameter such that sufficient gas can be introduced to the vial 2750 to urge the liquid from the vial 2750 even at shorter barrel and/or plunger lengths. In some embodiments, the proximal end of the plunger 2798 can be spaced from the contacting wall of the cavity 2744d such that the second portion 2744 does not begin to translate the plunger 2771 until the release mechanism 2760 is pierced by the piercing member 2799 or shortly thereafter. To avoid creation of a low-pressure region (e.g., a partial vacuum) within the interior of the reservoir 2750, components of the activation mechanism 2798 relative to the remainder of the applicator device 2770 and the reservoir 2750 can be configured to deliver a suitable volume of gas at a suitable flow rate to the interior of the reservoir 2750 to sufficiently displace liquid from the reservoir 2750 for fluid flow without negative pressure-induced interruption.

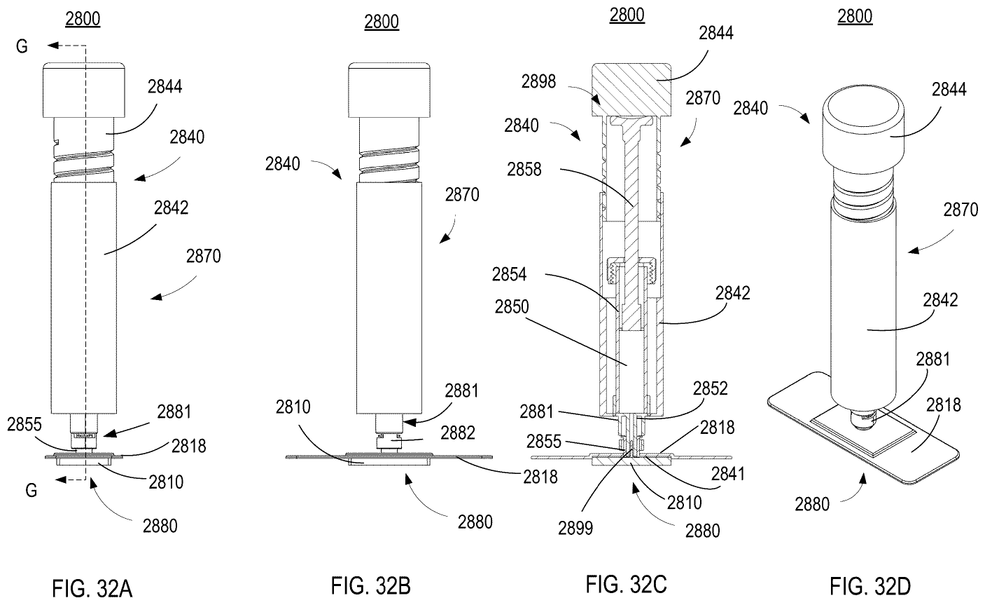

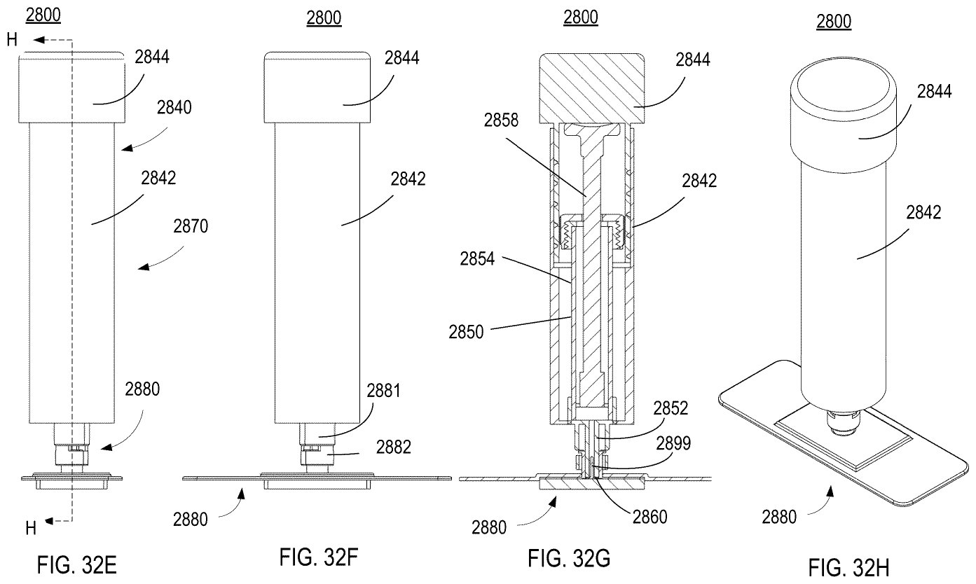

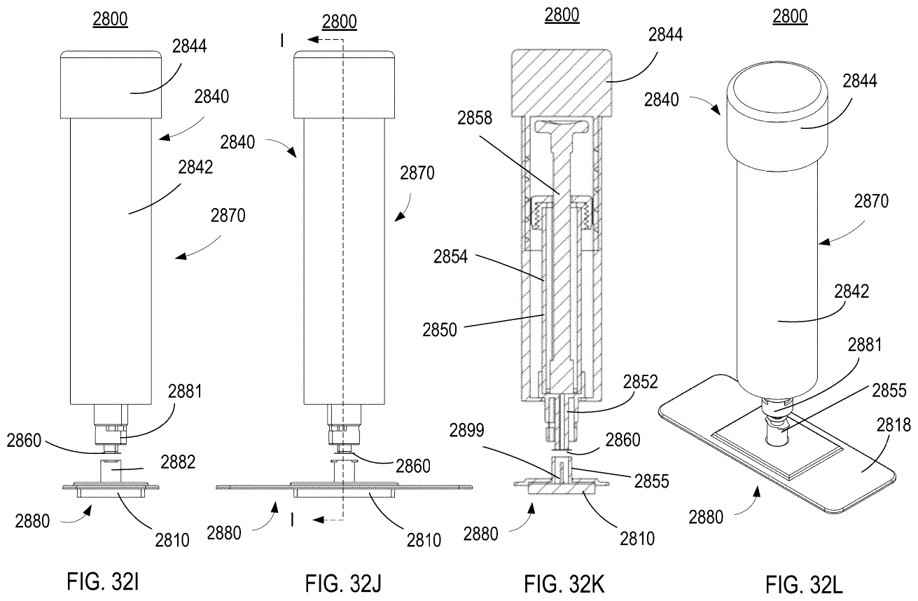

In some embodiments, rather than the reservoir being formed as a vial or a cartridge, the reservoir can be formed as a syringe. FIGS. 32A-32L show an embodiment of a bleeding treatment system 2800 in a partially dispensed configuration (FIGS. 32A-32D), a fully dispensed configuration (FIGS. 32E-32H), and an uncoupled configuration (FIGS. 32I-32L). FIGS. 32A, 32E, and 32I are front views of the bleeding treatment system 2800, FIGS. 32B, 32F, and 32J are side views of the bleeding treatment system 2800, and FIGS. 32C, 32G, and 32K are cross-sectional views of the bleeding treatment system 2800 taken along lines G-G in FIG. 32A, H-H in FIG. 32E, and I-I in FIG. 32J, respectively. FIGS. 32D, 32H, and 32L are perspective views of the bleeding treatment system 2800.

The bleeding treatment system 2800 can be the same or similar in structure and/or function to any of the bleeding treatment systems described herein, such as the bleeding treatment system 2400. For example, the bleeding treatment system 2800 includes an applicator device 2870, a pad assembly 2880 including an applicator pad 2810, and a reservoir 2850, which can be the same or similar in structure and/or function to any of the applicator devices, applicator pads, and reservoirs, respectively, described herein. As shown in FIG. 32C, the reservoir 2850 can be formed as a syringe including a plunger 2858, a barrel 2854 defining an interior configured to contain liquid, and a release mechanism 2860 (e.g., a cap or cover) that is pierceable to access or release the contents of the reservoir 2850. The reservoir 2850 can include any suitable barrel 2854 having any suitable volume. For example, the reservoir 2850 may be formed as a glass syringe, a plastic syringe, and the like. The volume can include, for example, 10 mL of fluid, or enough fluid such that 10 mL of fluid can be dispensed by the system 2800 (e.g., if some liquid remains in the reservoir 2850 such as in a neck 2852 of the reservoir 2850 after the plunger is transitioned to a fully dispensed configuration).

The applicator device 2870 includes a first portion 2842 and a second portion 2844. The first portion 2842 and the second portion 2488 may be the same or similar in structure and/or function to any of the first portions and the second portions described herein. The first portion 2842 and the second portion 2844 collectively form an interior space within which at least a portion of the reservoir 2850 can be disposed. As shown in FIG. 32C, the first portion 2842 can define a distal opening through which a portion of the reservoir 2850 can extend (e.g., the neck portion 2852). The barrel 2854 of the reservoir 2850 can have at least a portion that has a larger outer diameter than the distal opening of the first portion 2842 such that the barrel 2854 is prevented from moving distally relative to the first portion 2842, while the neck portion 2852 extends beyond the distal end of the first portion 2842. The barrel 2850 can be coupled to the first portion 2824 via any suitable coupling mechanism such that rotation of the first portion 2824 by the user causes corresponding rotation of the barrel 2850. For example, the barrel 2850 can be coupled via adhesive, friction fit, via mating interlocking features, or via projections or shapes of the barrel 2850 and/or the interior walls of the first portion 2824 that prevent rotation of the barrel 2850 relative to the first portion 2824.

The first portion 2842 and the second portion 2844 include mating threads such that the second portion 2844 can be twisted relative to the first portion 2842 to advance the second portion 2844 distally relative to the first portion 2842. Although not shown, in some embodiments the second portion 2844 can include features (e.g., laterally extending tabs) to assist a user in rotating the second portion 2844 relative to the first portion 2842. When the reservoir 2850 disposed in the interior space defined by the first portion 2842 and the second portion 2844, the advancement of the second portion 2844 relative to the first portion 2842 can cause an interior proximal surface the second portion 2844 to contact and/or urge the plunger 2858 of the reservoir 2850 distally. Thus, the plunger 2858 and/or the portion of the second portion 2844 configured to contact the plunger 2858 can function as an activation mechanism 2898 that is activatable by rotating the second portion 2844 relative to the first portion 2842. In some embodiments, rather than being coupled via mating threads, the second portion 2844 and the first portion 2842 can be coupled via any suitable mechanism(s) such that the second portion 2844 can be translated or rotated to advance the plunger 2858 relative to the barrel 2854. In some embodiments, the applicator device 2870 can include a spring similarly as described with respect to the applicator device 2470 and be configured such that rotation of the second portion 2844 or movement of another element (e.g., a latch, pin, and/or button) releases the spring such that the spring can apply a distal force to the plunger 2858.

The pad assembly 2880 includes a piercing member 2899. The pad assembly 2880 can also include a plate 2841 coupled to at least a portion of a proximal surface of the applicator pad 2810. The pad assembly 2880 can also include a backing portion 2818 that may be configured as a bandage. The piercing member 2899 can project distally from the applicator pad 2810 such that the release mechanism 2860 can be aligned with the piercing member 2899 (e.g., by handling the grip 2840) and pushed over the piercing member 2899 such that the piercing member 2899 pierces the release mechanism 2860 and allows liquid to flow from the reservoir 2850 through a lumen of the piercing member 2899 and/or outside of the piercing member through one or more openings defined in the plate 2841 to the applicator pad 2810. In some embodiments, the piercing member 2899 can be coupled to the plate 2841. In some embodiments, the piercing member 2899 can be monolithically formed with the plate 2841. The backing portion 2818 can be disposed in contact with a proximal surface of the plate 2841 and can extend away from the plate 2841 and the applicator pad 2810 in two or more opposing directions. The backing portion 2818 can include an adhesive covered pre-use by a peel-away adhesive liner disposed on a distal surface of the backing portion 2818. The adhesive liner can be peeled away prior to placing the backing portion 2818 on a subject's skin so that the backing portion 2818 can be adhered to the skin via the adhesive. The piercing member 2899, the plate 2841, and the backing portion 2818 may be the same or similar in structure and/or function to any of the piercing members, plates, and backing portions, respectively, described herein.

In some embodiments, the reservoir 2850 can include a connector 2881 disposed outside of the neck 2852 and configured to be releasably engaged with a fluid coupling 2855 (also referred to as a connector) of the pad assembly 2880 disposed outside of the piercing member 2899. The fluid coupling 2855 can be formed as a cylindrical structure surrounding the piercing member 2899 and projecting farther from the plate 2841 than the piercing member 2899 to prevent contact between a user (e.g., a user's finger) and the piercing member 2899. The fluid coupling 2855 can optionally be monolithically formed with the plate 2841 and/or the piercing member 2899. The connector 2881 and the fluid coupling 2855 can be configured as mating connectors, such as Luer connectors (as shown in FIGS. 32A-32L), threaded connectors, or connectors configured to mate via a friction fit. The connector 2881 and the fluid coupling 2855 can be configured such that the rotational direction the first portion 2842 is configured to turn relative to the fluid coupling 2855 to securely engage the connector 2881 with the fluid coupling 2855 is the same rotational direction the second portion 2844 is configured to turn relative to the first portion 2842 to advance the plunger 2858 such that the reservoir 2850 does not accidentally decouple from the pad assembly 2880 during a dispensing operation.

In use, the user can begin with the system 2800 in an initial configuration (e.g., a packaged configuration) in which the applicator device 2870 is decoupled from the pad assembly 2880. In the initial configuration, the reservoir 2850 can be filled with liquid (e.g., a medication and/or saline) and the plunger 2858 can be in an initial retracted configuration relative to the barrel 2854. To wet the applicator pad 2810, the user can hold the grip 2840 (e.g., one handed), align the release mechanism 2860 with the piercing member 2899 of the pad assembly 2880, and push the release mechanism 2860 over the piercing member 2899 such that the piercing member 2899 breaks the release mechanism 2860. Simultaneously or subsequently, the connector 2881 can optionally be engaged with the fluid coupling 2855 (e.g., by rotating a first Luer mating connector relative to a second Luer mating connector). The second portion 2844 can then be rotated relative to the first portion 2842 (e.g., by a thumb of the user) to advance the second portion 2844 distally, causing the plunger 2858 to be pushed distally relative to the first portion 2842 and the barrel 2854 to urge the liquid from the barrel 2854, through the neck 2852, through the fluid coupling 2855 and/or an optional lumen of the piercing member 2899, and to the applicator pad 2810. The pad assembly 2880 can be coupled to a patient such that the applicator pad 2810 is disposed in contact with a wound of the patient before or after coupling the pad assembly 2880 to the applicator device 2870 and before or after rotating the second portion 2844 to advance the plunger 2858 to wet the applicator pad 2810. After the fluid has been dispensed from the reservoir 2850, the grip 2840 and reservoir 2850 can optionally be decoupled from the pad assembly 2880 as shown in FIGS. 32I-32L by decoupling the fluid coupling 2855 from the connector 2881. The applicator device 2870 can be discarded and the pad assembly 2880 can remain in place in contact with the wound for a period of time. In some embodiments, the user can apply additional pressure to the pad assembly 2880 using the grip 2840 and/or to the pad assembly 2880 using a hand of the user after decoupling the grip 2840 from the pad assembly 2880 such that compression is applied to the wound by the applicator pad 2810 similarly as described with respect to other embodiments herein.

In some implementations, rather than the piercing member 2899 being coupled to the applicator pad 2810 and releasably couplable to the reservoir 2850, the piercing member 2899 can be coupled to the first portion 2842 similarly as is shown in other applicator devices described herein, such as the applicator device 2570 or the applicator device 2770. The applicator device 2870 could be engaged with the applicator pad 2810 via any suitable connector or via contact between a distal end of the first portion 2842 and the applicator pad 2810.

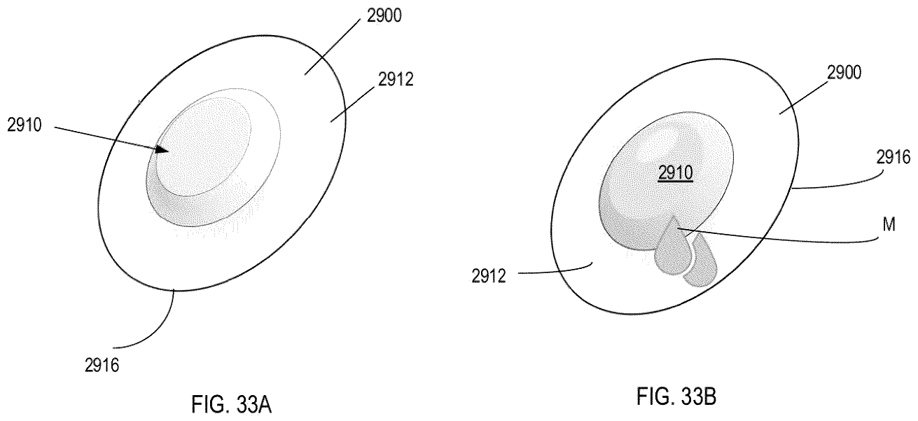

In some implementations a bleeding treatment system may include an applicator pad filled with a medication. FIGS. 33A and 33B show respective perspective views of a top and bottom of the bleeding treatment system 2900. The bleeding treatment system 2900 includes an applicator pad 2910 and a backing 2912. The applicator pad 2910 is filled with a medication M (e.g., any of the medications described herein) and the backing 2912 is configured to adhere to a tissue of a user. The backing 2912 may form a perimeter ring around the applicator pad 2910, as shown in FIGS. 33A and 33B. The backing 2912 may be similar to the bandage 1412, as shown in FIGS. 16A-16C. Also, in some cases, the distal side of the applicator pad 2910 may be configured to adhere to a skin of the user, while transmitting the medication M through the distal side of the applicator pad 2910 towards the tissue. The medication M may be any suitable medication for treating a wound or a surface of a skin of a user, such as any of the medications described herein (e.g., TXA, antibiotics, and the like or combination thereof).

In some embodiments, the medication M can be stored within the applicator pad 2910, which may be formed from a material containing, absorbing, wicking, or otherwise storing the medication M for application to a surface of the wound. The applicator pad 2910 may include a sponge, a flexible enclosure containing the medication M, and/or the like. In some implementations, the applicator pad 2910 can be formed from a flexible plastic, rubber, gauze, sponge soaked in the medication M, and/or the like.

In some embodiments, the applicator pad 2910 can have a level of saturation (e.g., can be pre-soaked to a saturation level) intended for a particular use of the bleeding treatment system 2900). In some embodiments, the volume and material of the applicator pad 2910 can be selected such that the applicator pad 2910 is about 35% saturated (i.e., contains about 35% of the amount of the medication(s) by volume or by weight). In some embodiments, the applicator pad 2910 can be configured to be between about 30% to and about 60% saturated by volume or by weight by the amount of the medication(s). In some embodiments, the applicator pad 2910 is configured to be between about 25% and about 50% saturated by volume or by weight by the amount of the medication(s). In some embodiments, the applicator pad 2910 may be pre-soaked with medication to about 40% of the saturation point of the applicator pad 2910. Alternatively, in some embodiments, the applicator pad may be pre-soaked with medication to about 50%, about 60%, about 70%, about 80%, or about 90% of the saturation point of the applicator pad 2910.

In some embodiments, the applicator pad 2910 can be pre-soaked or filled to hold a metered dose of medication. In some embodiments, the applicator pad 2910 can be configured such that the volume of medication(s) is effective to treat the wound but such that the medication does not travel across the user's skin outside of the treatment area (e.g., down a user's arm or leg) For instance, the applicator pad 2910 may be formed from material(s) with spatially (e.g., radially) variable saturation by medicine. In an example implementation, the applicator pad 2910 may be more saturated at the central portion of the applicator pad 2910 and less saturated near the perimeter of the applicator pad 2910.

In an example embodiment shown in FIGS. 33A and 33B, the backing 2912 forms a ring-shaped perimeter surrounding the applicator pad 2910. The backing 2912 may be non-permeable to fluid (e.g., to the medication M within the applicator pad 2910), thereby when the medication is released from the applicator pad 2910, it is not leaked through the backing 2912 (e.g., the medication is either contained within the applicator pad 2910 or being released into a wound or a skin surface of a patient). In some cases, the backing 2912 may only partially surround the applicator pad or may have shape other than the ring shape as shown in FIGS. 33A and 33B. For example, the backing 2912 may have extended portions and short portions, the extended portions configured to extends further away from the applicator pad 2910 than the short portions. As previously mentioned, the distal side of the applicator pad 2910 (i.e., the side of the applicator pad 2910 that is configured to be in contact with a wound surface or skin surface of a patient) may include adhesive such that the applicator pad 2910 can be adhesively coupled to a target wound area (e.g., a skin surface surrounding a wound) of the patient, or may have a portion (e.g., the portion may be located proximate to a perimeter of the applicator pad) that includes adhesive.

The bleeding treatment system 2900 is convenient as it can be readily used at various settings (e.g., at home, hospital, and the like), and during and after various procedures (e.g., the bleeding treatment system 2900 may be applied after surgery, including dermatological surgery such as Mohs micrographic surgery, punch biopsy, shave biopsy, and the like). The bleeding treatment system 2900 may be used post tracheostomy, post laceration repair with sutures as a means to protect the wound and stop bleeding, or after any other procedure that can cause tissue bleeding. Further, the bleeding treatment system 2900 may be used to treat various skin conditions (e.g., melasma, or any other suitable skin conditions). The bleeding treatment system 2900 may be used for patients with a higher risk of bleeding.

While the bleeding treatment system 2900 is shown to have a substantially circular surface of the applicator pad 2910, in some embodiments, any other shape of the applicator pad 2910 may be used (e.g., the bleeding treatment system 2900 may be configured to have different shapes to be used with respect to mucosal membranes including membranes within orifices such as a mouth, nose, ear, rectum, or vagina). The bleeding treatment system 2900 may be used as a therapeutic bandage having different shapes (e.g., circular, rectangular, elliptical, square, cross shape, and the like), and can be placed over user's forehead, cheeks, a nasal bridge, and the like.

In some embodiments, the top surface of the bleeding treatment system 2900 (e.g., the backing portion 2912) can be impermeable to the medication M contained within the applicator pad 2910. In some embodiments, the top surface of the bleeding treatment system 2900 can prevent drying or evaporation of the medication M from the applicator pad 2910.

In some implementations, the applicator pad 2910 may not be pre-soaked with the medication M, but instead the bleeding treatment system 2900 may include a bladder pre-filled with the medication M and adjacent to (or in proximity of) the applicator pad 2910 (e.g., the bladder may be disposed between the backing 2912 of the bleeding treatment system 2900 and the applicator pad 2910). The bladder can be configured to selectively release the medication M to the applicator pad 2910. In some implementations, the bladder can release the medication M to the applicator pad 2910 when it is ruptured due to a pressure applied to the bladder being greater than a threshold pressure (e.g., due to a user pressing on the bladder when the system 2900 is coupled to a wound). In some implementations, the bladder may include a valve that is configured to open when a pressure above a threshold pressure is applied to the bladder, thereby releasing the medication M to the applicator pad 2910.

In some embodiments, at least a bottom portion of the applicator pad 2910 (e.g., a surface of the applicator pad 2910 that is to be placed in contact with a wound of a user) can be covered or enclosed by the removable applicator seal to prevent contamination of the applicator pad 2910 and/or prevent undesired release of any medication(s) that may be contained in the applicator pad 2910. The applicator seal can be formed as a film or other thin layer of material non-reactive with the applicator pad 2910. The applicator seal can be removed from the applicator pad 2910 prior to use (e.g., via peeling) so that the medication M can be delivered to the wound tissue with which the surface of the applicator pad 2910 is placed in contact.

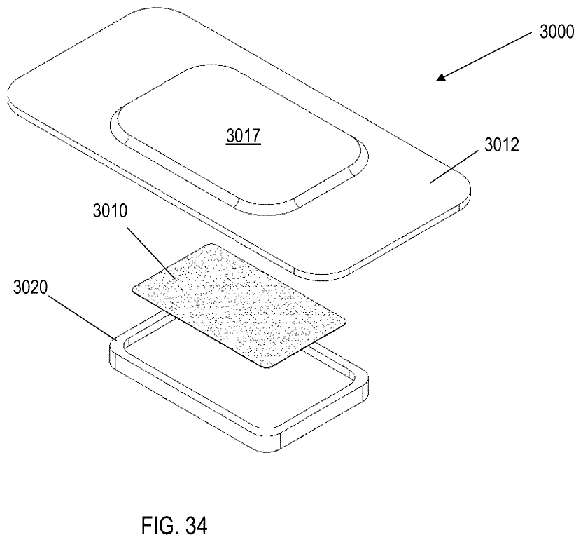

FIG. 34 shows a variation of a bleeding treatment system 3000, which may be similar to the bleeding treatment system 2900. The bleeding treatment system 3000 includes an applicator pad 3010, an applicator seal 3020, and a backing 3012 which may be similar to the respective applicator pad 2910 and the backing 2912 (with a difference that the applicator pad 3010 and the backing 3012 have a rectangular shape). In the example embodiment, the applicator pad 3010 may be formed from a material containing, absorbing, wicking, or otherwise transporting one or more medications for application to a surface of the wound (e.g., the applicator pad 3010 may be a sponge). The applicator pad 3010 may be soaked (e.g., pre-soaked) in a medication (e.g., TXA, antibiotics, or any other suitable medication for treating a tissue). Further, to prevent drying and/or evaporation of the medication from the applicator pad 3010, the applicator seal 3020 can be used to seal the applicator pad 3010 (e.g., to hermetically seal applicator pad 3010) in a space defined between the applicator seal 3020 and the backing 3012. In some implementations, the applicator seal 3020 can be formed as a rigid or flexible cover that can be releasably coupled to the backing 3012 and can enclose the applicator pad 3010.

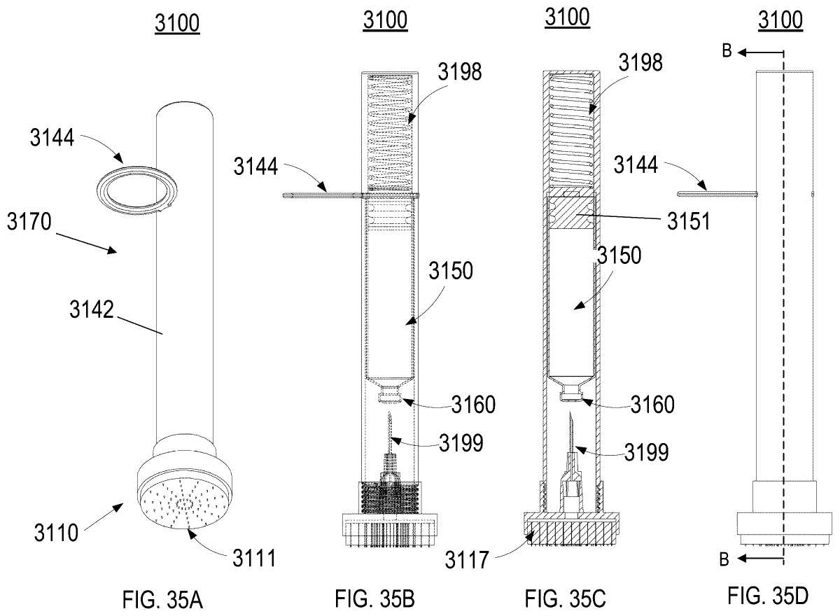

FIGS. 35A-35D show an example embodiment of a bleeding treatment system 3100 which may be similar to any of the bleeding treatment systems described herein, such as the bleeding treatment system 2500. FIG. 35A is a perspective view, FIG. 35B is a side view showing internal components of the bleeding treatment system 3100, FIG. 35C is a cross-sectional view taken along line B-B as shown in FIG. 35D, and FIG. 35D is a side view of the bleeding treatment system 3100. In the example embodiment, various portions, components, etc. of the bleeding treatment system 3100 may be the same or similar to respective portions, components, etc. of any of the bleeding treatment systems described herein, such as the bleeding treatment system 2500. For example, the bleeding treatment system 3100 includes an applicator device 3170 having a first portion 3142. A reservoir 3150 is located within the first portion 3142. The reservoir 3150 and the first portion 3142 may be respectively the same as the reservoir 2550 and the first portion 2542 of the applicator device 2570. The applicator device 3170 includes a piercing member 3199 and a release mechanism 3160, as shown in FIGS. 35B-35C. Further, the bleeding treatment system 3100 includes an activation mechanism 3198, a retaining pin 3144 for releasably retaining the activation mechanism 3198, and a piston 3151 configured to be moved by the activation mechanism 3198 and to cause the reservoir 3150 to move towards the piercing member 3199 to pierce the release mechanism 3160 when the retaining pin 3144 is removed. These components may be the same as respective components of the bleeding treatment system 2500, for example, the bleeding treatment system 3100 may include an applicator pad 3110 including an array of microneedles 3111.

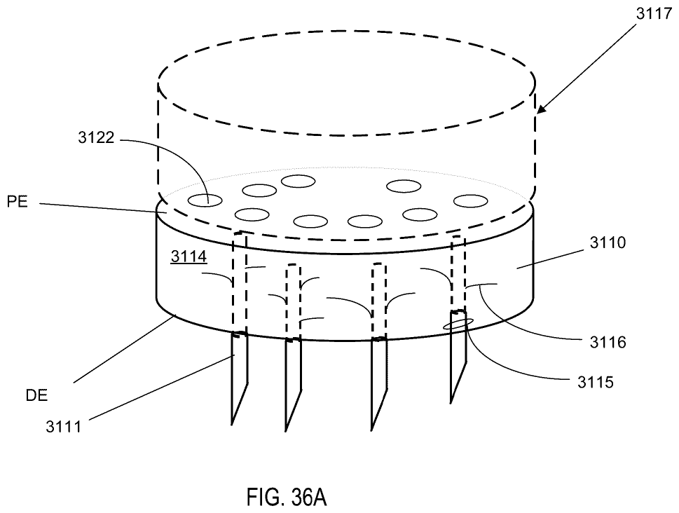

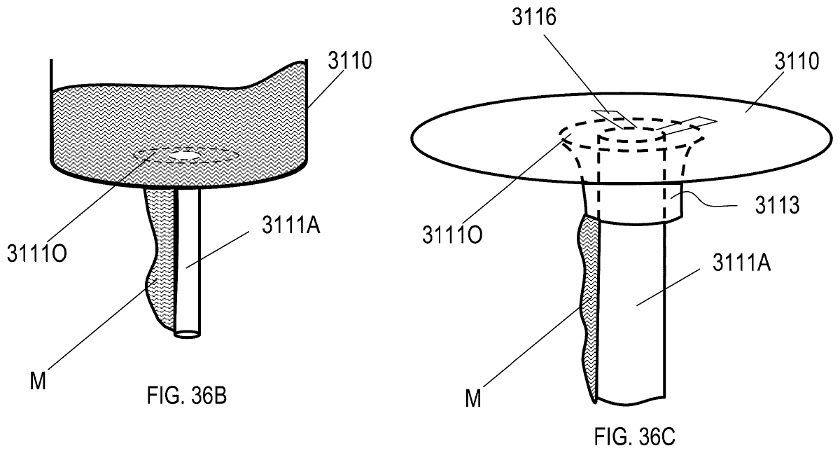

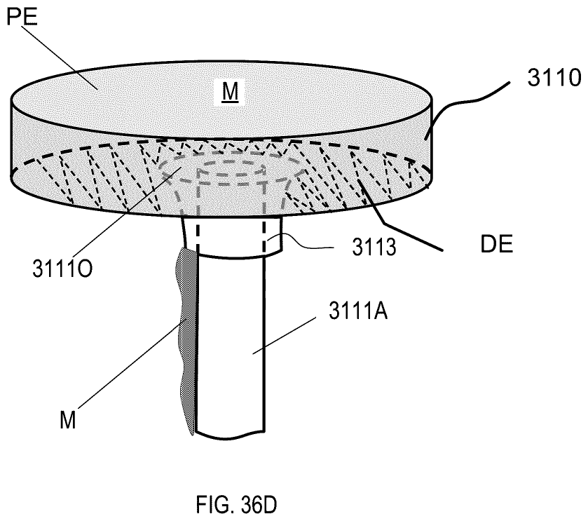

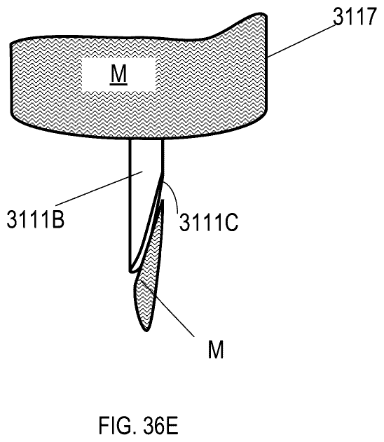

An example of a portion of the bleeding treatment system 3100 is shown in FIG. 36A. The bleeding treatment system 3100 includes an applicator pad 3110, which may be formed from a material containing, absorbing, wicking, or otherwise transporting one or more medications for application to a surface of the wound (e.g., the applicator pad 3110 may be a sponge). The applicator pad 3110 may be similar to the applicator pad 2510 of the bleeding treatment system 2500. Further, the microneedles 3111 may be attached, affixed or coupled to the applicator pad 3110 using any suitable coupling elements. In one implementation, the microneedles 3111 may be affixed directly to a distal end (DE) of the applicator pad 3110 via coupling elements 3115. Alternatively, the microneedles 3111 may be attached to a material forming the applicator pad 3110 via coupling elements 3116, as shown in FIG. 36A. In some optional implementations, the applicator pad 3110 may be adjacent to an enclosure 3117 containing a medication (e.g., the medication M) such that the medication M flows from the enclosure 3117 towards the applicator pad 3110 via openings 3122 in the proximal side of the enclosure 3117.

In some implementations, the DE of the applicator pad 3110 may be configured to be impenetrable to the medication M in at least some regions (e.g., regions between the microneedles 3111, but may be configured to be penetrable to the medication M in proximity of the microneedles 3111). Alternatively, the entirety of the DE may be configured to be penetrable to the medication M. Further, in some implementations, none of the DE may be configured to be penetrable to the medication M, and the medication M may be flowed to a tissue of a user (e.g., a skin of the user) via channels within the microneedles 3111, as further described below.

In various embodiments, using the bleeding treatment system 3100 includes pressing the applicator pad 3110 against the tissue requiring treatment (e.g., the bleeding treatment system 3100 can be pressed against the outer surface of tissue (e.g., the skin) so that the microneedles pierce the skin). The bleeding treatment system 3100 can then be actuated (before or after urging the microneedles through the tissue surface) such that the medication M flows through the applicator pad 3110 and into the pierced holes in the patient to a layer of tissue (e.g., dermis layer) that is deeper than the tissue surface (e.g., skin surface). The actuation includes removing the retaining pin 3144, thereby releasing the activation mechanism 3198.

After releasing the medication M into the tissue, the applicator pad 3110 may be decoupled, disconnected, detached, removed, and the like, from the bleeding treatment system 3100 at a proximal end PE, while being coupled (e.g., attached, adhered, and the like) to a surface of the tissue of the user. In one implementation, similar to various applicator pads described above, the applicator pad 3110 may be adhered to a top surface of the tissue (e.g., to a top surface of user's skin) using any suitable adhering element (e.g., an adhesive bandage). While being attached to the tissue, the microneedles 3111 of the applicator pad 3110 remain attached to a patient for a period of time (as a patch) after the initial attachment/actuation.

The applicator pad 3110 may include any suitable number of microneedles 3111 facilitating transporting the medication M to tissues of a user. For example, the applicator pad 3110 may include a few tens of microneedles or as much as a hundred or a few hundreds of microneedles. In one example, the number of microneedles may be selected such that a characteristic distance between neighboring microneedles is a fraction of a millimeter, about a millimeter, a few millimeters, and the like. In some cases, the distance between the neighboring microneedles may be about the same as a diameter of a microneedle, about a few diameters of a microneedle, or about a few tens of diameters of the microneedle.

The microneedles 3111 may be arranged in any suitable way. For example, the microneedles may be arranged in a rectangular grid, a triangular grid, a hexagonal arrangement, in a form of concentric circles, checkerboard arrangement, or any other suitable periodic or aperiodic arrangements. In the example embodiment, as shown in FIG. 35A, the microneedles 3111 are arranged in concentric circles with each circle having about the same number of microneedles (with the exception of a single microneedle in the middle of the applicator pad 3110). Alternatively, the microneedles 3111 may be arranged in a concentric circles with a number of microneedles in each circle proportional to a radius of the circle (e.g., a separation distance between the microneedles 3111 in any particular circle may be about constant). It should be appreciated that any other suitable arrangement of the microneedles 3111 may be selected. The microneedles 3111 may be made from any suitable material (e.g., fluoropolymer, plastic, metal, glass, ceramics, and the like) and may have any suitable length for extending into the dermis and/or epidermis. In some implementations, the microneedles may be a fraction of a millimeter long or may be as long as a few millimeters (e.g., 4 millimeters (mm)). In some implementations, the microneedles may be 0.2 mm, 0.4 mm, 0.6 mm, 0.8 mm, 1 mm, 1.2 mm, 1.4 mm, 1.6 mm, 1.8 mm, 2 mm, 2.2 mm, 2.4 mm, 2.6 mm, 2.8 mm, 3 mm, 3.2 mm, 3.4 mm, 3.6 mm, 3.8 mm, 4 mm, and the like. The microneedles may have any suitable shape (e.g., a conical shape, a cylindrical shape having a sharp tip, or any other suitable shape).

FIGS. 36B and 36C show a first implementation of a microneedle 3111A for the bleeding treatment system 3100. In a first implementation, the microneedle 3111A may be a solid protrusion with a sharp tip. As shown in FIG. 36B, an opening 31110 is formed at a bottom side of the applicator pad 3110 about the microneedle 3111A. The opening 31110 is configured to allow a medication M to flow from the applicator pad 3110 along an outside surface of the microneedle 3111A. The applicator pad 3110 may be filled with the medication M once the retaining pin 3144 is released and the activation mechanism 3198 causes the reservoir 3150 to move towards the piercing member 3199, such that the medication M is released from the reservoir 3150 into the enclosure 3110.

FIG. 36C, shows an example embodiment, in which the opening 31110 includes a nozzle element 3113 configured to guide the medication M along the outside surface of the microneedle 3111A. The nozzle element 3113 is configured to surround the microneedle 3111A (e.g., the microneedle 3111A is placed in the middle of the nozzle element 3113). In the example embodiment, the microneedle 3111A is attached to a bottom surface of the applicator pad 3110 via coupling elements 3116.

FIG. 36D shows an implementation of the bleeding treatment system 3100 similar to the implementation shown in FIGS. 36B and 36C including the applicator pad 3110. The applicator pad 3110 includes a distal end DE that may not (or may be) penetrable by medication M. Further, the applicator pad 3110 includes an opening 31110 through which the medication M may flow from the applicator pad 3110 towards an outer wall of the microneedle 3111A (e.g., the medication M may flow from the applicator pad 3110 via passage formed by the nozzle 3113, as shown in FIG. 36D). The microneedle 3111A may be attached to the applicator pad 3118 using any suitable coupling elements (e.g., coupling elements that are similar to the coupling elements 3116, as shown in FIG. 36C). For example, such coupling elements may be configured to attach the microneedle 311A to a proximal end PE of the applicator pad 3110, to a distal end DE of the applicator pad 3110, or to a material within the applicator pad 3110. In some cases, a portion of the outer surface of the microneedle 3111A may be glued, or otherwise adhered to the material of the applicator pad 3110.

In some cases, the applicator pad 3110 may be deformable/compressible so that, as microneedles (e.g., the microneedles 3111) are pushed through the patient's tissue and the applicator pad 3110 contacts a surface of a user's tissue (e.g., a surface of a skin of the user), the applicator pad 3110 retracts relative to the microneedles, thereby exposing more of the length of the microneedles 3111 (so that the microneedles 3111 can be urged deeper in the tissue). For such a configuration, the microneedles 3111 may be attached to the applicator pad 3110 at a top portion of the applicator pad 3110, such that the bottom portion of the applicator pad 3110 can move relative to the microneedles 3111.

When the bleeding treatment system 3100 is actuated to soak the applicator pad 3110 prior to urging the microneedles through the tissue surface and when the applicator pad 3110 is compressible, the retraction of the applicator pad 3110 relative to the microneedles 3111 may also cause medication M in the soaked applicator pad 3110 to be released (e.g., squeezed) from the applicator pad 3110 to contact the tissue surface and flow into the channels formed by the microneedles 3111 piercing the tissue of the user.

Alternatively, in some embodiments, the applicator pad 3110 may be substantially rigid and/or incompressible so that the distance the microneedles 3111 extend beyond the applicator pad 3110 does not change when the bleeding treatment system 3100 is pressed against the user's tissue. For such an embodiment, the distance the microneedles 3111 extend beyond the applicator pad 3110 prior to use of the bleeding treatment system 3100 is equal to the insertion depth of the microneedles 3111.

FIG. 36E shows another implementation of a microneedle 3111B. The microneedle 3111B may be a hollow structure (e.g., a protrusion containing a lumen forming a channel between the applicator pad 3110 and an outflow opening 3111C of the microneedle 3111B). The formed channel is configured to flow the medication M from the applicator pad 3110 into the epidermis, and, in some cases, dermis of the patient. In some cases, the applicator pad 3110 may be configured not to allow a quick transfer of medication M from the applicator pad 3110 through a bottom surface of the applicator pad 3110 (e.g., the bottom surface of the applicator pad 3110 may be configured not to transfer the medication M towards a wound of a user and may be in contact with the wound) so that the medication Mis flown through the microneedles coupled to the bottom of the applicator pad 3110, rather than through the bottom surface of the applicator pad 3110 upon activation of the bleeding treatment system 3100.

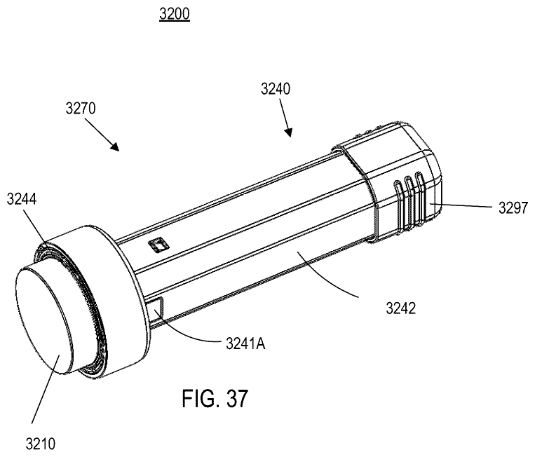

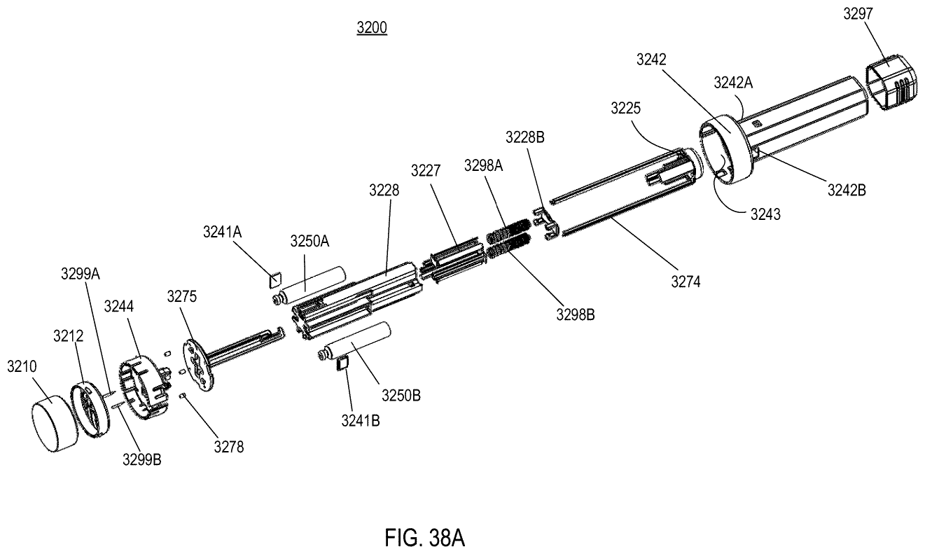

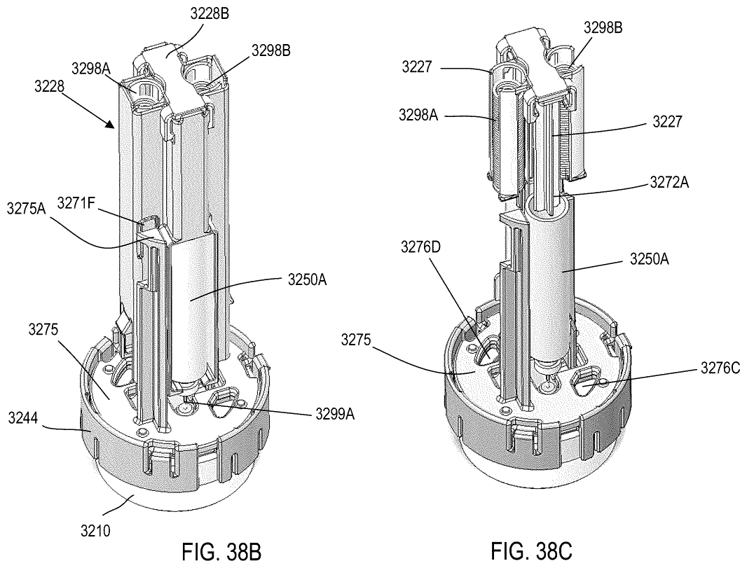

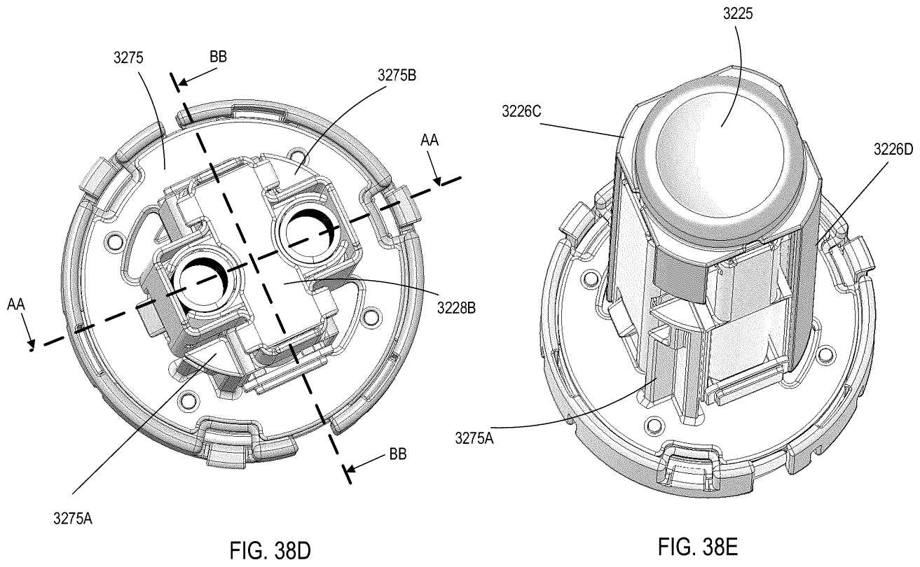

FIGS. 37-50 are various views of an example embodiment of a bleeding treatment system 3200. FIG. 37 is a perspective view, and FIG. 38A is an exploded view, respectively, of the bleeding treatment system 3200. The bleeding treatment system 3200 can be the same or similar in structure and/or function to any of the bleeding treatment systems described herein. For example, the bleeding treatment system 3200 includes an applicator device 3270 including a grip 3240 (also referred to as, or including, a housing or an outer housing) and an applicator pad 3210, which can be the same or similar in structure and/or function to any of the grips and applicator pads described herein. For example, the applicator pad 3210 may be structurally or functionally similar to the applicator pad 2310 as shown schematically in FIG. 27. The grip 3240 includes a first portion 3242 and a second portion 3244. As shown, the first portion 3242 (also referred to as a first housing portion) can be formed as an elongated housing portion having an open distal end and define an interior space 3243. The second portion 3244 (also referred to as a second housing portion or a lower housing portion) can be disposed at least partially within the interior space 3243. The applicator pad 3210 can be coupled to the second portion 3244 (e.g., coupled to a distal-facing surface of the second portion 3244 and/or can be disposed within an interior space defined by the second portion 3244) such that at least a portion of the applicator pad 3210 is disposed distal of the second portion 3244 and distal of the first portion 3244. The applicator pad 3210 can include or be coupled to a backing 3212 such that the applicator pad 3210 is coupled to the second portion 3244 via the backing 3212.

In some embodiments, the bleeding treatment system 3200 is configured to be used by a single hand of a user (e.g., the subject having a wound or a caregiver of the subject). For example, the bleeding treatment system 3200 may be picked up by a left or a right hand of the user at the first portion 3242 (e.g., wrapping a user's fingers around the first portion 3242 and about a central axis of the first portion), and the applicator pad 3210 may be placed in contact with a wound. The user can translate the first portion 3242 toward the wound by pushing or pulling the first portion 3242 with the user's hand. Sufficient translation of the first portion 3242 relative to the second portion 3244 resulting from translating the first portion 3242 toward the wound causes the applicator pad 3210 to be compressed against the wound, causes compression to be applied to the wound, and causes the system 3200 to transition from an initial, unactuated configuration to an actuated configuration such that fluid (e.g., medication) is delivered to the applicator pad 3210. As the user continues to maintain the applicator pad 3210 against the wound (e.g., compressing the applicator pad 3210), the fluid travels to the wound and/or blood is absorbed from the wound into the applicator pad 3210. In some implementations, the applicator pad 3210 includes a seal that can be removed from the applicator pad 3210 prior to placing the applicator pad 3210 in contact with the wound of the subject.

FIG. 38A shows an exploded view of the bleeding treatment system 3200 illustrating various components of the bleeding treatment system 3200. Besides the applicator pad 3210, the first portion 3242 and the second portion 3244, as were previously shown in FIG. 37, the interior space 3243 defined by the first housing portion 3242 includes components for facilitating the release of the fluid into the applicator pad 3210. Further, the various coupling components and elements are also shown in FIG. 38A. The bleeding treatment system 3200 includes a proximal cap 3297, an applicator release mechanism 3274 (also referred to as an applicator pad release mechanism) located at a top portion within the interior space 3243, a cap 3225 (also referred to as an ejector button), an activation mechanism 3298 formed by springs 3298A and 3298B, a drive plunger 3227, an inner housing 3228 (also referred to as an inner support frame), reservoirs 3250A and 3250B, housing windows 3241A and 3241B, an actuator slider 3275, a fluid coupler carrier 3244 (also referred to as a second or lower housing portion) coupled to the actuator slider 3275 via couplers 3278, piercing members 3299A and 3299B coupled to the fluid coupler carrier 3244, and an applicator cap 3212 (also referred to as a backing or backing portion) configured to at least partially housing the applicator pad 3210.

The bleeding treatment system 3200 includes an applicator release mechanism 3274 located at a top portion within the interior space 3243. The applicator release mechanism 3274 may be configured to move distally (i.e., toward the applicator pad 3210) and relative to the first portion 3242 and the second portion 3244 to push the applicator cap 3212 and the applicator pad 3210 coupled to the applicator cap 3212 from the remainder of the system 3200 (e.g., from the first portion 3242 and the second portion 3242). In some implementations, the applicator release mechanism 3274 may be moved distally due to a distal movement of the cap 3225 (e.g., due to a user pressing on the cap 3225). As can be seen in FIG. 38E, the applicator release mechanism 3274 can include elongated members 3226C and 3226D extending distally from the proximal end of the applicator release mechanism 3274 towards the applicator cap 3212. These elongated members 3226 are configured to push the applicator cap 3212 out of engagement with the second portion 3244 when the applicator release mechanism 3274 moves relative to the first portion 3242 and the second portion 3244.

The inner housing 3228 is located within the interior space 3243 of the first portion 3242 and is configured to house the springs 3298A and 3298B, the drive plunger 3227, and the reservoirs 3250A and 3250B. FIGS. 38B-38E show further details of the inner housing 3228 and various elements proximal to the inner housing 3228 with the first portion 3242 not shown. For example, FIG. 38B shows a perspective view of the inner housing 3228, FIG. 38C shows the elements located within the inner housing 3228 without the inner housing 3228 shown, FIG. 38D shows a top view of the system 3200 with the first portion 3242, cap 3297, and applicator release mechanism 3274 not shown. FIG. 38E shows a top view of the system 3200 with the first portion 3242 not shown but the applicator release mechanism 3274 included.

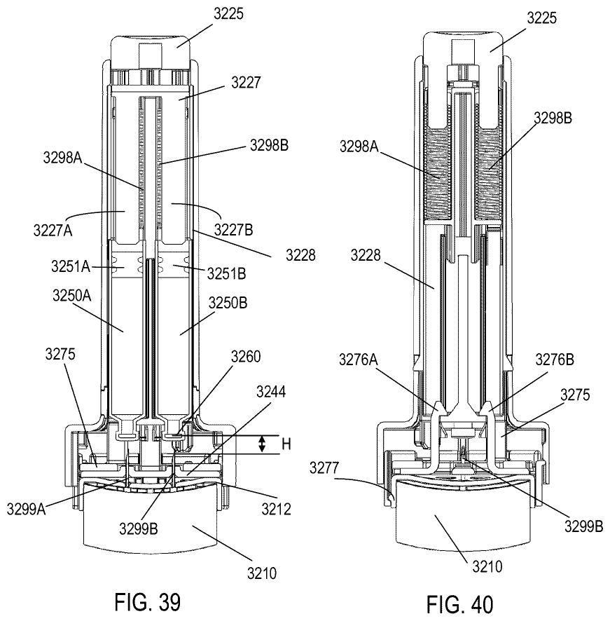

Prior to actuation, the drive plunger 3227 is disposed at a top portion of the inner housing 3228 as shown, for example, in FIG. 38B. The drive plunger 3227 includes a plunger 3227A disposed above and aligned with a piston 3251A of the reservoir 3250A and a plunger 3227B (shown in FIG. 39) disposed above and aligned with a piston 3251B of the reservoir 3250B. Each of the plungers 3227A and 3227B is configured to be translated relative to reservoir housings (e.g., barrels) of the respective reservoirs 3250A and 3250B and to engage with the respective pistons 3251A and 3251B to urge the pistons 3251A and 3251B distally to cause the contents of the reservoirs 3250A and 3250B to be urged distally through and out of the distal end of the reservoir housings and to the applicator pad 3210 (e.g., via respective piercing members 3299A and 3299B).

The drive plunger 3227 houses at least a portion of springs 3298A and 3298B, as shown, for example, in FIG. 38C. In some implementations, the springs 3298A and 3298B can be retained in a compressed state in the initial, unactuated configuration of the bleeding treatment system 3200 and can be configured to expand when released from the compressed state to urge the drive plunger 3227 to move downwards (e.g., distally towards the applicator pad 3210). In some implementations, rather than using springs 3298A and 3298B as an activation mechanism for releasing and/or driving fluid from the reservoirs 3250A and 3250B, other activation mechanisms can be used that store a mechanical or electrical energy to selectively translate the drive plunger 3227 distally. Such activation mechanisms may include, for example, flexible elements (e.g., flexible membranes), strings under extensions, wound up springs, electrical batteries coupled to electrical motors, pneumatic devices (e.g., pneumatic chambers containing compressed gas) or any other suitable electrical or mechanical devices that can cause the motion of the drive plunger 3227.

An upper cap 3228B can be fixedly coupled to the proximal end of the inner housing 3228. The inner housing 3228 retains the reservoirs 3250A and 3250B within an interior of the inner housing 3228 such that the reservoirs 3250A and 3250B are in a fixed position relative to the inner housing 3228 (and, thus, are in a fixed position relative to the first portion 3242). In some implementations, rather than including two reservoirs, the system 3200 can include only one reservoir. Each reservoir 3250A and 3250B has an open distal end closed by a release mechanism 3260 (e.g., a pierceable membrane such as a cap or cover). In the initial configuration of the system 3200, the release mechanisms 3260 can be disposed proximal of the piercing members 3299. In the actuated configuration of the system 3200, the piercing members 3299 can pierce the release mechanisms 3260 to release fluid from the reservoirs 3250.

The reservoirs 3250A and 3250B may be made from a transparent material (e.g., glass, transparent plastic, and the like), and the inner housing 3228 may include transparent windows 3241A and 3241A through which the reservoirs 3250A and 3250B can be viewed to observe the operation of the system 3200 (e.g., to determine the amount of fluid remaining within the reservoirs 3250A and 3250B). In some cases, the windows 3241A and 3241B may include markings indicating the volume of medicine remaining with the reservoirs 3250A and 3250B based on the visible location of the pistons 3251A and 3251B. Alternatively, the reservoirs 3250A and 3250B may include markings, and windows 3241A and 3241B may be openings defined by the inner housing 3228. The windows 3241A and 3241B may be disposed within openings 3242A and 3242B defined within the first portion 3242, as shown in FIG. 38A.

The inner housing 3228 can include flexible detents (e.g., a flexible detent 3271F is shown in FIG. 38B) that can be moved from an initial position to a second position in response to a movement of the actuator slider 3275 and associated protrusions 3275A and 3275B. In some implementations, a single flexible detent may be used. Alternatively, a plurality of flexible detents may be used. For example, in implementations including protrusions 3275A and 3275B, two flexible detents may be used. In some implementations, the system 3200 can include a flexible detent associated with each protrusion.

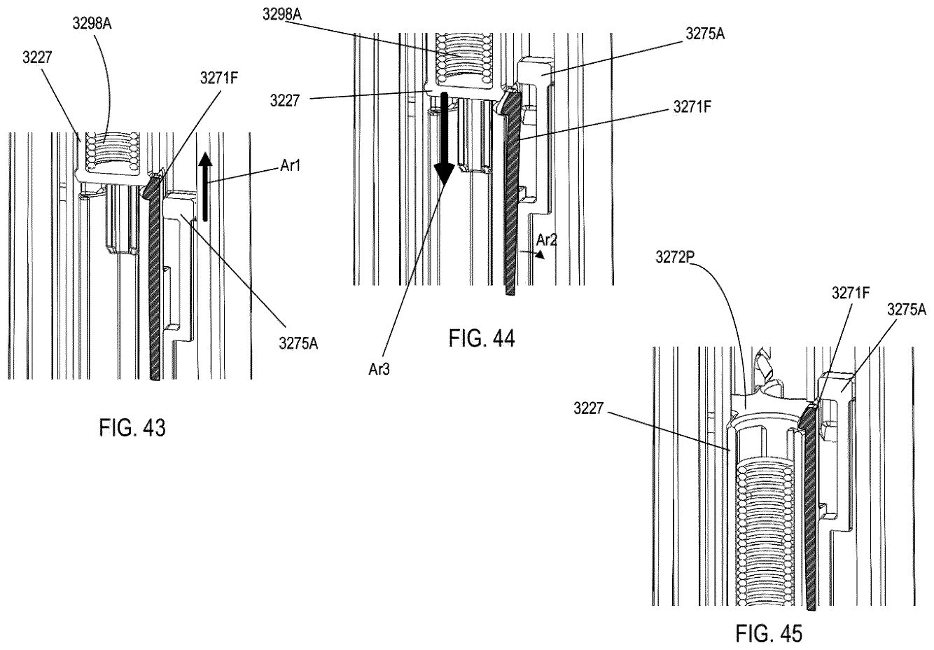

The protrusions 3275A and 3275B are configured to be translated along the walls of the inner housing 3228 in response to the first portion 3242 being translated distally relative to the second portion 3244, causing an activation action of springs 3298A and 3298B. For example, the sliding of the protrusions 3275A and 3275B proximally relative to the first portion 3242 causes movement of the flexible detents (as further described below in connection to FIGS. 43-45), which causes a release of the drive plunger 3227 and springs 3298A and 3298B.

The actuator slider 3275 is coupled to the fluid coupler carrier 3244 via coupling elements 3278, which can be screws, rivets, or any other suitable coupling elements for fixedly attaching the actuator slider 3275 to the fluid coupler carrier 3244. In some implementations, rather than being separate components, the actuator slider 3275 and the fluid coupler carrier 3244 may be monolithically formed from any suitable material (e.g., plastic, metal, composite material, ceramics, glass, and the like).

The fluid coupler carrier 3244 is disposed within a distal end of the first portion 3242 and is configured to slide proximally relative to the first portion 3242 and the inner housing 3228 when a user presses the applicator pad 3210 onto a wound. Further, the actuator slide 3275 being fixedly attached to the fluid coupler carrier 3244 is also configured to slide proximally by the same distance as the fluid coupler carrier 3244.

The second portion 3244 includes fixedly attached piercing members 3299A and 3299B. In some implementations, the piercing members 3299A and 3299B can form fluid couplings and may be formed as hollow needles such that the piercing members 3299A and 3299B define channels between the respective reservoirs 3250A and 3250B and the applicator pad 3210 when the second portion 3244 is translated proximally relative to the first portion 3242. In some implementations, the distal ends of the piercing members 3299A and 3299B can be disposed within openings defined in the backing portion 3212 and/or can be coupled to the applicator pad 3210, and the proximal ends of the piercing members 3299A and 3299B can be configured to puncture release mechanisms of respective reservoirs 3250A and 3250B when the fluid coupler carrier 3244 slides proximally relative to the first portion 3242 (e.g., the piercing members 3299A and 3299B may be each positioned below the release mechanisms 3260 of respective reservoirs 3250A and 3250B). In some embodiments, the number of fluid couplings can correspond to the number of fluid reservoirs. For example, if in an implementation a single reservoir is used, then a single fluid coupling (e.g., piercing member) can be employed for forming a channel between the reservoir and the applicator pad 3210. If more than two reservoirs are used, then more than two fluid couplings (e.g., piercing members) can be employed.

The second portion 3244 can include a first set of detents configured to couple (e.g., via a snap fit) to the inner housing 3228 after sliding proximally relative to the first portion 3242, thereby preventing the second portion 3244 from sliding back distally relative to the first portion 3242. Additionally, or alternatively, the second portion 3244 may include another (e.g., second) set of detents for coupling (e.g., via snap fit) to a distal portion of the first portion 3242, thereby preventing the second portion 3244 from sliding back distally relative to the first portion 3242. Further, after moving the second portion 3244 proximally relative to the first portion 3242 and engaging the first set of detents to couple to the inner housing 3228 and/or the second set of detents to couple to the distal portion of the first portion 3242, a tactile and audible feedback may be delivered to the user indicating that the bleeding treatment system 3200 is activated.

The second portion 3244 can be coupled to the applicator cap 3212. The applicator cap 3212 can be snap fit to the second portion 3244. The applicator cap 3212 includes a proximal side adjacent to the second portion 3244 and a distal side adjacent to the applicator pad 3210. Further, the applicator cap 3212 defines openings through which the piercing members 3299A and 3299B can be disposed and/or with which the piercing members 3299A and 3299B can be aligned such that fluid can flow from the reservoirs 3250 to the applicator pad 3210 via the openings in the applicator cap 3212. The applicator cap 3212 may be decoupled from the second portion 3244 by applying a sufficient force onto the proximal side of the applicator cap 3212, thereby pushing the applicator cap 3212 from the second portion 3244. In some implementations, the second portion 3244 can be coupled to the slider 3275, which includes a base plate including openings 3276C and 3276D through which respective protrusions 3274C and 3274D are configured to translate into contact with the proximal side of the applicator cap 3212 when the applicator release mechanism 3274 moves distally relative to the first portion 3242. The exerted force by protrusions 3274C and 3274D can cause the applicator cap 3212 to decouple from the second portion 3244.

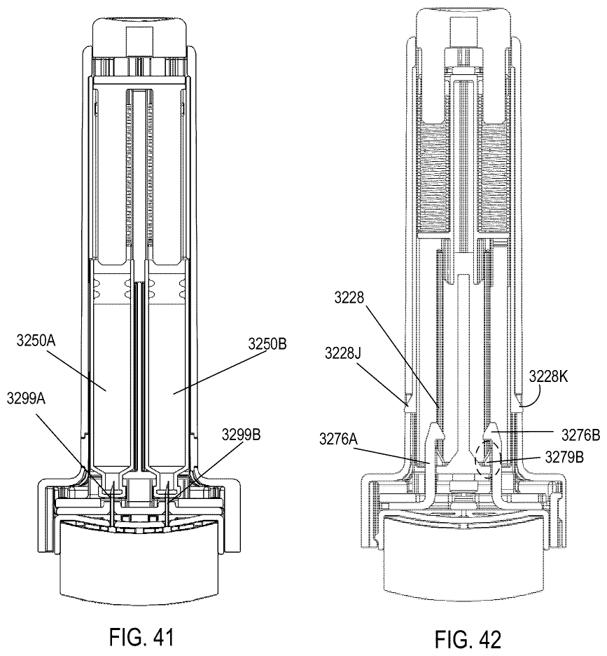

FIGS. 39 and 40 show cross-sectional views of the bleeding treatment system 3200 in an initial, unactuated configuration and FIGS. 41 and 42 show the same cross-sectional views of the bleeding treatment system 3200 after actuation (e.g., after the applicator pad 3210, the applicator cap 3212, and the second portion 3244 are moved proximally relative to the first portion 3242, thereby resulting in piercing members 3299A and 3299B penetrating release mechanisms 3260 of respective reservoirs 3250A and 3250B and resulting in springs 3298A and 3298B being released to drive the drive plunger 3227 to urge the pistons 3251A and 3251B of the reservoirs 3250A and 3250B distally). For example, FIG. 39 shows a cross-sectional view along a cross-sectional plane AA, as shown in FIG. 38D but including all components of the system 3200, and FIG. 40 shows a cross-sectional view along a cross-sectional plane BB, as shown in FIG. 38D but including all components of the system 3200.

FIGS. 39 and 40 shows the bleeding treatment system 3200 prior to the first portion 3242 being translated distally relative to the second portion 3244. For example, as shown in FIG. 39, the proximal ends of the piercing members 3299A and 3299B are separated from the distal ends of the respective reservoirs 3250A and 3250B (e.g., the release mechanisms 3260) by a distance H. As shown in FIGS. 39 and 40, the drive plunger 3227 is located in an initial plunger position such that distal ends of plungers 3227A and 3227B are disposed within or near proximal portions of respective reservoirs 3250A and 3250B.

As shown in FIG. 41, after sliding the first portion 3242 and the inner housing 3228 distally relative to the second portion 3244, the piercing members 3299A and 3299B are configured to penetrate the release mechanisms 3260 of respective reservoirs 3250A and 3250B, thereby forming a channel between these reservoirs and the applicator pad 3210 for fluid to flow from the reservoirs 3250A and 3250B to the applicator pad 3210.

FIG. 42 further shows details of detents 3276A and 3276B configured to couple (e.g., snap fit) to the housing 3228. In some cases, more than two detents may be used. For example, FIG. 42 shows a use of a third detent 3279B for coupling to the housing 3228. FIG. 42 also shows that detents 3228J and 3228K may be used for coupling the inner housing 3228 to the first portion 3228. It should be noted that FIGS. 41 and 42 show the drive plunger 3227 in the first plunger position prior to its movement into a second plunger position, as further discussed below.

FIGS. 43-45 show the flexible detent 3271F configured to bend into a first or a second position based on a movement of the actuator slider 3275 and the associated protrusion 3275A. In an example embodiment shown in FIG. 43, the flexible detent 3271F is located in a first position such that it retains the drive plunger 3227 and prevents the drive plunger 3227 from moving downwards (e.g., in a distal direction, towards the applicator pad 3210) due to action of the compressed spring 3298A. As shown in FIG. 43, the flexible detent 3271F is prevented from moving to a second position (e.g., bending laterally as indicated by an arrow Ar2) by the protrusion 3275A. However, when the protrusion 3275A slides upwards (e.g., in a proximal direction away from the applicator pad 3210), as indicated by an arrow Ar1 in FIG. 43, the flexible detent 3271F is configured to move into a second position. For example, the flexible detent 3271F is configured to bend out as shown in FIG. 44 by the arrow Ar2. The movement of the flexible detent 3271F into the second position eliminates the support for the drive plunger 3227, thus, resulting in the drive plunger 3227 moving downwards (e.g., towards the applicator pad 3210) due to the action of the compressed spring 3298A. In an example embodiment, the drive plunger 3227 is configured to move from the first plunger position, as shown in FIG. 43 and FIGS. 39-40 to a second plunger position as shown in FIG. 45. FIG. 45 shows that in the second plunger position, the drive plunger 3227 is moved downwards such that a proximal end 3272P of the drive plunger 3227 is located distally from the end of the flexible detent 3271F. When the drive plunger 3227 is in the second plunger position, the flexible detent 3271F is configured to move back into the first position, thereby securing the drive plunger 3227 in the second plunger position. It should be appreciated that a plurality of flexible detents may be used, and such flexible detents may be structurally and/or functionally similar to or the same as the flexible detent 3271F.

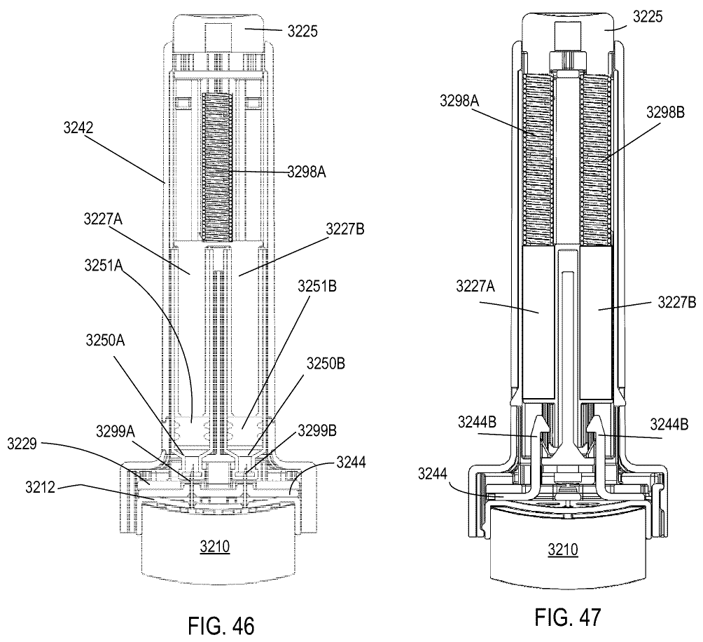

FIGS. 46 and 47 show cross-sectional views of the bleeding treatment system 3200 after the drive plunger 3272 moves into the second plunger position. FIG. 46 shows a cross-sectional view along a cross-sectional plane AA, as shown in FIG. 38D but including all components of system 3200, and FIG. 47 shows a cross-sectional view along a cross-sectional plane BB, as shown in FIG. 38D but including all components of system 3200. Note that the drive plunger 3227 is in the second plunger position such that distal ends of respective plungers 3227A and 3227B are disposed in contact with pistons 3251A and 3251B at the distal ends of respective reservoirs 3250A and 3250B, thereby having driven the fluid from the reservoirs 3250A and 3250B to the applicator pad 3210 (e.g., via the piercing members 3299A and 3299B). In some embodiments, when the drive plunger 3227 is in the second plunger position, most or all of the fluid initially disposed in the reservoirs 3250A and 3250B is displaced by plungers 3272A and 3272B and moved into the applicator pad 3210.

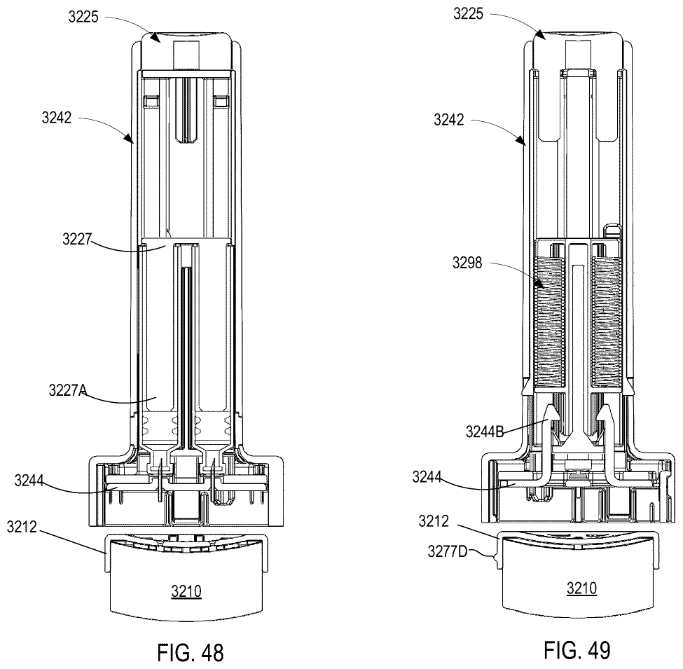

FIGS. 48 and 49 show cross-sectional views of the bleeding treatment system 3200 after the applicator cap 3212 and the applicator pad 3210 is removed from a distal end of a the first portion 3242 and the second portion 3244. FIG. 48 shows a cross-sectional view along a cross-sectional plane AA, as shown in FIG. 38D but with all components of the system 3200 included, and FIG. 49 shows a cross-sectional view along a cross-sectional plane BB, as shown in FIG. 38D but with all components of the system 3200 included. As shown in FIGS. 48 and 49, the applicator cap 3212 is configured to be disconnected from the second portion 3244 and separated from the first portion 3242. As shown in FIG. 49, the applicator cap 3212 may include a detent 3272D (or a plurality of detents similar to the detent 3272D) that can be used to releasably couple (e.g., via a snap fit) the applicator cap 3212 to the second portion 3244. The applicator cap 3212 may be formed from a flexible material, such that when a pressure is applied to the proximal side of the applicator cap 3212 (e.g., due to a movement of the applicator release mechanism 3274) the detent of the applicator cap 3212 is configured to decouple from the second portion 3244.

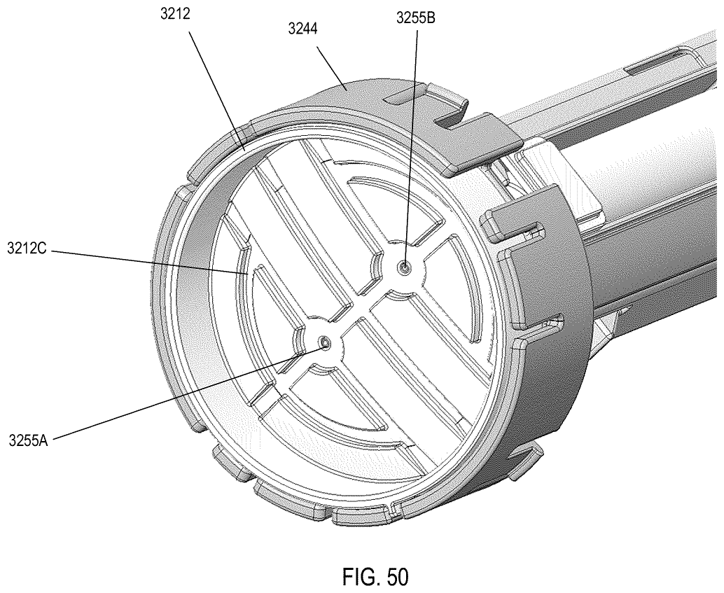

FIG. 50 shows a perspective view of a distal side of the applicator cap 3212 when engaged with the second portion 3244. The applicator cap 3212 defines openings to function as fluid couplings 3255A and 3255B through which fluid from the reservoirs 3250A and 3250B can flow to the applicator pad 3210. The distal surface of the applicator cap 3212 also defines fluid channels 3212C configured to allow the fluid from the reservoirs 3250A and 3250B to flow to disperse laterally relative to a proximal surface of the applicator pad 3210. In some embodiments, the fluid channels 3212C may be designed to spread over a top surface area of the applicator pad 3210 such that when a larger volume of fluid is dispensed to the applicator pad 3210 than can be immediately absorbed or dispensed by the applicator pad 3210, the fluid can flow into the channels 3212C and travel into the applicator pad 3210 at various locations. For example, as shown in FIG. 50, the cap 3212 can define three channels extending laterally in a first direction, one channel extending laterally in a second direction, perpendicular to the first direction, and two curved channels. It should be appreciated that other suitable channels may be used (e.g., any other number of channels of different shapes and directions may be used).

In some embodiments, the applicator pad 3210 can be configured to fit within the interior space defined by the applicator cap 3212. In some embodiments, the applicator pad 3210 may be formed as, or include, a sponge, which can be compressed when it is inserted within the applicator cap 3212 and/or when the applicator pad 3210 is pressed against a patient surface using the system 3200 is pressed against a patient surface.

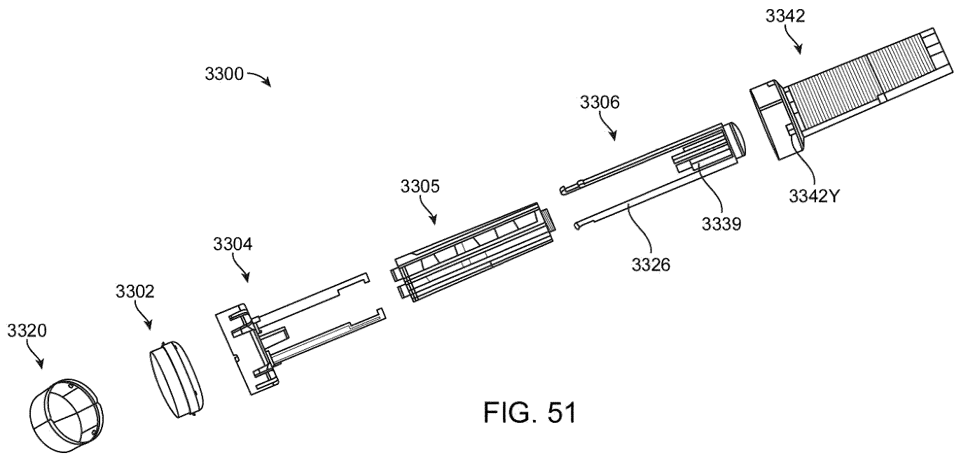

FIG. 51 is an exploded view of a bleeding treatment system 3300. The bleeding treatment system 3300 can be the same or similar in structure and/or function to any of the bleeding treatment systems described herein. The bleeding treatment system 3300 can include an outer housing 3342 (also referred to as an outer housing portion, a first housing, or a first housing portion), at least one reservoir 3350, an ejection subassembly 3306, an inner housing subassembly 3305, a lower actuator subassembly 3304, a sponge cup subassembly 3302, and an optional distal cap 3320.

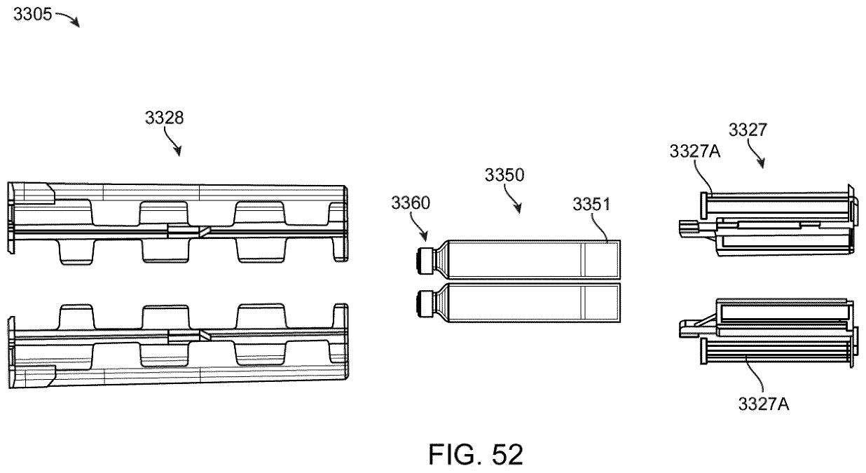

As shown in FIG. 52, the inner housing subassembly 3305 can include an inner support frame 3328 defining an enclosure including one or more channels configured to receive and retain the reservoirs 3350 (e.g., a pair of channels, each including a reservoir 3350 of a pair of reservoirs 3350) and to receive at least a portion of a drive plunger 3327. In some embodiments, the inner support frame 3328 can be formed of two or more separate components that can be fixedly coupled to each other and/or the outer housing 3342. In some embodiments, the inner support frame 3328 can be a unitary structure (e.g., monolithically formed). In some embodiments, the drive plunger 3327 can be formed of two separate components that can be coupled to each other and/or the inner support frame 3328. In some embodiments, the drive plunger 3327 can be a unitary structure (e.g., monolithically formed). The reservoirs 3350 can be included in the inner housing subassembly 3305 and can be retained in a fixed position relative to the inner support frame 3328. The reservoirs 3350 can each also be retained in a fixed position relative to the outer housing 3342 due to the inner support frame 3328 being coupled and retained in a fixed position along a central axis of the outer housing 3342 relative to the outer housing 3342.

The drive plunger 3327 can include a pair of plunger members 3327A (also referred to as plunger elements), each plunger member 3327A configured to be translated within the channels defined by the inner support frame 3328 to engage with respective reservoir 3350 of the pair of reservoirs 3350. For example, the reservoirs can be formed as cartridges including a reservoir housing (e.g., a barrel), a piston 3351 closing a proximal end of the reservoir housing, and a release mechanism 3360 (e.g., a pierceable membrane in the form of a cap or cover) closing a distal end of the reservoir housing. The drive plunger 3327 can be translated distally (e.g., within the inner support frame 3328) to urge each plunger member 3327A into engagement with a piston 3351 of each reservoir 3350 to advance the piston 3351 relative to a housing of each reservoir 3350 and urge fluid from the reservoir 3350.

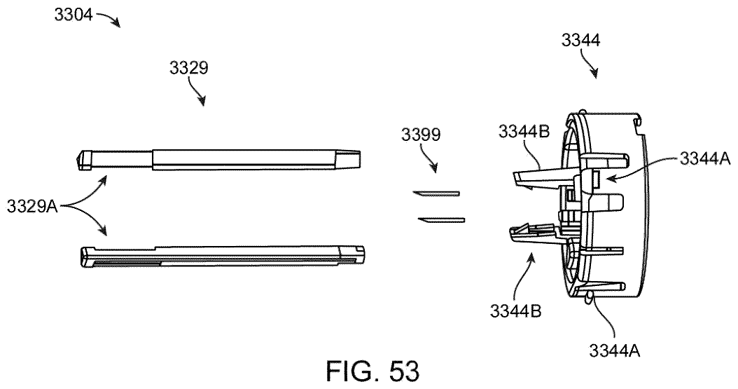

As shown in FIG. 53, the lower actuator subassembly 3304 can include a second housing portion 3344 (also referred to as a lower housing portion), a pair of piercing members 3399 (e.g., needles), and a pair of elongated members 3329. The second housing portion 3344 can include external engagement features 3344A (e.g., latches) and proximally-extending retention elements 3344B (e.g., latches). The proximally-extending retention elements 3344B are configured to be engaged with the inner support frame 3328 in an initial, unactuated configuration of the system 3300 to retain the second housing portion 3344 relative to the first housing portion 3342 via the inner support frame 3328. The external engagement features 3344A are configured to be matingly received within openings or recesses 3342Y defined in the outer housing 3342 to retain the second housing portion 3344 relative to the first housing portion 3342 after the second housing portion 3344 is translated proximally relative to the first housing portion 3342 during actuation of the system 3300.

The second housing portion 3344 also defines a pair of openings configured to receive the piercing members 3399 such that the needles are fixed within the openings and extend proximally from the openings. Each of the piercing members 3399 can have a sharp proximal end and a blunt distal end that does not contact any components other than the second housing portion 3344 during operation of the system 3300. The second housing portion 3344 is also configured to be coupled to the pair of elongated members 3329 such that the pair of elongated members 3329 extend proximally from the second housing portion 3344 and are fixed relative to the second housing portion 3344 such that translation of the lower actuator subassembly 3304 translates the pair of elongated members 3329. Each of the elongated members defines a recessed portion 3329A.

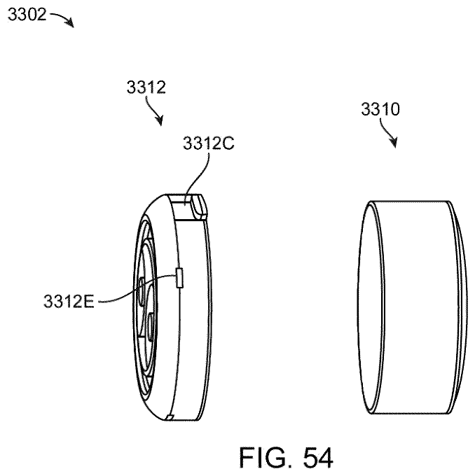

As shown in FIG. 54, the sponge cup subassembly 3302 (also referred to as a pad subassembly) can include an absorbent pad 3310 and a backing 3312 (also referred to as a backing portion, a cup, or a sponge cup). The backing 3312 can include one or more retention elements 3312C (e.g., latches) and one or more outwardly-extending projections 3312E. The retention elements 3312C can be configured to mate with an inwardly-facing retention flange or rim disposed on an inner surface of a sidewall of the second housing portion 3344 to prevent the backing 3312 from separating from the second housing portion 3344 inadvertently prior to an optional ejection step. The outwardly-extending projections 3312E can be configured to mate with a recess or groove defined in an inwardly-facing surface of the second housing portion 3344 such that when a proximal force is applied to the backing portion 3312 (e.g., by the applicator pad 3310 due to pressing the applicator pad 3310 against a surface of a subject while urging the first housing portion 3342 distally toward the surface), the backing portion 3312 urges the second housing portion 3344 proximally into the interior of the first housing portion 3342.

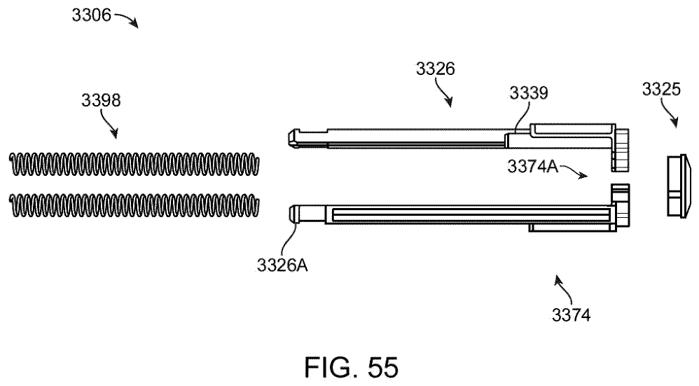

As shown in FIG. 55, the ejection subassembly 3306 includes an ejection button 3325 coupled to or included in an applicator pad release mechanism 3374. The applicator pad release mechanism 3374 includes a pair of pad-urging elongated members 3326 coupled to the ejection button 3325 (e.g., via a base portion 3374A) and extending distally a sufficient distance such that the elongated members 3326 can contact and urge the backing 3312 distally when the ejection button 3325 is depressed, a pair of spring-receiving elongated members 3339, and an actuation mechanism 3398 (also referred to as energy storage element(s)) formed by a pair of spring elements 3398 configured to be threaded over the spring-receiving elongated members 3339. In some embodiments, the actuation mechanism 3398 can be included separately from the ejection subassembly 3306. The elongated members 3326 include retention elements 3326A disposed near a distal end of each elongated member 3326 and configured to selective engagement with the second housing portion 3344. Additionally, the elongated members 3326 include distal ends configured to engage with a portion of the backing 3312 such that, when the elongated members 3326 are advanced relative to the second housing portion 3344 due to a force applied to the button 3325 (e.g., by translating through one or more openings of the second housing portion 3344), the distal ends of the elongated members 3326 can apply a force to the backing 3312 to dislodge the backing 3312 from the second housing portion 3344 and separate the backing 3312 and the applicator pad 3310 from the remainder of the system 3300. In some embodiments, the elongated members 3326, elongated members 3339, base portion 3374A, and/or button 3325 can be formed of two, three or more separate components that can be fixedly coupled to each other. For example, as shown in FIG. 55, the elongated members 3325, elongated members 3339, and base portion 3374A can be formed as a unitary structure. In some embodiments, the elongated members 3326, elongated members 3339, base portion 3374A, and/or button 3325 can be formed as a unitary structure (e.g., monolithically formed).

The spring elements 3398 can be disposed such that a proximal end of each spring is in contact with a distal-facing surface of the base portion 3374A and a distal end of each spring element 3398 is in contact with a proximal-facing surface of the drive plunger 3327 such that actuation of the system 3300 (e.g., via translating the second housing portion 3344 proximally relative to the first housing portion 3342 to cause the drive plunger 3327 to be released from an initial position) causes the spring elements 3398 to transition from a stored energy configuration to an activated configuration (e.g., from a compressed to an expanding and/or expanded configuration) to drive the drive plunger 3327 distally (e.g., to urge the pistons 3351 of the reservoirs 3350 distally). In some embodiments, the actuation of the system 3300 can also cause (e.g., simultaneously or immediately before or after) the button 3325 to be transitioned to an actuated position from an initial position (e.g., translated proximally relative to the first housing portion 3342) due to the proximal end of the spring elements 3398 applying a proximal force to the base portion 3374A and/or the button 3325 such that the button 3325 can be pressed distally by the user to urge the elongated members 3398 into urging contact with the backing 3312 to urge the applicator pad 3310 and the backing 3312 away from the second housing portion 3344.

In some embodiments, the cap 3320 can include two cap halves coupled together via a flexible or living hinge. Thus, the cap 3320 can be separated from the distal end of the remainder of the system 3320 via bending or rotating one half relative to the other half of the cap 3320.

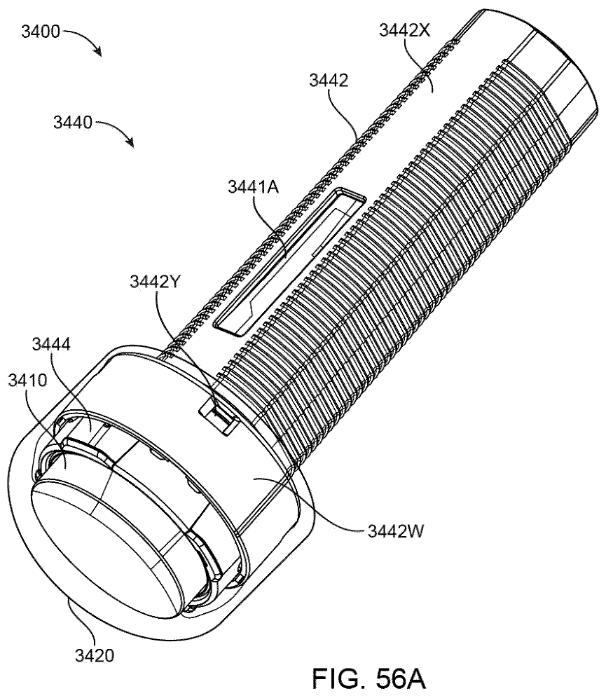

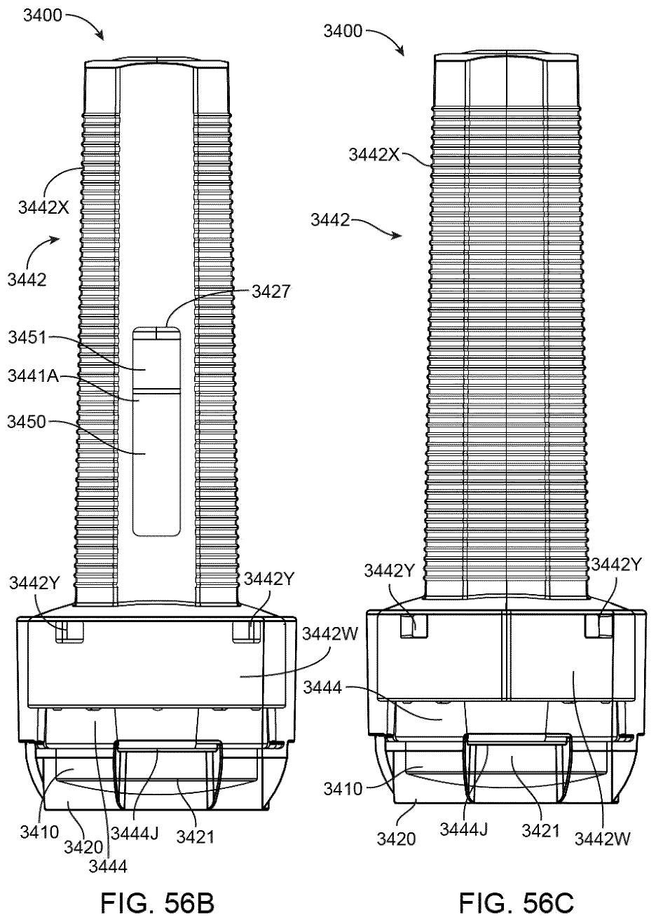

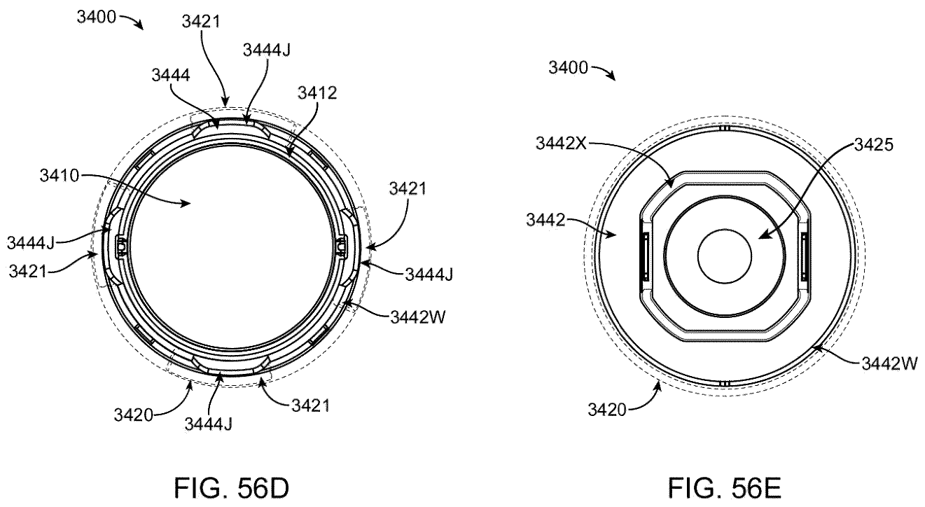

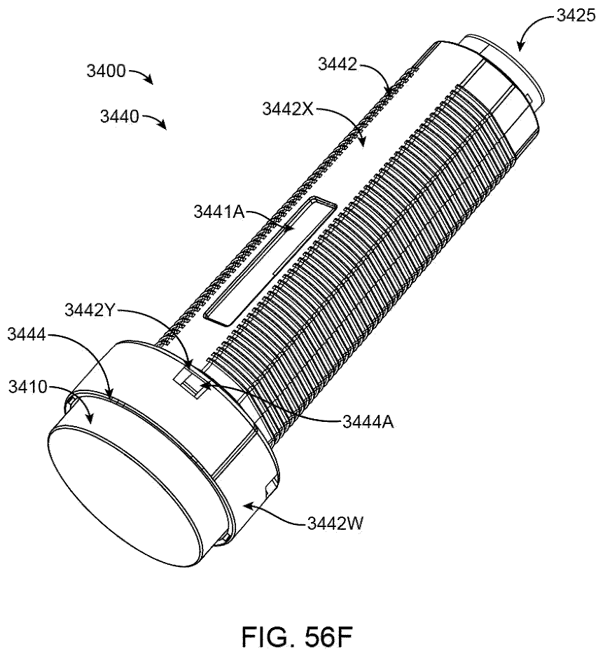

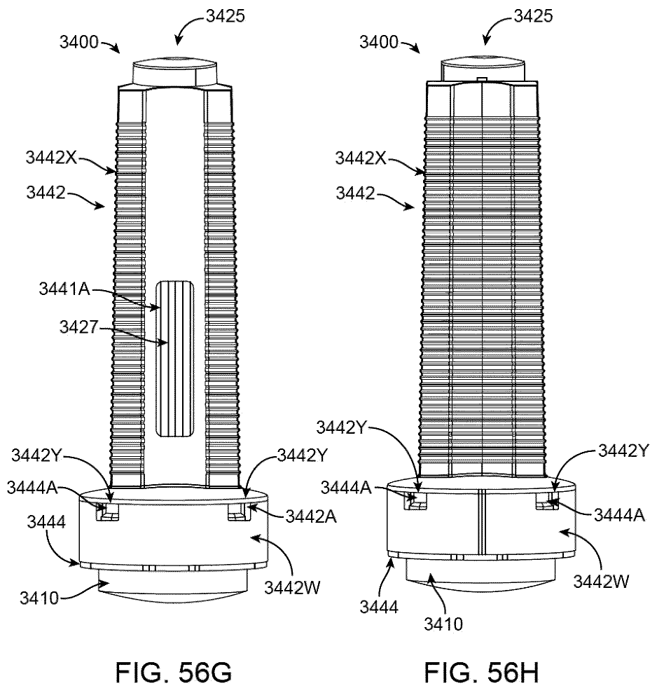

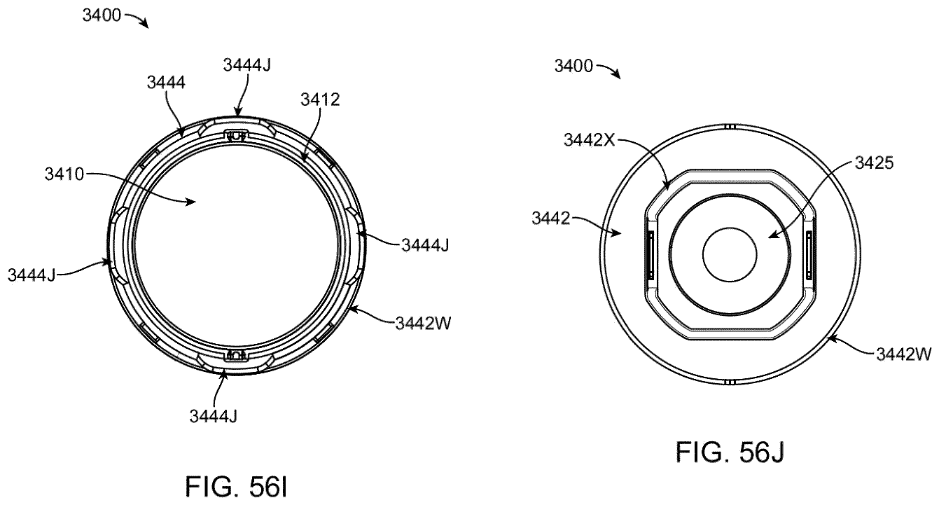

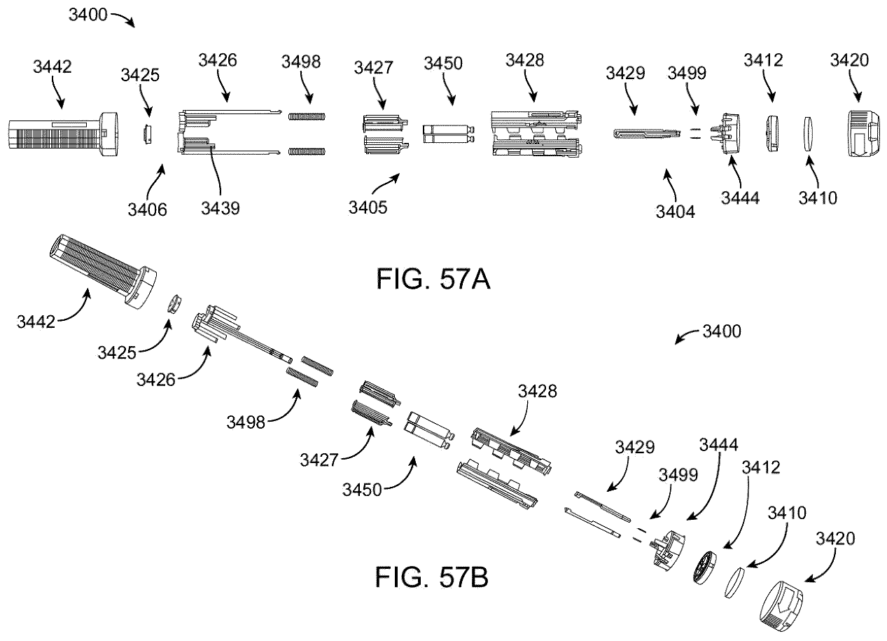

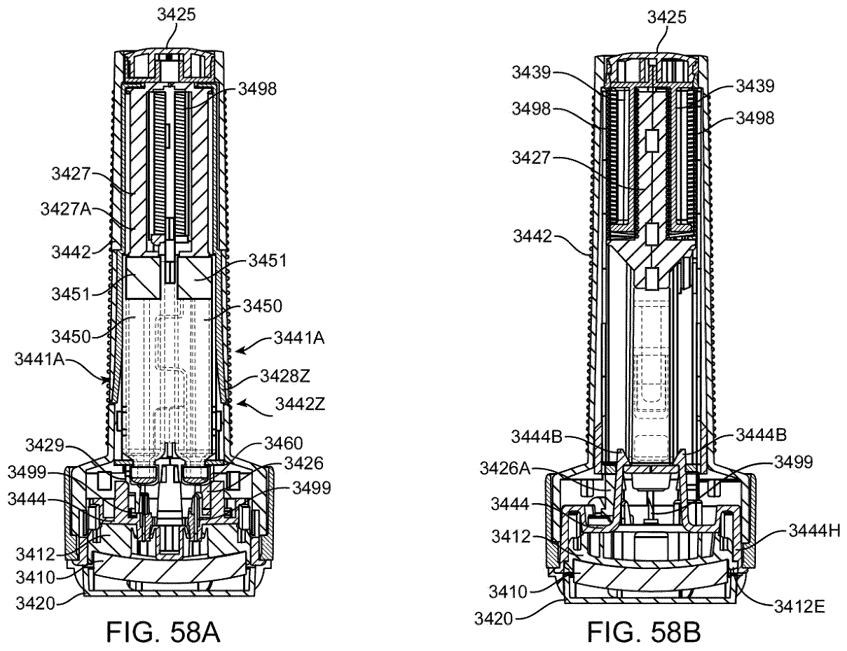

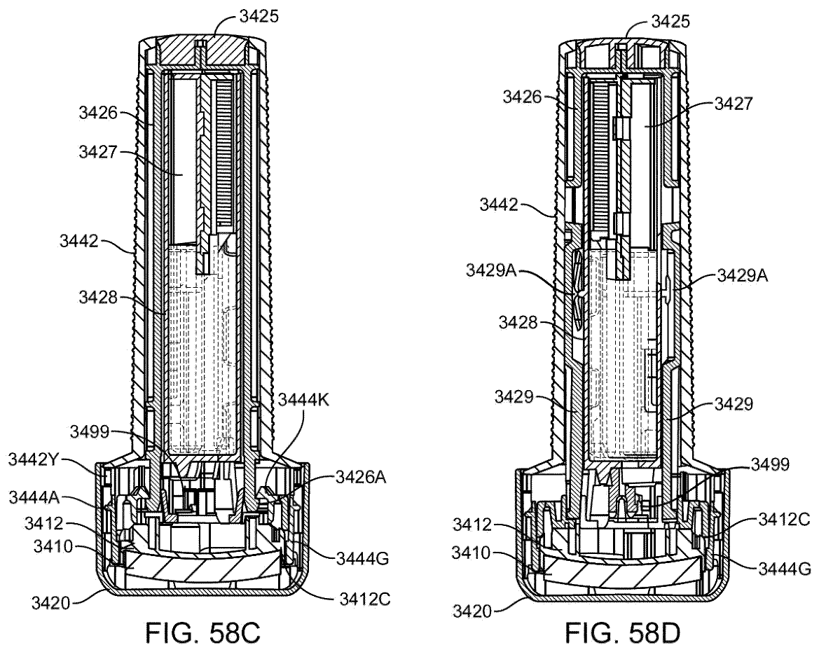

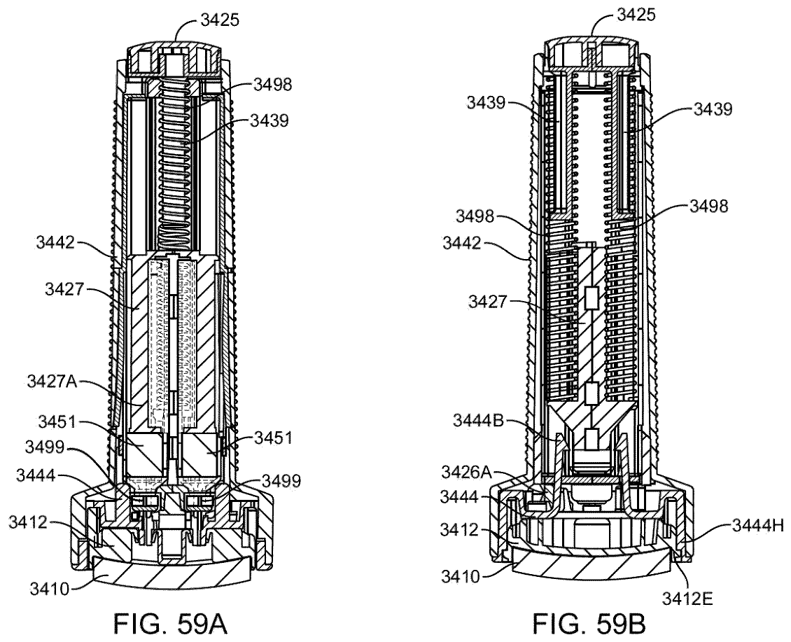

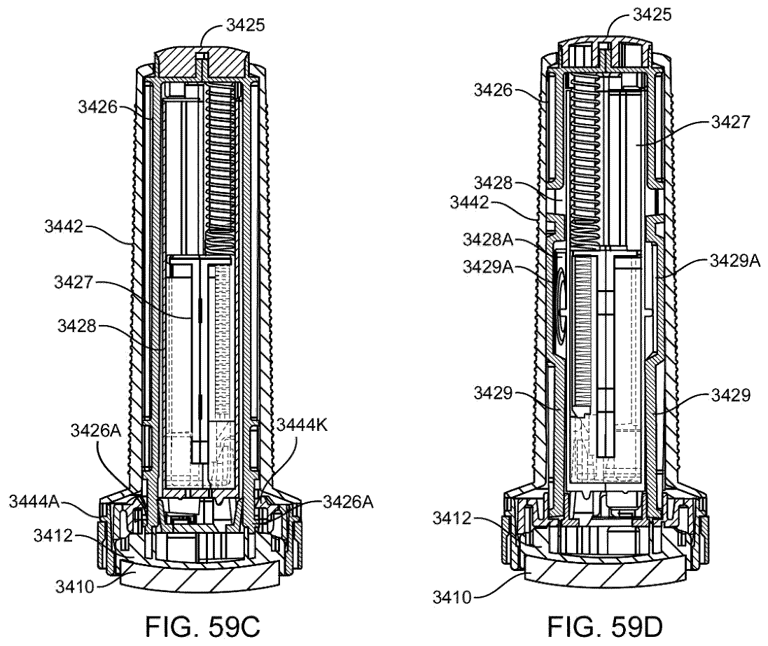

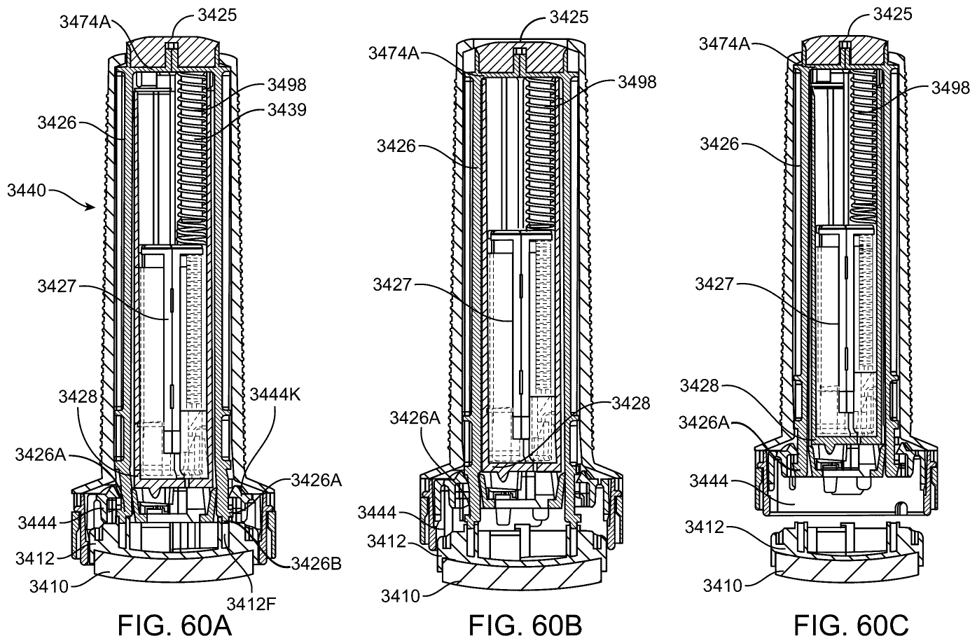

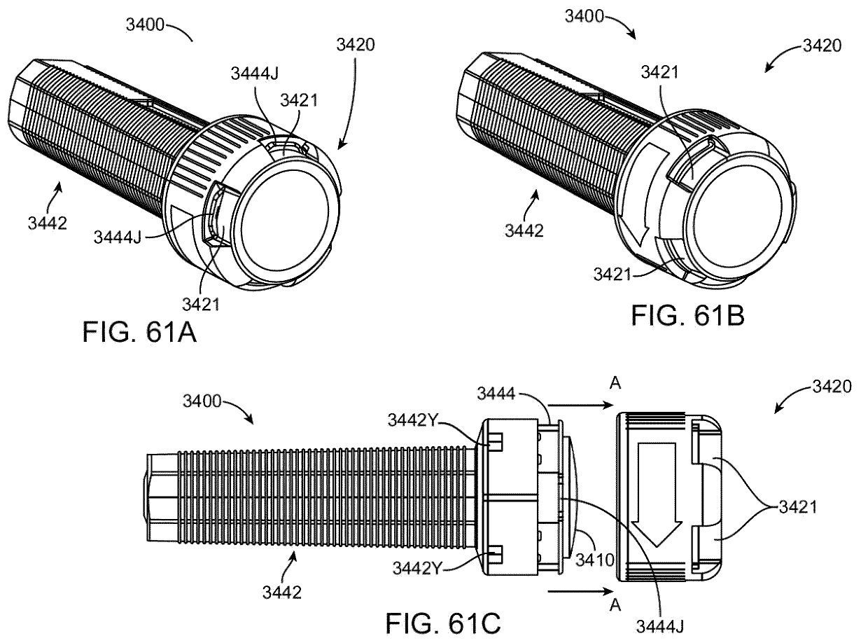

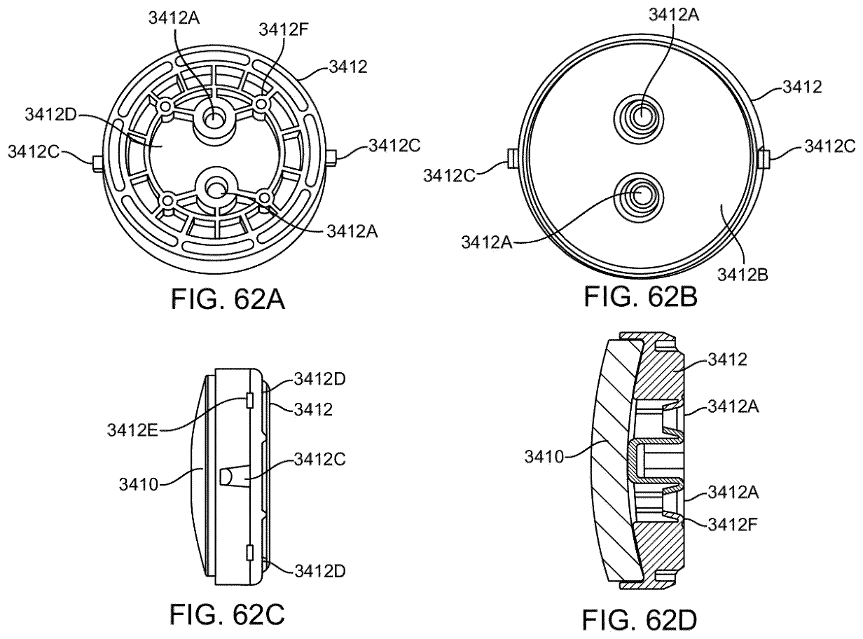

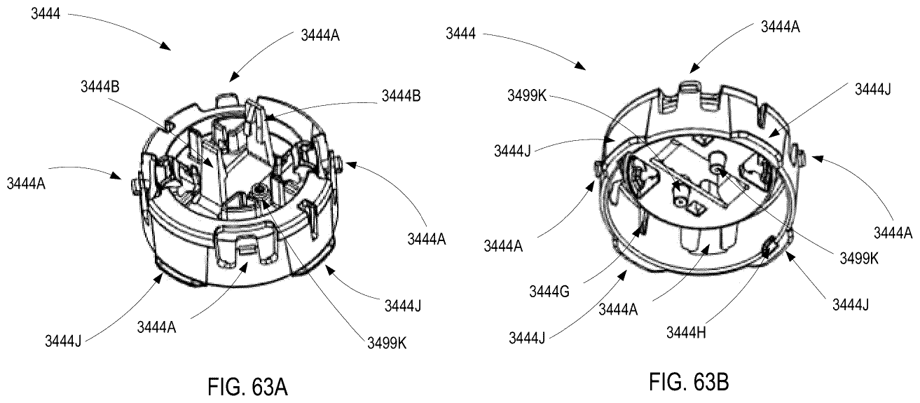

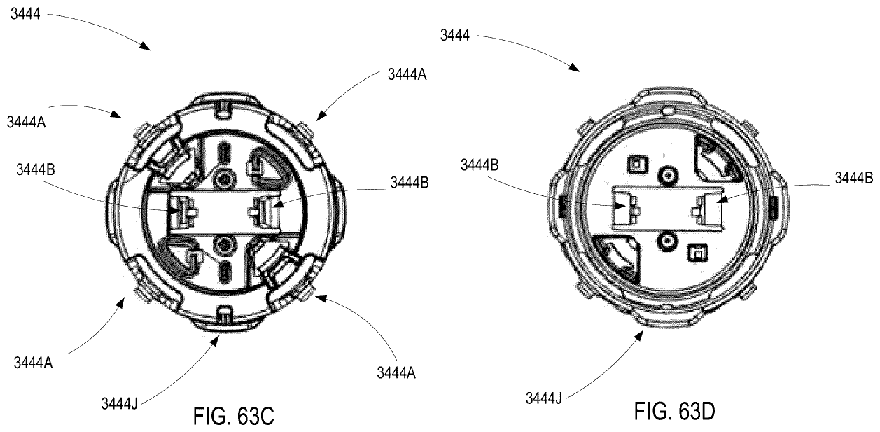

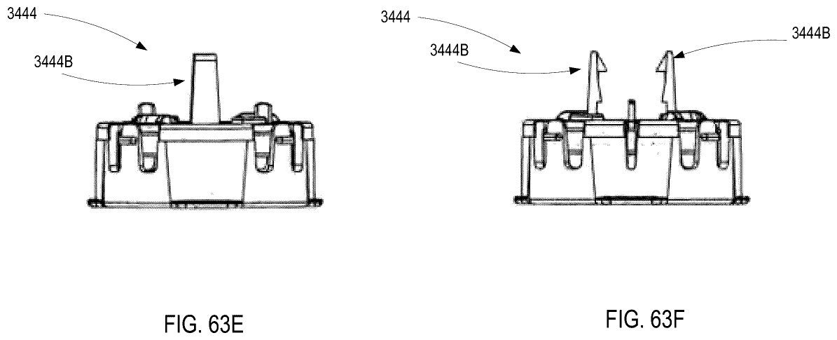

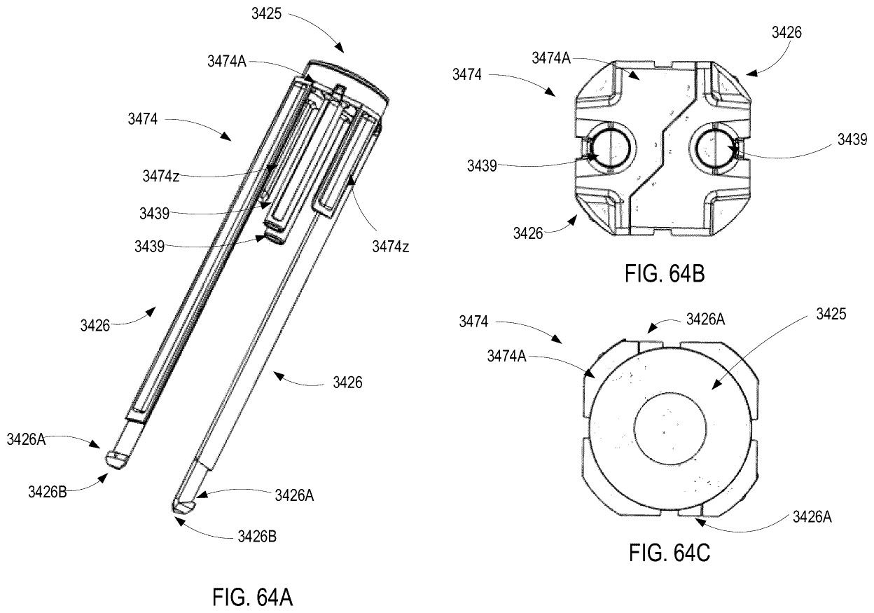

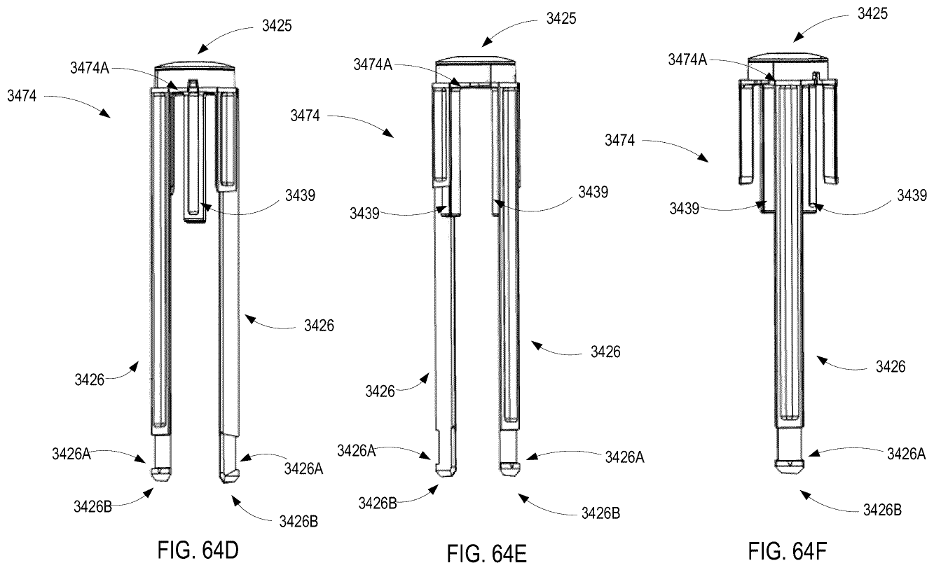

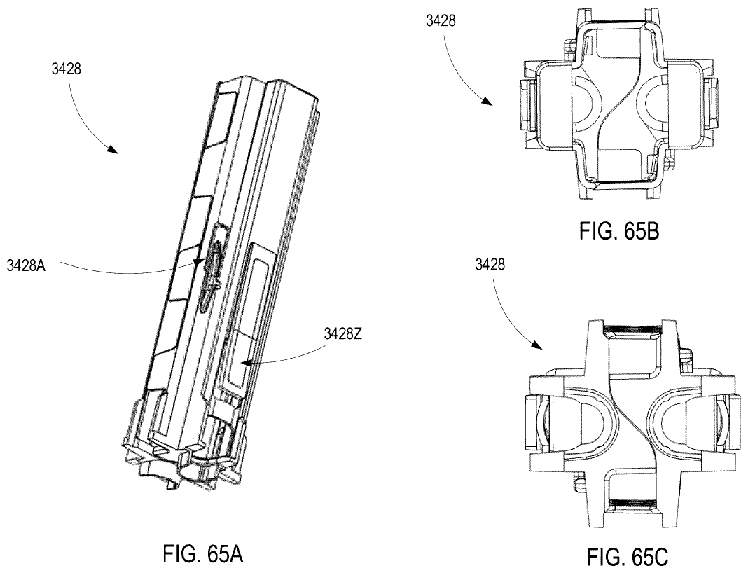

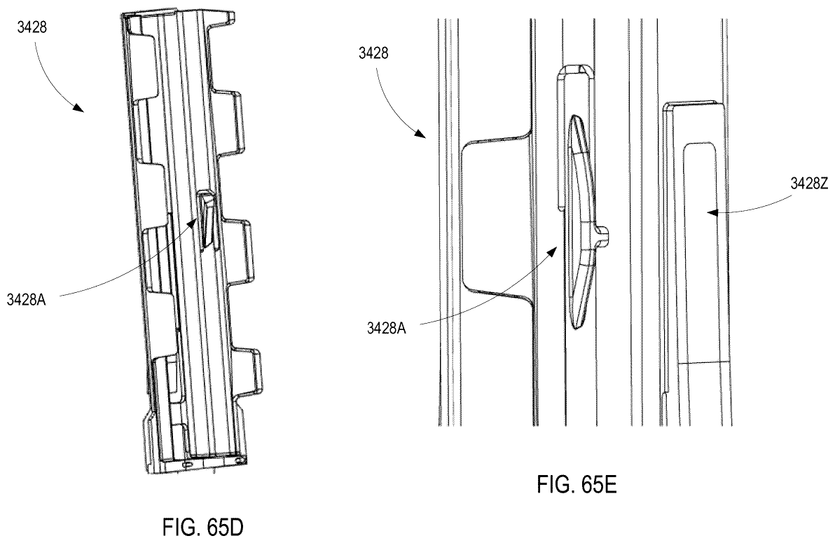

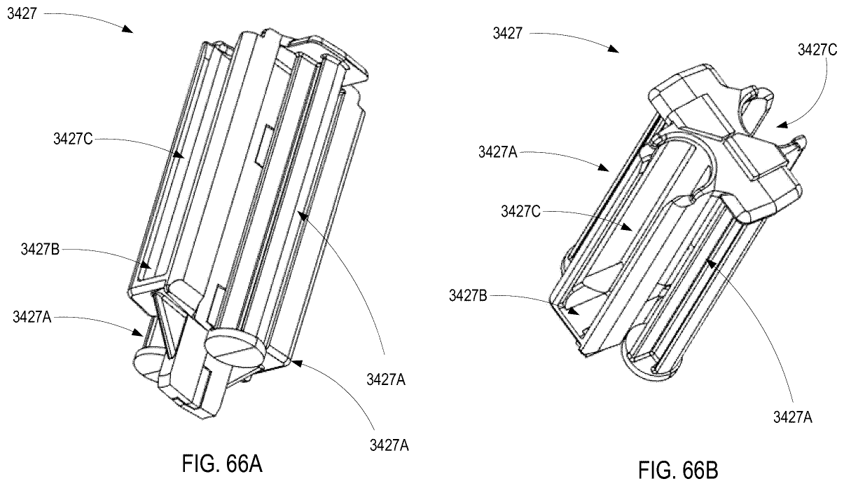

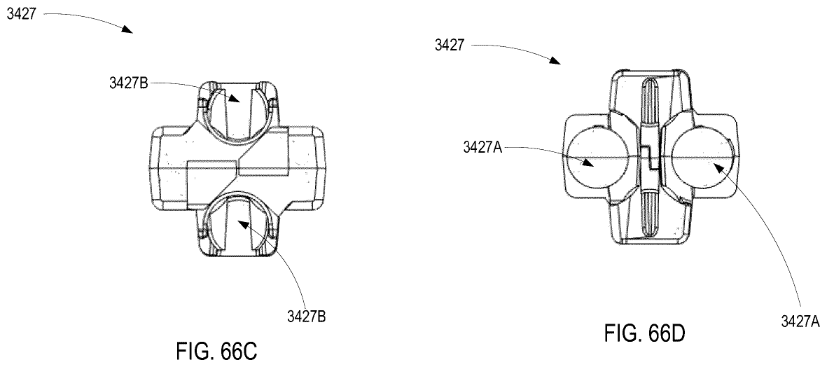

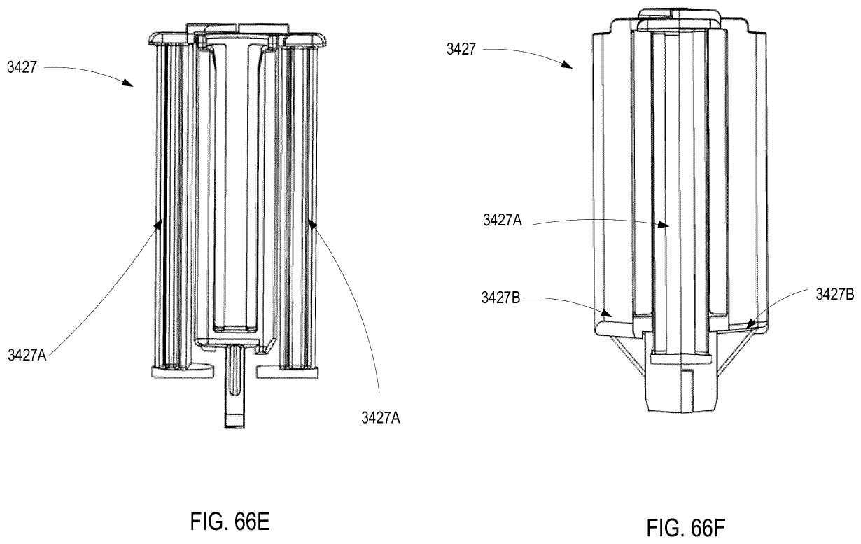

FIG. 56A-62D are various views of a bleeding treatment system 3400. FIGS. 56A-E are a perspective view, a front view, a side view, a bottom view, and a top view, respectively, of the bleeding treatment system 3400 in an initial, unactuated configuration. FIGS. 56F-56J are a perspective view, a front view, a side view, a bottom view, and a top view, respectively, of the bleeding treatment system 3400 in an activated configuration. FIGS. 57A and 57B are exploded views of the bleeding treatment system 3400. FIGS. 58A-58D are a front cross-sectional view, a right side cross-sectional view, a front 45 degree cross-sectional view, and a front 135 degree cross-sectional view of the system 3400 in the initial, unactuated configuration. FIGS. 59A-59D are a front cross-sectional view, a right side cross-sectional view, a front 45 degree cross-sectional view, and a front 135 degree cross-sectional view of the system 3400 in the actuated configuration. FIGS. 60A-60C are front 45 degree cross-sectional views of the system 3400 during various stages of ejecting an applicator pad 3410 and backing portion 3412 of the system 3400 from a grip 3440 of the system 3400. FIGS. 61A and 61B are schematic illustration of a perspective view of the system 3400 with a cap 3420 of the system 3400 in a locked and unlocked configuration, respectively. FIG. 61C is a schematic illustration of a side view of the system 3400 with the cap 3420 separated from the remainder of the system 3400.

The bleeding treatment system 3400 can be the same or similar in structure and/or function to any of the bleeding treatment systems disclosed herein. For example, the bleeding treatment system 3400 can include a grip 3440 including a first housing 3442 (also referred to as a first housing portion) and a second housing 3444 (also referred to as a second housing portion). The bleeding treatment system 3400 can also include an applicator pad 3410, a pair of reservoirs 3450, and an actuation mechanism 3498 (also referred to as an energy storage member or element).

As shown in FIGS. 57A and 57B, the bleeding treatment system 3400 also includes an optional removable distal cap 3420, a backing portion 3412 (also referred to as a cup or sponge cup), a pair of elongated members 3429 coupled to the second housing portion 3444 and extending proximally therefrom, and a pair of piercing members 3499 fixedly coupled to the second housing portion 3444. In some implementations, the second housing portion 3444, the piercing members 3499, and the pair of elongated members 3429 can be included in a lower actuator subassembly 3404. The bleeding treatment system 3400 also includes an optional ejection subassembly 3406 including an ejection button 3425, a pair of pad-urging elongated members 3426, and a pair of spring-receiving elongated members 3439 (also referred to as spring-supporting elongated members 3439). In some embodiments, the ejection button 3425 can be coupled to or included in an applicator pad release mechanism 3474 (also referred to as an ejector mechanism or an ejection mechanism) that includes the pair of pad-urging elongated members 3426 and the spring-receiving elongated members 3439 coupled to and extending distally from a base portion 3474A of the applicator pad release mechanism 3474. The bleeding treatment system 3400 also includes an inner housing subassembly 3405 including an inner frame 3428 (also referred to as an inner housing or an inner support frame) and a drive plunger 3427 including a pair of elongated plunger members 3427A configured to engage with respective reservoirs 3450 of the pair of reservoirs 3450 to urge fluid from the reservoirs 3450 upon activation of the activation mechanism 3498.

The applicator pad 3410 can be the same or similar in structure and/or function to any of the applicator pads described herein. In some embodiments, the applicator pad can have a thickness of between about ⅛ inch and about 1 inch, between about, ¼ inch and about 6/8 inch, between about ¼ inch and about ½ inch, about ⅝ inch, and/or about ¼ inch. In some embodiments, the applicator pad 3410 can have any suitable diameter, such as about 42 mm. In some embodiments, the applicator pad 3410 can be configured to have any suitable fluid capacity in an uncompressed configuration of the applicator pad 3410 (e.g., about 36 mL, about 30 mL, about 40 mL, between about 30 and 40 mL, between about 20 and 50 mL, between about 20 and 20 mL, and/or between about 10 and 30 mL). In some embodiments, the applicator pad 3410 can be configured to be about 80%, about 70%, about 60%, about 90%, about 70-90%, and/or any other suitable percentage saturated by the fluid when compressed (e.g., against a surface or wound of a user) and the fluid is fully dispensed to the applicator pad 3410. In some embodiments, when compressed, the applicator pad 3410 can be configured to have a fluid capacity of, for example, 3 mL, 4 mL, 5 mL, 6 mL, 7 mL, 8 mL, 9 mL, 10 mL, and/or any suitable range such as a range bounded by or containing these example volumes. For example, in some embodiments, the applicator pad 3410 can have a 7 mL capacity for 100% saturation and/or a 6 mL capacity for an 85% compressed saturation or 17% uncompressed saturation. The applicator pad 3410 can be configured to absorb blood from the wound while receiving, containing and/or dispensing the fluid from the reservoir 3450 to the wound.

The first housing portion 3442 can define an interior space and an open distal end. As shown, in some implementations the first housing portion 3442 can have a central axis, an elongated portion 3442X, and a base portion 3442W disposed distally of the elongated portion 3442X and having a greater lateral extent relative to the central axis than the elongated portion 3442X. The base portion 3442W can define the open distal end, the elongated portion 3442X can define a first portion of the interior space, and the base portion 3442W can define a second portion of the interior space. The first housing portion 3442 can also define an open proximal end or an opening in the proximal end within which the ejection button 3425 can be disposed. The first housing portion 3442 (e.g., the elongated portion 3442X) can also define at least one sidewall opening 3441A (e.g., two sidewall openings) such that at least one of the reservoirs 3450 can be viewed by a user through the sidewall openings 3441A. The first housing portion 3442 can include a transparent window segment disposed within each of the sidewall openings 3441A. In some implementations, the sidewall openings 3441A can be sufficiently long such that a majority of or the entire length of each reservoir 3450 and/or of a travel path of a piston of each reservoir 3450 can be viewed through the sidewall openings 3441A. The first housing portion 3442 can optionally include a gripping texture, such as ribs, disposed on the outer surface of the first housing portion 3442 (e.g., an outer surface of the elongated portion 3442X) to improve the grip of the user.

In some embodiments, each of the components of the system 3400 disposed within the interior space of the first housing portion 3442 can optionally include stabilizing portions (e.g., elongated members) for guiding, stabilizing, and/or supporting movement of the component within the interior space of the first housing portion 3442 (e.g., within voids formed by other components of the system 3400). For example, the applicator pad release mechanism 3474 can include stabilizing portions 3474z. Additionally, in some embodiments, the base portion 3442W, a proximal surface of the base portion 3442W, a portion of the second housing portion 3444 (e.g., a planar surface or portion), a distal surface of the inner housing 3428, and/or a proximal or substantially planar surface of the backing portion 3412 can function separately or collectively as a force distribution plate as described with respect to other embodiments herein.

The second housing portion 3444 can be configured to be at least partially disposed in the interior space of the first housing portion 3442 (e.g., at least partially disposed in the interior space of the base portion 3442W). FIGS. 63A-F are a top perspective view, a bottom perspective view, a top view, a bottom view, a front view, and a side view, respectively, of the second housing portion 3444. The second housing portion 3444 can have a distal end disposed distally of the distal end of the first housing portion (e.g., of the base portion 3442W) in an initial configuration of the second housing portion 3444 relative to the first housing portion (e.g., in the initial, unactuated configuration of the system 3400 shown in FIGS. 56A-56E). In the actuated configuration of the system 3400, the distal end of the second housing portion 3444 can be disposed distally of the distal end of the first housing portion or coplanar with the distal end of the first housing portion. The second housing portion 3444 can include a proximal wall and sidewalls having a perimeter having a similar shape to the perimeter of the applicator pad 3410 and/or the backing portion 3412 (e.g., a substantially circular shape). Thus, the second housing portion 3444 can define an interior space configured to receive at least a portion of the applicator pad 3410 and/or the backing portion 3412. The portion of the interior space defined by the base portion 3442W of the first housing portion 3442 can have a substantially complementary shape to an outer surface of the second housing portion 3444. The applicator pad 3410 can be disposed distally of (e.g., coupled to) a distal-facing surface of the second housing portion 3444 in the initial, unactuated configuration of the system 3400 and can extend distally of a distalmost surface of the second housing portion 3444. In some embodiments, the applicator pad 3410 can also be disposed distally of a distalmost surface of the second housing portion 3444 in the actuated configuration of the system 3400 (e.g., when compressed against a surface of a subject and/or after being removed from the surface of the subject).

As shown in FIG. 58B, the second portion 3444 can include proximally-extending retention elements 3444B (e.g., latches) configured to engaged with a portion of the inner housing 3427 (e.g., a distal surface) to retain the second housing portion 3444 relative to the inner housing 3427 and the first housing portion 3442 so that the second housing portion 3444 does not inadvertently separate from the first housing portion 3442 (e.g., prior to use of the system 3400). The second housing portion 3444 can include one or more latch elements 3444A configured to be received within respective latch openings 3442Y defined by the first housing portion 3442 (e.g., the base portion 3442W) such that, upon the first housing portion 3442 being translated fully distally relative to the second housing portion 3444 (e.g., from a first position to a second position sufficiently distal of the first position such that the system 3400 is actuated and fluid is dispensed to the actuator pad 3410), the latch elements 3444A can be received within the latch openings 3442Y such that the second housing portion 3444 is retained in a position more proximal than the initial position of the second housing portion 3444 relative to the first housing portion 3442. For example, the latch elements 3444A can be biased outward but can be urged inward by the inner surface of the base portion 3442W when the system 3400 is in the initial, unactuated configuration and as the first housing portion 3442 is moved distally relative to the second housing portion 3444. Upon the first housing portion 3442 reaching its fully distal position relative to the second housing portion 3444, the latch elements 3444A can align with the latch openings 3442Y such that the latch elements 3444A are no longer retained in an inward configuration by the inner surface of the base portion 3442W, and the latch elements 3444A can expand outward into the latch openings 3442Y to prevent the second housing portion 3444 from translating distally relative to the first housing portion 3442. The second housing portion 3444 can have any suitable number of latch elements 3444A and the first housing portion 34442 can have any suitable number of complementing latch openings 3442Y, such as, for example, one, two, three, four, five, or more.

The backing portion 3412 can include a proximal wall and sidewalls collectively defining an interior space configured to receive at least a portion of the applicator pad 3410. As shown in FIGS. 62A-62D, which are a proximal view, a distal view, a side view, and a cross-sectional view of the backing portion 3412 and the applicator pad 3410, the applicator pad 3410 can be disposed partially in the interior space of the backing portion 3412 and can extend distally of the backing portion 3412 in an uncompressed and/or a compressed state (e.g., when pressed against a surface of a subject). The backing portion 3412 can define openings 3412A through the proximal wall through which fluid can flow from the reservoirs 3450 to the applicator pad 3410 (e.g., an opening 3412A per reservoir 3450). Additionally, the backing portion 3412 can include retention elements 3412C (e.g., a pair of oppositely-disposed retention elements 3412C) configured to mate with an inwardly-facing retention flange, rim, recess, or groove 3444G disposed on an inner surface of a sidewall of the second housing portion 3444 to prevent the backing portion 3412 from separating from the second housing portion 3444 inadvertently prior to an optional ejection step. The backing portion 3412 can also include a set of outwardly-extending projections 3412E configured to mate with a recess or groove 3444H defined in an inwardly-facing surface of the second housing portion 3444 such that when a proximal force is applied to the backing portion 3412 (e.g., by the applicator pad 3410 due to pressing the applicator pad 3410 against a surface of a subject while urging the first housing portion 3442 distally toward the surface), the backing portion 3412 urges the second housing portion 3444 proximally into the interior of the first housing portion 3442. As shown in FIG. 62D, the backing portion 3412 can include circumferential sealing portions 3412F disposed about each opening 3412A and configured to form a fluid seal when engaged with a distal extending portion of the second portion 3444 defining an opening 3499K within which a piercing member 3499 is disposed, as shown in FIG. 59A. Thus, fluid that is dispensed through the piercing members 3499 and through the openings 3412A can form a fluid bolus disposed between the distal-facing surface 3412B of the backing portion 3412 and a proximal surface of the applicator pad 3410 and/or within the openings 3412A as the fluid is absorbed into the applicator pad 3410.

The backing portion 3412 can be coupled to the applicator pad 3410 via any suitable coupling mechanism. For example, the applicator pad 3410 can be coupled to the backing portion 3412 via a friction fit and/or adhesive. In some embodiments, adhesive can be disposed between the applicator pad 3410 and an inner surface of the sidewall of the backing portion 3412. In some embodiments, alternatively or in addition, adhesive can be disposed between the proximal surface of the applicator pad 3410 and the distal surface of the backing portion 3412.

In some embodiments, as shown in FIG. 62B, the distal surface of the proximal wall of the backing portion 3412 can be smooth. In some embodiments, the distal surface of the proximal wall or the backing portion 3412 can include fluid flow channels as shown and described with respect to other embodiments. In some embodiments, as shown, the distal surface of the proximal wall of the backing portion 3412 can be convex. In some embodiments, the distal surface of the proximal wall of the backing portion 3412 can be flat or concave.

The backing portion 3412 can be formed of any suitable material, and can be flexible or rigid. For example, the backing portion 3412 can include webbing features for improved structural integrity (e.g., disposed around a perimeter of a proximal surface of the backing portion 3412), and can optionally have fewer or no webbing features in a central area of the backing portion 3412 to improve flexibility of the backing portion 3412. In some embodiments, the backing portion 3412 can be formed of thermoplastic polyurethane (TPU) and/or polypropylene (PP). In some embodiments, some portions of the backing portion 3412 can be displaced relative to other portions via a user pressing on the proximal side of the backing portion 3412 such that a user can control compression of the backing portion 3412 and the applicator pad 3410 (e.g., compression of a particular portion of the applicator pad being greater than another portion of the applicator pad) relative to the wound when the backing portion 3412 and the applicator pad 3410 are decoupled from the remainder of the system 3400.

The reservoirs 3450 can each be formed as a cartridge including a rigid housing (e.g., a barrel) defining an interior configured to be prefilled with a medication (e.g., liquid). Each reservoir 3450 can have a distal open end closed by the release mechanism 3460 (e.g., a pierceable membrane formed as a cap or cover) and a proximal open end closed by a movable piston 3451. The release mechanism 3460 can be the same or similar in structure and/or function to any of the release mechanisms described herein (e.g., the release mechanism 2360). In the initial configuration of the system 3400, the movable piston 3451 is located at a proximal end of the housing of the reservoir 3450, as shown in FIG. 58A, and disposed at least partially within housing of the reservoir 3450. The movable piston 3451 is configured to be translated from the proximal end of the reservoir 3450 to a distal end of the reservoir 3450 to urge the fluid from the reservoir 3450 (e.g., when the release mechanism 3460 is pierced by the piercing member 3499 to allow flow from the reservoir 3450).

Each piercing member 3499 defines a lumen through which liquid can flow from the reservoir toward the applicator pad 3410. As shown in FIG. 58A, each piercing member 3499 is fixedly coupled to the second housing portion 3444 and aligned with, but not in piercing contact with, a release mechanism 3460 of a reservoir 3450. Each piercing member 3499 can have a sharp proximal end and a blunt distal end. Each piercing member 3499 can be coupled to the second housing portion 3444 such that the each piercing member 3499 has a distal end that is proximal of a distal portion of the second housing portion 3444 defining an opening within which each piercing member 3499 is disposed. Thus, each piercing member 3499 is disposed and sized to not contact the applicator pad 3410 or the backing portion 3412 during operation of the system 3400.

FIGS. 65A-65C are a perspective view, a top view, a bottom view, respectively, of the inner support frame 3428. FIG. 65D is a perspective view of an inner side of an elongated half of the inner support frame 3428. FIG. 65E is an enlarged view of a portion of an outer surface of the inner support frame 3428. The inner support frame 3428 can be disposed within the interior space defined by the first housing portion 3442 and can be fixedly coupled to the first housing portion 3442 such that the inner support frame 3428 and the first housing portion 3442 translate together. The inner support frame includes a pair of outwardly extending tabs 3428Z that are configured to be received in recesses defined in the first housing portion 3442 such that the inner support frame 3428 is coupled to the first housing portion 3442. The inner support frame 3428 defines parallel channels within which each of the two reservoirs 3450 are disposed and retained such that the inner support frame 3428 and the reservoirs 3450 translate together.

FIGS. 66A-66F are a lower perspective view, an upper perspective view, a top view, a bottom view, a front view, and a side view, respectively, of the drive plunger 3427. The drive plunger 3427 is disposed within the interior space defined by the first housing portion 3442 and includes a pair of plunger members 3427A configured to translate within the channels of the inner support frame 3428 to urge the pistons 3451 distally relative to the reservoir housings of the reservoirs 3450. Prior to actuation, the drive plunger 3427 is retained in a non-urging position relative to the reservoirs 3450 and the actuation mechanism 3498 is retained in an energy storage configuration by the drive plunger 3427. For example, each spring 3498 of the actuation mechanism 3498 can be biased toward an expanded configuration and can be disposed in a compressed configuration between a surface 3427B of the drive plunger 3427 and a proximal surface of the system 3400 (e.g., a surface of the first housing portion 3442 and/or a surface of the ejection subassembly) in an initial, unactuated configuration of the system 3400. Each spring 3498 can be at least partially disposed in a spring channel 3427C defined in the drive plunger 3427. The drive plunger 3427 is retained in the initial configuration by a pair of retention features 3428A of the inner support frame 3428 that are biased toward an outward position but are retained in an inward position engaging a distal surface of the drive plunger 3427 and preventing distal movement of the drive plunger 3427 due to the elongated members 3429 coupled to the second housing portion 3444 being disposed between the retention features 3428A of the inner support frame 3428 and the inner surface of the first housing portion 3442 to prevent outward movement of the pair of retention features 3428A (e.g., relative to a central axis of the first housing portion 3442). Each of the elongated members 3429 defines an inwardly-facing recess 3429A. When the first housing portion 3442 is translated distally relative to the second housing portion 3444 to the actuated position, the recesses of the elongated members 3429 align with the retention features 3428A, allowing the retention features 3428A to expand outward from the retaining position to a non-retaining position such that the drive plunger 3427 can be driven distally by the expanding springs and the plunger members 3427A can urge the pistons 3451 distally within the reservoir housings of the reservoirs 3450.

Due to the piercing members 3499 being fixedly coupled to the second housing portion 3444 and the reservoirs 3450 being retained in a fixed position relative to the first housing portion 3442, translation of the first housing portion 3442 distally relative to the second housing portion 3444 can cause the piercing members 3499 to piercingly contact the release mechanisms 3460 of the reservoir 3450 to allow fluid to flow from the reservoirs 3450 to the applicator pad 3410 via the piercing members 3499 and the openings in the backing portion 3412.

FIGS. 64A-C are a perspective view, a bottom view, and a top view, respectively of the ejection subassembly 3406. FIGS. 64D-F are side views from various angles of the ejection subassembly 3406. As shown, the ejection subassembly 3406 can include a base portion 3474A from which the elongated members 3426 and the spring-receiving elongated members 3439 extend distally (e.g., forming the applicator pad release mechanism 3474). The springs 3498 can be configured to receive the spring-receiving elongated members 3429 within an interior lumen of the springs 3498 such that the proximal end of each of the springs 3498 is contacting or mounted to the base portion 3474A of the ejection subassembly 3406 and each of the springs 3498 are coiled around the elongated member 3429 in an initial configuration of the system 3400.

The pair of pad-urging elongated members 3426 can be coupled to the ejection button 3425 (e.g., via the base portion 3474A of the ejection subassembly 3406) and extend distally thereof. Each elongated member 3426 can include a retention element 3426A (e.g., a hook or latch) configured to selectively mate with a complementary retention element 3444K of the second housing portion 3444. In some embodiments, as shown, the retention element 3426A can be formed of a reduced thickness portion of the elongated member 3426 and a hook or latch element disposed on the distal end of the reduced thickness portion of the elongated member 3426 such that a recess is formed in the elongated member 3426. As shown in FIG. 58C, in the initial, non-actuated configuration of the system 3400, the retention element 3426A of each elongated member 3426 can be mated with the retention element 3444K of the second housing portion 3444 such that the ejection button 3425 is retained in a depressed position (e.g., an initial, unactivated position) relative to the proximal end of the first housing portion 3442 (e.g., is substantially co-planar with the proximal end of the first housing portion 3442 and/or no sidewall of the ejection button 3425 is visible). Thus, a user of the system 3400 is prevented from mistaking the ejection button 3425 for an actuation button.

The distal end 3426B of each elongated member 3426 (e.g., the distal end 3426B of the retention element 3426A) can be configured to mate with an ejection feature 3412F of the backing portion 3412 (e.g., a hub surrounding recess or opening and/or a raised or non-raised portion of the proximal surface 3412D of the backing portion 3412). Thus, as shown in FIG. 60A, as the first housing portion 3442 is translated distally relative to the second housing portion 3444, the retention elements 3426A of the elongated members 3426 will advance distally out of contact with the retention elements 3444K of the second housing portion 3444 and into engagement with the ejection feature 3412F of the backing portion 3412. Additionally, as shown in FIG. 60A, the expansion of the springs 3498 can urge the ejection button 3425 and proximal ends of the elongated members 3426 proximally (e.g., via the base portion 3474A of the ejection subassembly 3406) such that the ejection button 3425 translates to a position proximal of the initial position of the ejection button 3425 (e.g., an actuated configuration) and projects from the proximal end of the first housing portion 3442. In some embodiments, a sidewall of the ejection button 3425 and/or of a portion of the pad release mechanism 3474 can be viewed by the user in the actuated configuration of the ejection button 3425. The ejection button 3425 can then be pressed distally, as shown in FIG. 60B, to cause the elongated members 3426 to translate distally relative to the second housing portion 3444 (e.g., through openings in the second housing portion 3442) to urge the backing portion 3412 distally. The force applied by the elongated member 3426 on the backing portion 3412 due to the button 3425 being pressed distally can overcome the retention force between the backing portion 3412 and the second housing portion 3442 such that the backing portion 3412 and the applicator pad 3410 separate from the second housing portion 3444 and are uncoupled from the remainder of the system 3400. The applicator pad 3410 and the backing portion 3412 can then be used separately from the remainder of the system 3400 (e.g., by holding the applicator pad 3410 and backing portion 3412 against the subject with a hand or by maintaining the applicator pad 3410 and backing portion 3412 against the subject using a bandage or wrap).

As shown in FIGS. 61A-61C, the distal cap 3420 can optionally be included in the system 3400 and removed from the first housing portion 3442 prior to actuation of the system 3400. In some implementations, the cap 3420 can be formed of a transparent material. The cap 3420 can be configured to prevent movement of the first housing portion 3442 relative to the second housing portion 3444 prior to removal of the cap 3420. The cap 3420 can include a set of openings or slots 3421 configured to receive outwardly-projecting retention features 3444J of the second housing portion 3444 in the locked configuration of the cap 3420. The outwardly-projecting retention features 3444J can project from an outer surface (e.g., a bottom edge) of the second housing portion 3444 away from a central axis of the second housing portion 3444 and first housing portion 3442. Although four of each are shown, any suitable number of retention features 3444J and slots 3421 can be included, such as one, two, three, five, six, etc. As shown in FIG. 61A, when the cap 3420 is in the locked configuration, the retention features 3444J project through the slots 3421 and prevent the cap 3420 from being translated distally relative to the first housing portion 3444 and the second housing portion 3442 due to contact between the retention features 3444J and portions of the cap 3420 proximal of the slots 3421. To unlock the cap 3420, the cap can be rotated (e.g., 45 degrees) such that slots 3421 rotate out of alignment with the retention features 3444J and the retention features 3444J are disposed in an interior region of the cap rather than through the slots 3421. Thus, the retention features 3444J no longer interfere with a distal translation movement of the cap 3420. As shown in FIG. 61C, the cap can then be translated distally relative to the first housing portion 3442 and the second housing portion 3444 (e.g., in the direction of arrows A) to separate the cap 3420 from the second housing portion 3444 and expose the applicator pad 3410 for use on a subject. As shown, the cap 3420 can optionally include an arrow or other identifier on visible on the surface of the cap 3420 to signal to a user which direction the cap should be rotated to transition from the locked to the unlocked configuration. Optionally, the cap 3420 can include a blocking portion on one end of each slot 3421 to prevent rotation of the cap 3420 in a non-unlocking direction relative to the second housing portion 3444 by blocking further rotation of the retention features 3444J. In some embodiments, rather than including a cap 3420 as shown (e.g., a twist-off cap), the system 3400 can include any other suitable cap, such as a cap that is coupled to the first housing portion 3442 via mating threads, a force fit, and/or includes a hinge.

In use, a user (e.g., the subject or a caregiver) can use the system 3400 using the same or similar method steps as described with respect to any of the other systems and methods herein. For example, the user can remove the optional cap 3420 and place the absorbent pad 3410 in contact with a wound of the subject in the initial, unactuated state. The user can then translate the first housing portion 3442 distally toward the wound a sufficient distance to compress the absorbent pad 3410 against the wound and to cause the first housing portion 3442 to advance a sufficient distance (e.g., from a first position to a second position) relative to the second housing portion 3444 to cause the system 3400 to actuate. In some embodiments, the amount of actuation force applied to actuate the system 3400 (e.g., to translate the first housing portion 3442 from the first position to the second position) can be less than 2 N, between about 5 N and about 15 N, between about 2N and about 20 N, or any other suitable amount of force. The relative translation of the first housing portion 3442 and the second housing portion 3442 can cause the actuation mechanism 3498 to be actuated such that the springs apply pressure to the pistons of the reservoirs 3450 and (e.g., simultaneously or immediately before or after) can cause the piercing members 3499 to pierce the release mechanisms 3460 of the reservoirs 3450 such that a fluid coupling is established from the reservoirs 3450 to the applicator pad 3410 and fluid is urged through the fluid coupling by the drive plunger 3427 under control of the actuation mechanism 3498 (e.g., springs). In some embodiments, simultaneously or immediately before or after the actuation of the actuation mechanism 3498 and piercing of the release mechanisms 3460, the ejection button 3425 can also be urged proximally as a result of the relative movement of the first housing portion 3442 and the second housing portion 3444. The user can maintain the applicator pad 3410 against the wound for a period of time sufficient for the fluid to be dispensed to the applicator pad 3410 by continuing to grip the first housing portion 3442 (e.g., with fingers wrapped around the elongated portion 3442X) and pressing or pulling the first housing portion 3442 against the wound such that the applicator pad 3410 remains compressed. In some embodiments, the fluid can be dispensed to the applicator pad 3410 in a period of time of about 5 seconds, about 10 seconds, about 15 seconds, about 20 seconds, about 25 seconds, about 30 seconds, about 35 seconds, about 40 seconds, or within a range of time of about 3-10 seconds, of about 10-30 seconds, of about 20-30 seconds, of about 20-30 seconds, of about 5 seconds to about 30 seconds, of about 10 seconds to about 60 seconds, or any suitable amount or range of time. In some embodiments, the user can observe movement of the piston 3451 and/or plunger 3427A relative to the reservoir housing of the reservoir 3450 through the window 3441A of the first housing portion 3442 and evaluate whether the system 3400 is functioning properly and/or determine when the fluid has been fully dispensed.

Similarly as described with respect to other method steps described herein, the user can maintain the applicator pad 3410 against the wound for a period of time (e.g., a period of time sufficient to achieve hemostasis) by continuing to grip the first housing portion 3442 (e.g., with fingers wrapped around the elongated portion 3442X) and pressing or pulling the first housing portion 3442 against the wound such that the applicator pad 3410 remains compressed. The period of time can optionally be based on when the fluid was observed to be fully dispensed through the window 3441A. After a period of time, the user can check the wound and/or remove the applicator pad 3410 from the wound to discontinue use of the grip 3440. Optionally, the user can eject the applicator pad 3410 and backing portion 3412 from the remainder of the system 3400 (e.g., from the grip 3440) by pressing the ejection button 3425 distally (e.g., toward the second housing portion), causing the elongated members 3426 to apply a force to the backing portion 3412 to urge the backing portion 3412 and the applicator pad 3410 to separate from the second housing portion 3444 and be released from the grip 3440. Optionally, the applicator pad 3410 and the backing portion 3412 can then be applied to the wound for a period of time (e.g., by holding the applicator pad 3410 and backing portion 3412 against the subject with a hand or by placing the applicator pad 3410 against the wound and applying a bandage or wrap to the backing portion 3412 to secure the applicator pad 3410 and the backing portion 3412 relative to the wound).

While various embodiments have been described herein, textually and/or graphically, it should be understood that they have been presented by way of example only, and not limitation. Likewise, it should be understood that the specific terminology used herein is for the purpose of describing particular embodiments and/or features or components thereof and is not intended to be limiting. Various modifications, changes, enhancements, and/or variations in form and/or detail may be made without departing from the scope of the disclosure and/or without altering the function and/or advantages thereof unless expressly stated otherwise. Functionally equivalent embodiments, implementations, and/or methods, in addition to those enumerated herein, will be apparent to those skilled in the art from the foregoing descriptions and are intended to fall within the scope of the disclosure.

Where schematics, embodiments, and/or implementations described above indicate certain components arranged and/or configured in certain orientations or positions, the arrangement of components may be modified, adjusted, optimized, etc. The specific size and/or specific shape of the various components can be different from the embodiments shown and/or can be otherwise modified, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise. By way of example, in some implementations, a treatment device intended to provide treatment to an adult user may have a first size and/or shape, while a treatment device intended to provide treatment to a pediatric user may have a second size and/or shape smaller than the first size and/or shape. Moreover, the smaller size and/or shape of, for example, a pediatric treatment device may result in certain components being moved, reoriented, and/or rearranged while maintaining the desired function of the device.

Although various embodiments have been described as having particular characteristics, functions, components, elements, and/or features, other embodiments are possible having any combination and/or sub-combination of the characteristics, functions, components, elements, and/or features from any of the embodiments described herein, except mutually exclusive combinations or when clearly stated otherwise. Moreover, unless otherwise clearly indicated herein, any particular combination of components, functions, features, elements, etc. can be separated and/or segregated into independent components, functions, features, elements, etc. or can integrated into a single or unitary component, function, feature, element, etc.

Where methods described above indicate certain events occurring in certain order, the ordering of certain events may be modified. Additionally, certain of the events may be performed concurrently in a parallel process when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

1. A system, comprising:
a first housing portion defining an interior space and an open distal end;
a second housing portion configured to be at least partially disposed in the interior space, the second housing portion having a distal end disposed distally of the distal end of the first housing portion in an initial configuration of the second housing portion relative to the first housing portion;
an applicator pad coupled to a distal-facing surface of the second housing portion;
a reservoir configured to contain a fluid; and
an energy storage member configured to be actuated to urge the fluid from the reservoir to the applicator pad,
the first housing portion configured to be translated distally relative to the second housing portion to cause actuation of the energy storage member.

2. The system of claim 1, wherein the second housing portion is configured to cause actuation of the energy storage member in response to the applicator pad being placed in contact with a wound of a user and the first housing portion being translated distally toward the wound and relative to the applicator pad and the second housing portion.

3. The system of claim 1, wherein the applicator pad is disposed distally of the distal end of the first housing portion in the initial configuration of the second housing portion relative to the first housing portion.

4. The system of claim 1, wherein at least a portion of the applicator pad is disposed distally of the distal end of the second housing portion in the initial configuration of the second housing portion relative to the first housing portion.

5. The system of claim 1, wherein the reservoir is disposed in the interior space of the first housing portion.

6. The system of claim 1, wherein the reservoir includes a cartridge including a barrel having an open distal end and an open proximal end, a piston translatable within the barrel, and a pierceable membrane coupled to the distal end of the barrel.

7. The system of claim 1, wherein the energy storage member is disposed in the interior space of the first housing portion.

8. The system of claim 1, wherein the energy storage member includes a spring member and the actuation of the energy storage member includes releasing the spring member such that the spring member transitions from a stored energy configuration to an activated configuration.

9. The system of claim 1, further comprising a drive plunger, the energy storage member configured to urge the drive plunger against the reservoir to cause the fluid within the reservoir to be dispensed to the applicator pad.

10. The system of claim 9, wherein:
the at least one reservoir includes a cartridge including a barrel having an open distal end and an open proximal end, a piston translatable within the barrel, and a pierceable membrane coupled to the distal end of the barrel, and
the energy storage member includes a spring member contacting a proximal-facing surface of the drive plunger, the spring member configured to translate the drive plunger distally such that the drive plunger urges the piston distally within the barrel of the cartridge.

11. The system of claim 1, wherein the applicator pad includes a backing portion and a pad portion coupled to a distal-facing surface of the backing portion.

12. The system of claim 11, wherein the backing portion defines an interior space within which a portion of the pad portion is disposed in the initial configuration of the second housing portion relative to the first housing portion.

13. The system of claim 11, wherein the pad portion is fixedly coupled to the backing portion.

14. The system of claim 11, wherein the backing portion defines at least one opening through which the fluid can be transferred to the pad portion.

15. The system of claim 11, wherein the backing portion is releasably coupled to the second housing portion such that the applicator pad can be decoupled from the second housing portion.

16. The system of claim 15, further comprising an ejection mechanism releasably couplable to the backing portion and configured to urge the applicator pad distally to decouple the applicator pad from the second housing portion.

17. The system of claim 16, wherein the ejection mechanism includes an elongated member translatable through an opening defined by the second housing portion to urge the applicator pad distally relative to the second housing portion to decouple the applicator pad from the second housing portion.

18. The system of claim 17, wherein the ejection mechanism includes an ejection button, the ejection button disposed in an unactuated first position when the second housing portion is in the initial configuration relative to the first housing portion, the ejection button configured to transition from the first position to an actuated second position in response to the first housing portion being translated distally relative to the second housing portion to cause actuation of the energy storage member, the ejection button configured to be depressed in the actuated second position to translate the elongated member of the ejection mechanism to urge the applicator pad distally relative to the second housing portion.

19. The system of claim 18, wherein the ejection button is configured to be transitioned from the first position to the second position by a force applied on the ejection mechanism by a proximal end of the energy storage member.

20. The system of claim 1, wherein the translation of the first housing portion relative to the second housing portion causes translation of the reservoir into piercing engagement with a piercing member such that the fluid flows from the reservoir to the applicator pad.
